(19) United States
(12) Patent Application Publication
(10) Pub. No.: US 2024/0377404 A1
(43) Pub. Date: Nov. 14, 2024

(54) BIOLUMINESCENCE-TRIGGERED PHOTOCATALYTIC ACTIVATION

(71) Applicant: Promega Corporation, Madison, WI (US)

(72) Inventors: Rachel Friedman OHANA, Madison, WI (US); Thomas KIRKLAND, Madison, WI (US); Robin HURST, Madison, WI (US); Mark KLEIN, Madison, WI (US); Matt LARSEN, Madison, WI (US); Hui WANG, Madison, WI (US); Wenhui ZHOU, Madison, WI (US); Thomas MACHLEIDT, Madison, WI (US)

(21) Appl. No.: 18/613,432

(22) Filed: Mar. 22, 2024

Related U.S. Application Data
(60) Provisional application No. 63/492,061, filed on Mar. 24, 2023.

(51) Int. Cl. G01N 33/58; B01J 23/46; B01J 27/26; B01J 31/02; C09K 11/06; C12N 9/02; G01N 21/76

(52) U.S. Cl. CPC G01N 33/582; B01J 23/462; B01J 23/468; B01J 27/26; B01J 31/0208; C09K 11/06; C12N 9/0069; G01N 21/763; C12Y 113/12013

(57) ABSTRACT

Provided herein are systems, methods, and compositions for bioluminescence-triggered photocatalytic activation of molecular entities in a proximity-dependent manner, which can be actuated within biological systems. In particular, provided herein are bioluminescent proteins or complexes, luminophore substrates thereof, photocatalysts, and activatable molecular entities incorporating light-responsive moieties that restrict their activity; systems thereof; and methods for catalytically activating the activatable molecular entities via bioluminescence-triggered catalysis.

### CROSS-REFERENCE TO RELATED APPLICATION

The present invention claims priority to U.S. Provisional Patent Application Ser. No. 63/492,061, filed Mar. 24, 2023, which is hereby incorporated by reference in its entirety.

### SEQUENCE LISTING

The text of the computer readable sequence listing filed herewith, titled “PRMG_40198_202_SequenceListing.xml,” created Jul. 2, 2024, having a file size of 8,902, is hereby incorporated by reference in its entirety.

### FIELD

Provided herein are systems, methods, and compositions for bioluminescence-triggered photocatalytic activation of molecular entities in a proximity dependent manner, which can be actuated within biological systems. In particular, provided herein are bioluminescent proteins or complexes, luminophore substrates thereof, photocatalysts, and activatable molecular entities incorporating light-responsive moieties or conformations that restrict their activity; systems thereof; and methods for catalytically activating the activatable molecular entities via bioluminescence-triggered catalysis.

### BACKGROUND

The need to study dynamic microenvironments, signaling pathways, and molecular processes in physiologically relevant contexts created a demand for new functional biology tools enabling such analyses in live cells and complex models in a nondestructive fashion.

### SUMMARY

Provided herein are systems, methods, and compositions for bioluminescence-triggered photocatalytic activation of molecular entities in a proximity dependent manner, which can be actuated within biological systems. In particular, provided herein are bioluminescent proteins or complexes, luminophore substrates thereof, photocatalysts, and activatable molecular entities incorporating light-responsive moieties that restrict their activity; systems thereof; and methods for catalytically activating the activatable molecular entities via bioluminescence-triggered catalysis.

In some embodiments, provided herein is light-driven photocatalysis that leverages bioluminescence as the light source and utilizing it to activate activatable molecules (e.g., molecules incorporating light-sensitive moieties that restrict their activity; photoswitchable molecules capable of a light-driven conformational change from an inactive to active conformation, etc.). Such molecules include molecules with photolabile protecting groups (photocages), photo-switchable molecules (photoswitches), etc. In some embodiments, the activated molecules are available for subsequent interactions with target molecules (e.g., biomacromolecules), and/or detection. However, while the activatable molecules are activated by the systems and methods described herein, the activated molecules do not comprise highly reactive and/or short-lived functional groups (e.g., for labeling of biological molecules). Instead, exposure to light emitted from the luminophore/bioluminescent protein or complex either (1) facilitates the removal of a blocking moiety from a caged entity, thereby allowing the molecular entity to engage in its chemical/biological function, or (2) facilitates a conformational change in the molecular entity that results in repositioning of moieties within the molecular entity into an activated state (e.g., so the moieties can interact).

The components of the bioluminescence-driven photocatalytic systems herein include a bioluminescent light source (e.g., a luminophore and a luciferase or bioluminescent complex) and a pair comprising a light-sensitive catalyst (photocatalyst) and an activatable molecular entity (e.g., a caged molecule, a photo-switchable molecule, etc.). In some embodiments, upon exposure to a light stimulus from the bioluminescent source, the excited catalyst engages in activation of neighboring activatable molecules for subsequent interactions, detection, etc., within the surrounding environment. Catalyst activation through absorption of visible light offers temporal control over catalytic reactivity. The use of bioluminescence to trigger photocatalysis in a proximity-dependent manner (e.g., requiring localization of the bioluminescent light source and the photocatalyst) provides a mild and minimally destructive light source, reduced phototoxicity, and efficient light delivery for triggering catalysis in intact cell as well as spatial and temporal (+luminophore) control over catalyst activation, thereby increasing overall the spatiotemporal resolution of downstream molecular activation. In some embodiments, the bioluminescent light-source and the photocatalyst are bioconjugated to induce proximity between the light source and the catalyst.

One aspect of the present technology is the use of bioluminescence, i.e., light generated by the interaction of a luminophore with a bioluminescent protein or complex of peptide(s) and/or polypeptides, to activate a photocatalyst. Other aspects of the present technology include: the activation of an activatable molecule by a bioluminescence-activated photocatalyst, assembling components of a bioluminescence-driven system/method through the use of one or more conjugates of the components of the systems here (e.g., via protein fusions, capture agents/elements, linkers, etc.) that drive in-cell photocatalytic activation chemistries using spatiotemporally arranged components to increase specificity and decrease toxicity, etc.

In one exemplary embodiment, exposure of a bioluminescent protein to an appropriate luminophore generates light that triggers local photocatalytic activation of molecular entities (e.g., uncaging via bond cleavage and removal of a protecting group or conformational change of a photo-switchable molecule). The activated molecules are then available for interaction with biomacromolecules within their surrounding environment and/or detection. Such activated molecules can be leveraged for a broad range of spatiotemporally-controlled phenotypic, proteomic, and genomic analyses including detection, activation, inactivation, and degradation of proximal proteins and nucleic acids as well as probing and altering of biological processes.

In some embodiments, appropriate proximity between the bioluminescent protein and the photocatalyst is achieved by tethering the photocatalyst to the bioluminescent protein (directly or indirectly). In certain embodiments, the bioluminescent protein is made as a fusion with a capture agent (e.g., capture protein), and the photocatalyst is conjugated (e.g., via a linker) to a capture element. Binding of the capture agent to the capture element brings the bioluminescent protein and the photocatalyst into proximity to enable light produced by the bioluminescent protein and the luminophore to activate the photocatalyst.

In some embodiments, rather than using a bioluminescent protein, a multipart bioluminescent complex can be used as the light source for the photocatalytic system or method herein. The use of a bioluminescent complex that only generates light upon complementation of two or more components (e.g., peptide(s), and/or polypeptide(s)) offers several advantages for some systems and methods herein. For example, conjugating directly or indirectly (e.g., fusing, tethering, etc.) one or more components of the bioluminescent complex to other components of the system (e.g., photocatalyst, activatable molecule, target, etc.) ensures the proximity of that component to the bioluminescent complex upon light generation. Tethering of two components of the system to separate components of the bioluminescent complex ensures the proximity of those components upon light generation by the complex. If the photocatalyst is tethered to the first component of the bioluminescent complex (e.g., LgBiT or a circularly permuted LgBiT (See, e.g., U.S. patent application Ser. No. 17/105,925; incorporated by reference in its entirety)) that has high affinity for the second component of the bioluminescent complex (e.g., HiBiT), which is genetically fused to a target of interest then proximity between the photocatalyst and the second component of the bioluminescent complex is required for initiation of photocatalysis, thereby providing greater spatiotemporal control over the activation the photocatalyst and a modality agnostic approach for targeting the photocatalytic system to a site of interest (i.e., complementation and luminophore addition).

In some embodiments, the bioluminescent protein or a component of the multipart bioluminescent complex is inserted at an internal position within the capture agent. In some embodiments, a position within the capture agent is selected to increase efficiency of bioluminescence activation of the catalyst through greater proximity or favorable conformation.

In some embodiments, the bioluminescent protein or a component of the multipart bioluminescent complex is circularly permuted.

Because a bioluminescent protein, or the components of a bioluminescent complex (or fusions thereof with other components of the systems herein), can be expressed within a cell or delivered into a cell, such systems offer generation of light for initiating photocatalysis within a cell.

In some embodiments, a component of a system herein (e.g., a bioluminescent protein or a component of a bioluminescent complex) is fused to a protein/peptide that results in specific localization of the component within a cell. For example, the localization protein/peptide might localize in a cellular compartment, bind to a specific protein, bind to DNA or RNA, bind to a specific nucleic acid sequence, etc. By localizing the component within a cell or linking the component to a specific cellular component, the subsequent photocatalytic activation chemistries (e.g., uncaging via bond cleavage, removal a protecting group, or conformational change of a photoswitch) are similarly localized. In some embodiments, by localizing the system to a specific cellular target (e.g., protein, nucleic acid sequence, etc.), the activated molecule is capable of interacting with or acting upon a cellular target.

The systems and methods herein provide for bioluminescence-triggered catalytic activation of molecular entities (e.g., caged molecules, photo-switchable molecules, etc.) in a proximity dependent manner offering new functional biology tools to study dynamic environments and molecular process in physiologically relevant contexts, including live cells, complex cellular models, and model organisms. The technologies herein utilize a non-invasive, intrinsic light-source to activate light-sensitive catalysts, which can further engage in local activation of molecular entities that can interact with their environments, be detected, interact with neighboring molecules/biomacromolecules, etc. The systems herein can be leveraged for a broad range of spatiotemporally-controlled phenotypic, proteomic, and genomic analyses.

### BRIEF DESCRIPTION OF THE DRAWINGS

FIG. 1. A cartoon depiction of the bioluminescent triggered photocatalytic system utilizing a bioluminescent protein (NanoLuc) and a light sensitive photocatalyst, which upon absorption of light engages in activation of a caged or photo-switchable molecules that can subsequently engage in interaction with biomacromolecules within their surrounding environment.

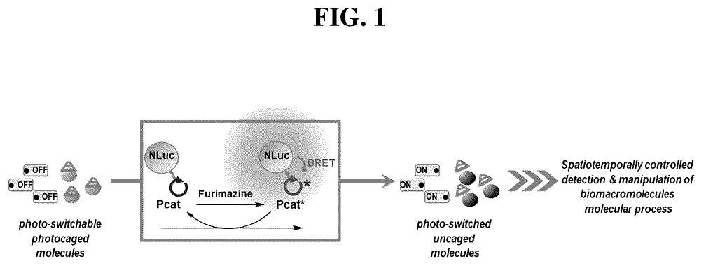

FIG. 2A-C. Exemplary structures of activatable molecules: FIG. 2A caged molecules that are uncaged via light-triggered photocatalytic cleavage of photolabile protecting groups, FIG. 2B caged molecule that is uncaged via light-triggered photocatalytic abstraction of a hydrogen (oxidation), FIG. 2C photo-switchable molecule that can undergo conformational change.

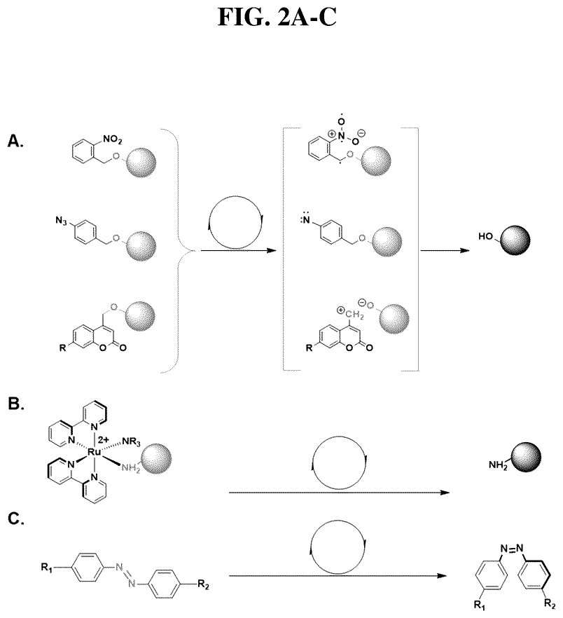

FIG. 3. A cartoon depiction of light sensitive catalyst that is modified to enable bioconjugation and subsequently proximity to the bioluminescent light source. Exemplary catalysts include iridium-based catalyst, ruthenium-based catalyst, and Rose Bengal (organic photosensitizer). R represents an attachment motif and Linker Q represents a bioconjugation motif. Exemplary bioconjugation motifs include 2-pyridinecarboxyaldehyde (PCA) and 2-cyanobenzothiazole (CBT) linkers for direct bioconjugation as well as chloroalkane for indirect conjugation via binding to a HaloTag fusion.

FIG. 4. Exemplary linker Qs designed for indirect conjugation via binding to a HaloTag fusion. The haloalkanes of varying lengths are designed for attachment to components of the systems herein (e.g., attachment to photocatalysts).

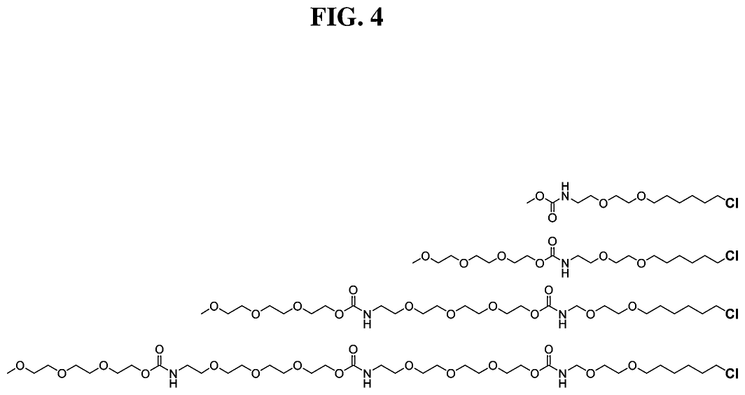

FIG. 5. The molecular structure of an exemplary photocatalysts linked to a HALOTAG substrate.

FIG. 6A-C. FIG. 6A A cartoon depiction of a system for localizing, inside cells, an extracellularly added or intracellularly-assembled haloalkane-linked photocatalyst with a bioluminescent complex component (LgBiT) genetically fused to a modified dehalogenase (HALOTAG). FIG. 6B Fluorescence experiments depicted subcellular localization of a LgBiT-HaloTag fusion, which is labeled with a fluorescent Haloalkane ligand. FIG. 6C Experiment demonstrating the binding kinetics of an extracellularly added haloalkane-linked Ir-photocatalyst to a LgBiT-HaloTag fusion localized to different subcellular compartment. Results shows complete binding within 60 minutes.

FIG. 7. A cartoon depiction of a system that allows for bioluminescence-triggered spatiotemporal molecular uncaging of caged effector molecules in intact cells. Complementation of HiBiT genetically fused to a protein of interest with LgBiT genetically fused to HaloTag and tethered to a catalyst allows localization of the catalyst, light source, and protein of interest enabling localized photocatalytic uncaging of caged effector molecules for subsequent manipulation of a site of interest.

FIG. 8. A cartoon depiction of a system for bioluminescence-triggered spatiotemporal turn-on of a photo-switchable molecule in intact cells. Complementation of HiBiT genetically fused to a protein of interest with LgBiT genetically fused to HaloTag and tethered to a catalyst allows localization of the catalyst, light source, and protein of interest enabling a localized reversible conformational switch and turn-on the activity of effector molecules toward a target of interest.

FIG. 9A-C. FIG. 9A Exemplary activatable molecules comprising an azide quenched fluorogenic dye. FIG. 9B Depiction of a system for bioluminescence-triggered spatiotemporal turn-on fluorescence. Complementation of HiBiT genetically fused to a protein of interest with LgBiT genetically fused to HaloTag and tethered to a catalyst allows localization of the catalyst, light source, and site of interest. FIG. 9C Depiction of a system for bioluminescence-triggered spatiotemporal fluorescence turn-on within the nucleus. Electroporation of nucleoprotein complex comprising a gRNA and a fusion of dCas9-NanoLuc-HaloTag tethered to the catalyst allows localization of a catalyst, light source, and site of interest.

FIG. 10A-B. FIG. 10A cartoon depiction of a system that allows for bioluminescence-triggered spatiotemporal molecular uncaging of caged fluorophore for subsequent detection of nucleic acids in intact cells. A caged fluorophore conjugated to an antisense oligo is targeted to a specific nucleic acid. The photocatalytic system is localized to a proximal nucleic acid sequence by either FIG. 10B a gRNA coupled with a fusion of dCas9-NanoLuc-HaloTag tethered to a catalyst or an antisense oligo conjugated to HiBiT. Complementation with LgBiT genetically fused to HaloTag and tethered to a catalyst allows localization of the catalyst, light source, and nucleic acid of interest. Upon treatment with furimazine the bioluminescent complex emits light, which triggers photocatalytic uncaging of the proximal fluorophore.

FIG. 11A-D. Influence of chloroalkane length on catalysts energy transfer efficiency, cell permeability and binding kinetic to HaloTag. FIG. 11A Structure of modifiable Ir-catalyst and its derivatives, which are further conjugated to a chloroalkane of different length. FIG. 11B Physiochemical properties of Ir-catalyst conjugates and their influence on energy transfer efficiency from NanoLuc to the Ir-catalyst. Influence of chloroalkane length on binding kinetic of chloroalkane-catalyst conjugates to HaloTag in either FIG. 11C cell lysate or FIG. 11D inside living cells.

FIG. 12A-D. Influence of chloroalkane length on catalysts energy transfer efficiency, cell permeability and binding kinetic to HaloTag. FIG. 12A Structure of modifiable Ru-catalyst and its derivatives, which are further conjugated to a chloroalkane of different length. FIG. 12B Physiochemical properties of Ru-catalyst conjugates and their influence on energy transfer efficiency from NanoLuc to the Ru-catalyst. Influence of chloroalkane length on binding kinetic of chloroalkane-catalyst conjugates to HaloTag in living cells within either the FIG. 12C cytosol or FIG. 12D the nucleus.

FIG. 13A-C. Optimization of the bioluminescent photocatalytic complex comprising bioluminescent energy donor, chloroalkane-catalyst conjugate and HaloTag offering the means to induce proximity between the two. FIG. 13A Scheme of the HT₁₇₈-cpNLuc-₁₇₉ chimera or the complementation-based HT₁₇₈-cpmLgBiT-₁₇₉ chimera comprising a circularly permuted NanoLuc (i.e., cpNLuc) or a circularly permuted mutant LgBiT (i.e., cpmLgBiT) inserted into a HaloTag's surface loop (between residues 178-179), which is proximal to the ligand interaction site. Brightness as well as BRET efficiency to a bound HaloTag TMR-fluorescent ligand for FIG. 13B NanoLuc-HaloTag fusion and chimera and FIG. 13C LgBiT-HaloTag fusion and chimera that were complemented with a HiBiT peptide.

FIG. 14A-C. Bioluminescence-triggered uncaging of an azido-quenched coumarin. FIG. 14A Structure of the amino caged coumarin PBI-8977. FIG. 14B Fluorescence imaging of Hela cells expressing the HT₁₇₈-cpNLuc-₁₇₉ chimera and treated with increasing concentration of PBI-8977 as well as fluorofurimazine in the presence and absence of Ru-8974. FIG. 14C image densitometry demonstrating catalyst dependent turn-on fluorescence.

FIG. 15A-C. Bioluminescence-triggered uncaging of an azido-quenched Ethidium Bromide (EMA). FIG. 15A Structure of EMA. FIG. 15B Fluorescence imaging of HeLa cells expressing the HT₁₇₈-cpNLuc-₁₇₉ chimera and treated with increasing concentration of EMA as well as fluorofurimazine in the presence and absence of either Ru-8974 or Ir-9049. FIG. 15C image densitometry demonstrating catalyst dependent turn-on fluorescence.

FIG. 16A-B. Bioluminescence-triggered release of a signaling molecule from a caging transition metal complex in a biochemical setting. FIG. 16A cartoon depicting bioluminescent-triggered release of serotonin from a [Ru²⁺(bpy)₂]² caging complex. FIG. 16B Fluorescence scan of [Ru(bpy)₂(PMe₃)(5HT)]²+ that is caged or was exposed to 45 minutes of bioluminescence.

FIG. 17A-C. Bioluminescence-triggered uncaging upon Ru-catalyzed p-azidobenzyl reduction. FIG. 17A cartoon depicting bioluminescent-triggered photocatalytic cleavage of p-azidobenzyl-luciferin upon Ru-catalyzed azide-reduction to release luciferin. FIG. 17B Firefly (FFLY) luminescence upon incubation with p-azidobenzyl-luciferin that was pre-incubated with purified HT₁₇₈-cpNLuc-₁₇₉-tethtered and untethered to Ru-8974 in the presence and absence of fluorofurimazine, FIG. 17C Fold increase in FFLY luminescence upon bioluminescence or LED triggered photocatalytic uncaging of p-azidobenzyl-luciferin.

FIG. 18A-C. Bioluminescence-triggered uncaging upon catalyst catalyzed excitation of O-nitrobenzyl and subsequent photolysis. FIG. 18A cartoon depicting bioluminescent-triggered photocatalytic cleavage of o-nitrobenzyl-F-luciferin to release F-luciferin. FIG. 18B FFLY luminescence upon incubation with o-nitrobenzyl-F-luciferin that was preincubated with purified HT₁₇₈-cpNLuc-₁₇₉-tethtered and untethered to Ru-8974 in the presence and absence of fluorofurimazine, FIG. 18C Fold increase in FFLY luminescence upon bioluminescence or LED triggered photocatalytic uncaging of o-nitrobenzyl-F-luciferin.

FIG. 19. Bioluminescence-triggered photocatalytic uncaging upon excitation of a Coumarin Derivative. Cartoon depicting bioluminescence-triggered photocatalytic photolysis upon excitation of Coumarin-4-methyl to release Ibrutinib from 6-bromo 7-hydroxy coumarin-4-methy-Ibrutinib.

FIG. 20. Bioluminescence-triggered isomerization of azobenzene. Cartoon depicting bioluminescence-triggered catalytic photoisomerization of azobenzene, which upon excitation possess a significantly lower energetic barrier for rotation around the N═N double bond allowing for conformational change that impacts either the distance and/or orientation of R1 and R2 with respect to each other. Such spatiotemporally controlled conformational change could be used to dramatically increase the affinity of effector molecules toward biomacromolecule targets.

### Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a peptide” is a reference to one or more peptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “system” refers a group of devices, reagents, compositions, etc. that are collectively grouped for a desired function or objective. The components of the system may reside in a single reaction mixture, cell, container, etc. or may be maintained separately, e.g., for subsequent combination to achieve the desired function or objective.

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-luminescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., luminescent intensity of a bioluminescent protein or bioluminescent complex).

As used herein, the term “luminescence” refers to the emission of light by a substance as a result of a chemical reaction (“chemiluminescence”) or an enzymatic reaction (“bioluminescence”).

As used herein, the term “bioluminescence” refers to production and emission of light by a reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light.

As used herein, the term “luminophore” refers to a chemical moiety or compound that can be placed in an excited electronic state (e.g., by a chemical or enzymatic reaction) and emits light as it returns to its electronic ground state.

As used herein, the term “imidazopyrazine luminophore” refers to a genus of luminophores including “native coelenterazine” as well as synthetic (e.g., derivative or variant) and natural analogs thereof, including furimazine, furimazine analogs (e.g., fluorofurimazine) coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); U.S. Pat. No. 8,669,103; U.S. Prov. App. No. 63/379,573; the disclosures of which are incorporated by reference herein in their entireties.

As used herein, the term “coelenterazine” refers to the naturally-occurring (“native”) imidazopyrazine of the structure:

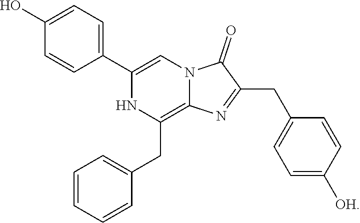 [SMILES: O=c1c(Cc2ccc(O)cc2)nc2c(Cc3ccccc3)[nH]c(-c3ccc(O)cc3)cn1-2]

As used herein, the term “furimazine” refers to the coelenterazine derivative of the structure:

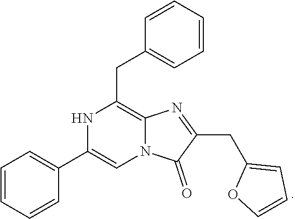 [SMILES: O=c1c(Cc2ccco2)nc2c(Cc3ccccc3)[nH]c(-c3ccccc3)cn1-2]

As used herein, the term “fluorofurimazine” refers to the furimazine derivative of the structure:

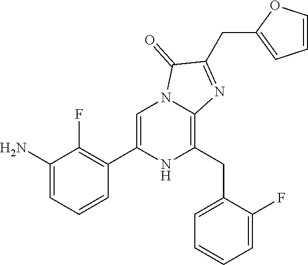 [SMILES: Nc1cccc(-c2cn3c(=O)c(Cc4ccco4)nc-3c(Cc3ccccc3F)[nH]2)c1F]

(U.S. application Ser. No. 16/548,214; incorporated by reference in its entirety).

As used herein, the term “luciferin” refers to a compound of the structure:

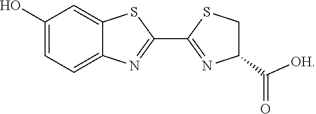 [SMILES: O=C(O)[C@H]1CSC(c2nc3ccc(O)cc3s2)=N1]

As used herein, the term “bioluminescence resonance energy transfer” (“BRET”) refers to the distance-dependent interaction in which energy is transferred from a donor bioluminescent protein/complex and substrate to an acceptor molecule without emission of a photon. The efficiency of BRET is dependent on the inverse sixth power of the intermolecular separation, making it useful over distances comparable with the dimensions of biological macromolecules (e.g., within 30-80 Å,depending on the degree of spectral overlap).

As used herein, the term “an Oplophorus luciferase” (“an OgLuc”) refers to a luminescent polypeptide having significant sequence identity, structural conservation, and/or the functional activity of the luciferase produced by and derived from the deep-sea shrimp Oplophorus gracilirostris. In particular, an OgLuc polypeptide refers to a luminescent polypeptide having significant sequence identity, structural conservation, and/or the functional activity of the mature 19 kDa subunit of the Oplophorus luciferase protein complex (e.g., without a signal sequence) such as SEQ ID NOs: 1 (NANOLUC), which comprises 10 β strands (β1, β2, β3, β4, β5, β6, β7, β8, β9, β10) and utilize substrates such as coelenterazine or a coelenterazine derivative or analog to produce luminescence.

As used herein the term “complementary” refers to the characteristic of two or more structural elements (e.g., peptide, polypeptide, nucleic acid, small molecule, etc.) being able to hybridize, dimerize, or otherwise form a complex with each other. For example, a “complementary peptide and polypeptide” are capable of coming together to form a complex. Complementary elements may require assistance (facilitation) to form a complex (e.g., from interaction elements), for example, to place the elements in the proper conformation for complementarity, to co-localize complementary elements, to lower interaction energy for complementary, to overcome low affinity for one another, etc.

As used herein, the term “complex” refers to an assemblage or aggregate of molecules (e.g., peptides, polypeptides, etc.) in direct and/or indirect contact with one another. In one aspect, “contact,” or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules (e.g., peptides and polypeptide) is formed under assay conditions such that the complex is thermodynamically favored (e.g., compared to a non-aggregated, or non-complexed, state of its component molecules). As used herein, the term “complex,” unless described as otherwise, refers to the assemblage of two or more molecules (e.g., peptides, polypeptides, or a combination thereof).

As used herein, the term “capture protein” or “capture agent” refers to a protein or other molecular entity that forms a stable covalent bond with its substrate, ligand, or other molecular clement upon interaction therewith. A capture protein may be a receptor that forms a covalent bond upon binding its ligand or an enzyme that forms a covalent bond with its substrate. An example of a suitable capture protein for use in embodiments of the present invention is the HALOTAG protein described in U.S. Pat. No. 7,425,436 (herein incorporated by reference in its entirety).

As used herein, the terms “capture ligand,” “capture moiety,” or “capture element” refers to a ligand, substrate, etc., that forms a covalent bond with a capture protein upon interaction therewith. An example of a suitable capture ligand for use in embodiments of the present invention is the HALOTAG ligand described, for example, in U.S. Pat. No. 7,425,436 (herein incorporated by reference in its entirety). Moieties that find use as HALOTAG ligands include haloalkane (HA) groups (e.g., chloroalkane (CA) groups). In embodiments described herein that specify an HA or CA capture ligand, other suitable capture ligands may be substituted unless otherwise specified.

As used herein, the term “activatable molecule” refers to a molecule capable of being converted from an activatable form (e.g., an inactive or inert form due to a blocking group of “cage” or an inactive conformation) to an activated (e.g., unblocked, uncaged, or active conformer) form by a catalyst. In some embodiments, the activatable molecule incorporates a light-responsive moiety that restricts its activity.

As used herein the term “caged molecule” refers to a molecule that has been rendered inactive (e.g., chemically inert, biologically inert, undetectable, etc.) by a chemical modification (e.g., blocking group) rendering them structurally or sterically blocked from functioning. Conversion of the caged molecule into an uncaged activated molecule comprises photocatalytic uncaging (e.g., cleavage of the blocking group) of the activatable molecule and liberation of the active from of the molecule (e.g., able to interact with a binding partner, detectable, etc.).

As used herein, the term “photoswitch” or “photoswitchable molecule” refers to a compound capable of adopting both active and inactive (or activatable) conformations. A conformational change, rather than a chemical one, results in photoswitching of the molecule from inactive to active. A reversible change in structural geometry, initiated by bioluminescence-triggered photocatalysis, activates the molecule.

As used herein, the term “cellular target” refers to any cellular (e.g., intracellular or surface exposed) entity (e.g., molecule, cellular compartment, complex, etc.) with which an activatable molecule (e.g., caged molecule, photoswitch) can interact. Cellular targets may be biomacromolecules such as protein, polypeptide, nucleic acid (e.g., DNA or RNA), lipids, polysaccharide, or a complex comprising any of these with a polypeptide(s). A cellular target could be composed of more than one component, subunit, or polypeptide, e.g., the cellular target is a protein complex. Examples of a cellular target may include a receptor or an enzyme.

As used herein, the term “bioactive agent” refers generally to any physiologically or pharmacologically active substance or a substance suitable for detection. In some embodiments, a bioactive agent is a potential therapeutic compound (e.g., small molecule, peptide, nucleic acid, etc.), or drug-like molecule. Bioactive agents for use in embodiments described herein are not limited by size or structure.

As used herein, the term “photocatalyst” refers to a molecule that, upon absorption of light at an appropriate wavelength, is capable of engaging in activation of a neighboring activatable molecules via either energy transfer or electron transfer events, thereby converting the activatable molecule into an activated states and/or lowering the activation energy and/or increasing the rate of a chemical reaction. In some embodiments, the excited photocatalyst is capable of regenerating itself after each energy transfer or electron transfer event, thereby repeatedly engaging in activation of neighboring activatable molecules. In some embodiments, a photocatalyst that, upon absorption of light at an appropriate wavelength, is capable of engaging in energy transfer events with oxygen to generate reactive species (e.g., a proton, singlet oxygen, etc.), is referred to as a “photosensitizer.” Some embodiments herein described in conjunction with a photocatalyst may encompass or be limited to a photosensitizer.

As used herein, the term “small molecule” refers to a low molecular weight (e.g., <2000 daltons, <1000 daltons, <500 daltons) organic compound, with dimensions (e.g., length, width, diameter, etc.) on the order of 1 nm. Larger structures, such as peptides, proteins, and nucleic acids, are not small molecules, although their constituent monomers (ribo-or deoxyribonucleotides, amino acids, etc.) are considered small molecules.

As used herein, the term “cell permeable” refers to a compound or moiety that is capable of effectively crossing a cell membrane that has not been synthetically permeabilized.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; Carruthers, Some Modern Methods of Organic Synthesis, 3^{rd} Edition, Cambridge University Press, Cambridge, 1987; the entire contents of each of which are incorporated herein by reference.

As used herein, the term “physiological conditions” encompasses any conditions compatible with living cells, e.g., predominantly aqueous conditions of a temperature, pH, salinity, chemical makeup, etc. that are compatible with living cells.

As used herein, the terms “conjugated” and “conjugation” refer to the covalent attachment of two molecular entities (e.g., post-synthesis and/or during synthetic production). Conjugated entities may be peptides or proteins that are “fused” by a peptide linkage, or may also include other molecular entities (e.g., nucleic acid, small molecules, etc.) connected directly or by suitable linkers.

The term “binding moiety” refers to a domain that specifically binds an antigen or epitope independently of a different epitope or antigen binding domain. A binding moiety may be an antibody, antibody fragment, a receptor domain that binds a target ligand, proteins that bind to immunoglobulins (e.g., protein A, protein G, protein A/G, protein L, protein M), a binding domain of a proteins that bind to immunoglobulins (e.g., protein A, protein G, protein A/G, protein L, protein M), oligonucleotide probe, peptide nucleic acid, DARPin, anticalin, nanobody, aptamer, affimer, a purified protein (either the analyte itself or a protein that binds to the analyte), and analyte binding domain(s) of proteins etc. Table A provides a list of exemplary binding moieties that could be used singly or in various combinations in methods, systems, and assays (e.g., immunoassays) herein.

TABLE A
Exemplary binding moieties
Protein A
Ig Binding domain of protein A
Protein G
Ig Binding domain of protein G
Protein L
Ig Binding domain of protein L
Protein M
Ig Binding domain of protein M
polyclonal antibody against analyte X
monoclonal antibody
recombinant antibody
scFv
variable light chain (V_{L}) of antibody (monoclonal, recombinant,
polyclonal) recognizing target analyte X
protein (e.g., receptor) binding domain that binds to analyte X
(Fab) fragment
Fab′ fragment
Fv fragment
F(ab′)2 fragment
oligonucleotide probe
DARPins and other synthetic binding scaffolds (ex: Bicycles)
peptide nucleic acid
aptamer
affimer

As used herein, the term “antibody” refers to a whole antibody molecule or a fragment thereof (e.g., fragments such as Fab, Fab′, and F (ab′)₂, variable light chain, variable heavy chain, Fv). It may be a polyclonal or monoclonal or recombinant antibody, a chimeric antibody, a humanized antibody, a human antibody, etc. As used herein, when an antibody or other entity “specifically recognizes” or “specifically binds” an antigen or epitope, it preferentially recognizes the antigen in a complex mixture of proteins and/or macromolecules and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (K^{a}) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “antibody fragment” refers to a portion of a full-length antibody, including at least a portion of the antigen binding region or a variable region. Antibody fragments include, but are not limited to, Fab, Fab′, F (ab′)₂, Fv, scFv, Fd, variable light chain, variable heavy chain, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis. For example, a “Fab” fragment comprises one light chain and the CHI and variable region of one heavy chain. The heavy chain of a Fab molecule cannot form a disulfide bond with another heavy chain molecule. A “Fab”' fragment comprises one light chain and one heavy chain that comprises an additional constant region extending between the C_{H1} and C_{H2} domains. An interchain disulfide bond can be formed between two heavy chains of a Fab′ fragment to form a “F(ab′)₂” molecule. An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen. Other antibody fragments will be understood by skilled artisans.

As used herein, the term “biomolecule” or “biological molecule” refers to molecules and ions that are present in organisms and are essential to a biological process(es) such as cell division, morphogenesis, or development. Biomolecules include large macromolecules (or polyanions) such as proteins, carbohydrates, lipids, and nucleic acids as well as small molecules such as primary metabolites, secondary metabolites, and natural products. A more general name for this class of material is biological materials. Biomolecules are usually endogenous, but may also be exogenous. For example, pharmaceutical drugs may be natural products or semisynthetic (biopharmaceuticals), or they may be totally synthetic.

As used herein, the term “alkyl” means a straight or branched saturated hydrocarbon chain containing from 1 to 30 carbon atoms, for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-Cs alkyl), 1 to 6 carbon atoms (C₁-C₆ alkyl), 1 to 4 carbon atoms (C₁-C₄ alkyl), 6 to 20 carbon atoms (C₆-C₂₀ alkyl), or 8 to 14 carbon atoms (C₈-C₁₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “amino” means a —NH₂ group.

As used herein, the term “haloalkyl” means an alkyl group, as defined herein, in which at least one hydrogen atom (e.g., one, two, three, four, five, six, seven or eight hydrogen atoms) is replaced by a halogen.

As used herein, the term “heteroalkyl” means an alkyl group, as defined herein, in which one or more of the carbon atoms (and any associated hydrogen atoms) are each independently replaced with a heteroatom group such as —NR—, —O—, —S—, —S(O)—, —S(O)₂—, and the like, where R is H, alkyl, aryl, cycloalkyl, heteroalkyl, heteroaryl, or heterocyclyl, each of which may be optionally substituted. By way of example, 1, 2, or 3 carbon atoms may be independently replaced with the same or different heteroatomic group. Examples of heteroalkyl groups include, but are not limited to, —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. Heteroalkyl also includes groups in which a carbon atom of the alkyl is oxidized (i.e., is —C(O)—).

### DETAILED DESCRIPTION

Provided herein are systems, methods, and compositions for bioluminescence-triggered photocatalytic activation of molecular entities in a proximity dependent manner, which can be actuated within biological systems. In particular, provided herein are bioluminescent proteins or complexes, luminophore substrates thereof, photocatalysts, and activatable molecular entities incorporating light-responsive moieties that restrict their activity; systems thereof; and methods for catalytically activating the activatable molecular entities via bioluminescence-triggered catalysis.

The need to study dynamic microenvironments, signaling pathways, and molecular processes in physiologically relevant contexts presents a demand for new functional biology tools enabling such analyses in live cells and complex models in a nondestructive fashion. Bioluminescence-triggered catalytic activation of molecular entities in a proximity-dependent manner offers a solution to this need by utilizing a non-invasive, intrinsic light-source to activate molecular entities (e.g., molecules incorporating light-sensitive moieties that restrict their activity including photolabile protecting groups (photocages), photoswitches, etc.) in biological systems for subsequent activities (e.g., detection, interactions with biomacromolecules etc.). The components for such photocatalytic systems include a bioluminescent light source (e.g., luciferase (e.g., NanoLuc) or bioluminescent complex (e.g., NanoBiT, NanoTrip, etc.) and pairs of (1) light-sensitive catalyst (transition metal or organic dye catalyst) and (2) activatable molecules. Upon luminophore substrate addition, the bioluminescent entity (e.g., NanoBiT, NanoTrip, NanoLuc, etc.) generates light that triggers local photocatalytic activation of the activatable molecule. These activated molecules can be leveraged for a broad range of spatiotemporally controlled phenotypic, proteomic, and genomic analyses including detection, activation, inactivation, and degradation of proximal proteins and nucleic acids as well as probing and altering of biological processes.

Advantages of using bioluminescence as the light source rather than global-light radiation (e.g., LED or laser) include: using an intrinsic light source that is mild and minimally destructive; reduced phototoxicity; efficient light delivery for triggering catalysis in intact cells and complex models; local and conditional (+substrate) delivery of light for greater spatiotemporal resolution over catalyst activation and downstream chemistries; and the ability to tether the light source to target molecules and/or other components of the system (e.g., the photocatalyst).

In some embodiments, provided herein are systems comprising one or more of a bioluminescent protein or structurally-complementary components of a bioluminescent complex; a luminophore, wherein the bioluminescent protein catalyzes emission of a first wavelength of light from the luminophore upon interaction therewith; a photocatalyst, wherein the photocatalyst is activated upon absorption of light of the first wavelength; and an activatable molecule, wherein the activatable molecule is converted into an activated molecule when in proximity to the activated photocatalyst.

In some embodiments, a bioluminescent protein or component of a bioluminescent complex is linked to a photocatalyst. In some embodiments, the linkage of the photocatalyst to the light source provides the appropriate proximity for activating the photocatalyst.

### Bioluminescent Protein or Complex

The present disclosure includes materials and methods related to bioluminescent polypeptides, bioluminescent complexes, and components thereof. In particular, light emitted from bioluminescent proteins or complexes (or from luminophores acted upon by bioluminescent proteins or complexes) is used to activate photocatalysts. NanoLuc

In some embodiments, systems and methods herein comprise a bioluminescent protein. In some embodiments, a bioluminescent protein is a luciferase enzyme. Suitable luciferase enzymes include those selected from the group consisting of: Photinus pyralis or North American firefly luciferase; Luciola cruciata or Japanese firefly or Genji-botaru luciferase; Luciola italic or Italian firefly luciferase; Luciola lateralis or Japanese firefly or Heike luciferase; N. nambi luciferase; Luciola mingrelica or East European firefly luciferase; Photuris pennsylvanica or Pennsylvania firefly luciferase; Pyrophorus plagiophthalamus or Click beetle luciferase; Phrixothrix hirtus or Railroad worm luciferase; Renilla reniformis or wild-type Renilla luciferase; Renilla reniformis Rluc8 mutant Renilla luciferase; Renilla reniformis Green Renilla luciferase; Gaussia princeps wild-type Gaussia luciferase; Gaussia princeps Gaussia-Dura luciferase; Cypridina noctiluca or Cypridina luciferase; Cypridina hilgendorfii or Cypridina or Vargula luciferase; Metridia longa or Metridia luciferase; TurboLuc (Auld et al. Biochemistry 2018, 57, 31, 4700-4706: incorporated by reference in its entirety); Nano-lanterns (Suzuki et al. Nature Communications volume 7, Article number: 13718 (2016); incorporated by reference in its entirety); and Oplophorus luciferase (e.g., Oplophorus gracilirostris (OgLuc luciferase), Oplophorus grimaldii, Oplophorus spinicauda, Oplophorus foliaceus, Oplophorus noraczeelandiae, Oplophorus typus, Oplophorus noraezelandiae or Oplophorus spinous).

In some embodiments, the bioluminescent protein is a luciferase of Oplophorus gracilirostris, the NanoLuc® luciferase (Promega Corporation; U.S. Pat. No. 8,557,970; U.S. Pat. No. 8,669,103; herein incorporated by reference in their entireties). PCT Appln. No. PCT/US2010/033449, U.S. Pat. No. 8,557,970, PCT Appln. No. PCT/2011/059018, and U.S. Pat. No. 8,669,103 (each of which is herein incorporated by reference in their entirety and for all purposes) describe compositions and methods comprising bioluminescent polypeptides. Such polypeptides find use in embodiments herein and can be used in conjunction with the compositions, assays, devices, systems, and methods described herein. In some embodiments, compositions, assays, devices, systems, and methods provided herein comprise a bioluminescent polypeptide of SEQ ID NO: 1, or having at least 60% (e.g., 60%, 65%, 70%, 75%, 80%, 85%, 90%, 95%, 96%, 97%, 98%, 99%, 100%, or ranges therebetween) sequence identity with SEQ ID NO: 1. In some embodiments, any of the aforementioned bioluminescent proteins are linked (e.g., fused, chemically linked, etc.) to one or more other components of the assays and systems described herein (e.g., fused to a HALOTAG protein).

In some embodiments, a bioluminescent protein is a circularly permuted version of a natural or modified bioluminescent protein (See, e.g., U.S. Pat. No. 10,774,364; incorporated by reference in its entirety).

In some embodiments, systems and methods herein comprise a bioluminescent complex (e.g., two or more components (e.g., peptides and/or polypeptides) that combine through structural complementation to form a complex that is capable of activating a luminophore to emit light). In some embodiments, a luminophore emits significantly more light in the presence of the bioluminescent complex than in the presence of any one of the components alone). In some embodiments, a bioluminescent complex is formed from fragments (e.g., peptide(s) and/or polypeptide(s)) of a luciferase enzyme. In some embodiments, a bioluminescent complex is a circularly permuted version of a natural or modified bioluminescent component (e.g., formed from two fragments of a circularly permuted luciferase); See, e.g., U.S. Pat. No. 10,774,364; incorporated by reference in its entirety.

PCT Appln. Nos. PCT/US14/26354, PCT/US19/036844, and PCT/US20/62499; U.S. Pat. No. 9,797,889; U.S. Pat. Appln. Ser. No. 16/439,565; and U.S. Pub. No. 2021/0262941 (each of which is herein incorporated by reference in their entirety and for all purposes) describe compositions and methods for the assembly of bioluminescent complexes; such complexes, and the peptide and polypeptide components thereof, find use in embodiments herein and can be used in conjunction with the assays and methods described herein.

In some embodiments, peptide and polypeptide components are provided for the assembly of a bioluminescent complex, capable of generating luminescence in the presence of an appropriate substrate (e.g., a coelenterazine or a coelenterazine analog (e.g., furimazine, fluorofurimazine, etc.). In some embodiments, complementary polypeptide(s) and peptide(s) collectively span the length (or >75% of the length, >80% of the length, >85% of the length, >90% of the length, >95% of the length, or more) of a luciferase base sequence (or collectively comprise at least 40% sequence identity to a luciferase base sequence (e.g., >40%, >45%, >50%, >55%, >60%, >65%, >70%, >75% >80%, >85%, >90%, >95%, or more). In some embodiments, “complementary” polypeptide(s) and peptide(s) are separate molecules that each correspond to a portion of a luciferase base sequence. Through structural complementarity, they assemble to form a bioluminescent complex. Suitable luciferase base sequences may include SEQ ID NOS: 1 or 2, or the sequences of any of the full-length luciferases listed above. In some embodiments, the bioluminescent complex comprises the NANOBIT or NANOTRIP systems (Promega; Madison, WI). In some embodiments, the peptide and/or polypeptide components of a bioluminescent complex collectively comprise at least 60% sequence identity (e.g., >60%, >65%, >70%, >75%, >80%, >85%, >90%, >95%, >99%) with SEQ ID NO: 1 and/or SEQ ID NO: 2. In some embodiments, the peptide and/or polypeptide components of the bioluminescent complex comprise HIBIT (SEQ ID NO: 3), SMBIT (SEQ ID NO: 4), LGBIT (SEQ ID NO: 5), LGTRIP (SEQ ID NO: 6), and/or SMTRIP9 (SEQ ID NO: 7). In some embodiments, the peptide and/or polypeptide components of the bioluminescent complex comprise at least 60% sequence identity (e.g., >60%, >65%, >70%, >75%, >80%, >85%, >90%, >95%, >99%) with HIBIT (SEQ ID NO: 3), SMBIT (SEQ ID NO: 4), LGBIT (SEQ ID NO: 5), LGTRIP (SEQ ID NO: 6), and/or SMTRIP9 (SEQ ID NO: 7).

In some embodiments, any of the aforementioned components of bioluminescent complexes are linked (e.g., fused, chemically linked, tethered, etc.) to one or more other components of the assays and systems described herein (e.g., fused to a HALOTAG protein).

There are various characteristics of the bioluminescent complexes that find use in embodiments herein that may provide advantages in certain applications. For example, a bioluminescent complex (e.g., a complex formed upon complementation of HiBiT/LgBiT) only generates light upon complementation of its component peptide/polypeptides; therefore, directly or indirectly conjugating (e.g., fusing, tethering, etc.) one or more components of the bioluminescent complex to other components of the system (e.g., photocatalyst, activatable molecule, target, etc.) ensuring the proximity of that component to the bioluminescent complex upon light generation. Tethering of two other components of the system to separate components of the bioluminescent complex ensures the proximity of those components upon light generation by the complex. In some embodiments, the use of a bioluminescent complex, due to the requirement that two components come together to form the complex, provides enhanced spatiotemporal resolution through conditional activation at a specific site.

In some embodiments the bioluminescent protein or a component of the multipart bioluminescent complex is inserted in an internal position within the capture agent. In some embodiments, a position within the capture agent is selected to increase efficiency of bioluminescence activation of the catalyst through greater proximity or favorable conformation.

In some embodiments, the bioluminescent protein or a component of the multipart bioluminescent complex is circularly permuted.

### Luminophore Substrate

In some embodiments, the systems and methods herein comprise luminophore substrates that emit light upon interaction with the bioluminescent proteins and/or complexes described herein. Suitable luminophores for the bioluminescent protein or complex used in the system or method will be understood. For example, firefly luciferin, with the structure:

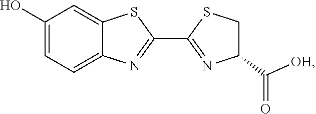 [SMILES: O=C(O)[C@H]1CSC(c2nc3ccc(O)cc3s2)=N1]

is the luciferin found in many Lampyridae species, and is the substrate of beetle luciferases.

Latia luciferin, with the structure:

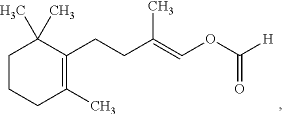 [SMILES: CC1=C(CC/C(C)=C/OC=O)C(C)(C)CCC1 ; ","]

is from the freshwater snail Latia neritoides.

Bacterial luciferin, with the structure:

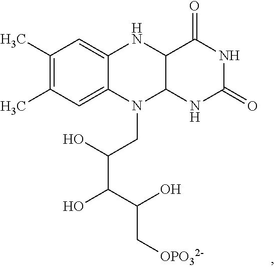

finds use as a substrate for many bacterial luciferases.

Coelenterazine of the structure:

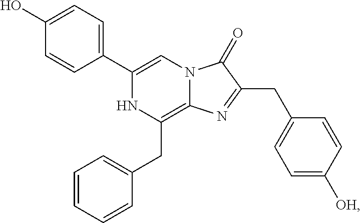 [SMILES: O=c1c(Cc2ccc(O)cc2)nc2c(Cc3ccccc3)[nH]c(-c3ccc(O)cc3)cn1-2]

is found in radiolarians, ctenophores, cnidarians, squid, brittle stars, copepods, chaetognaths, fish, and shrimp, and is the luminophore substrate for the luciferases of those organisms. Variants and derivatives of coelenterazine, such as furimazine and fluorofurimazine find use in embodiments herein (e.g., with Oplophorus-derived bioluminescent proteins and complexes).

Other luminophore substrates include those of dinoflagellates:

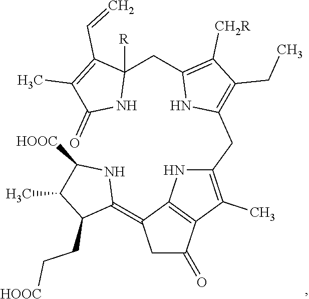

Vargulin (cypridin luciferin):

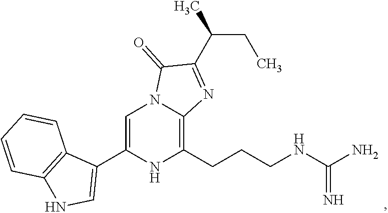 [SMILES: CC[C@H](C)c1nc2c(CCCNC(=N)N)[nH]c(-c3c[nH]c4ccccc34)cn-2c1=O]

and N. nambi:

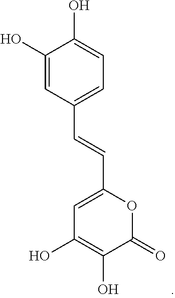 [SMILES: O=c1oc(/C=C/c2ccc(O)c(O)c2)cc(O)c1O]

Pairing of appropriate bioluminescent proteins or complexes with luminophores is understood in the field. In particular embodiments, a bioluminescent protein is provided in a system or method herein that utilizes an imidazopyrazine luminophore, such as coelenterazine, furimazine, or fluorofurimazine (U.S. application Ser. No. 16/548,214; incorporated by reference in its entirety). In some embodiments, a system or method comprises (1) an Oplophorus-derived polypeptide (e.g., NANOLUC) or components of an Oplophorus-derived bioluminescent complex (e.g., NANOBIT, NANOTRIP) and an imidazopyrazine luminophore (e.g., coelenterazine, furimazine, fluorofurimazine, etc.). In some embodiments, systems and methods herein comprise an imidazopyrazine luminophore such as native coelenterazine, furimazine, fluorofurimazine, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); and U.S. Pat. No. 8,669,103; the disclosures of which are incorporated by reference herein in their entireties.

In some embodiments, the luminophore emits light upon interaction with the bioluminescent protein or complex. In some embodiments, the luminophore emits light in the visible light spectrum (e.g., about 400 to about 700 nm (e.g., 400 nm, 425 nm, 450 nm, 475 nm, 500 nm, 525 nm, 550 nm, 575 nm, 600 nm, 625 nm, 650 nm, 675 nm, 700 nm, or ranges therebetween). In some embodiments, the luminophore emits light of a wavelength between 400 and 500 nm (e.g., 400 nm, 410 nm, 420 nm, 430 nm, 440 nm, 450 nm, 460 nm, 470 nm, 480 nm, 490 nm, 500 nm, or ranges therebetween).

### Photocatalysts

In some embodiments, the systems and methods herein comprise a photocatalyst that is capable of absorbing light emitted from a luminophore (upon interaction with a bioluminescent protein or complex) and subsequently activating a neighboring activatable molecule. Any compound or moiety capable of receiving light energy emitted from a bioluminescent protein-or complex-activated luminophore and subsequently engaging in activation of an activatable molecule may find use in embodiments herein. In some embodiments, the excited photocatalyst engage in activation of neighboring activatable molecule via Förster Resonance Energy Transfer, Dexter Energy Transfer, Single Electron Transfer, or any other suitable mechanism of energy or electron transfer in activation of neighboring activatable molecules via generation of singlet oxygen and abstraction of a hydrogen from the activatable molecule (direct oxidation).

In some embodiments, the photocatalyst is an iridium-based or ruthenium-based photocatalyst (Bevernaegie et al. ‘A Roadmap Towards Visible Light Mediated Electron Transfer Chemistry with Iridium (III) Complexes.’ ChemPhotoChem 2021, 5, 217.; Day et al. Advances in Photocatalysis: A Microreview of Visible Light Mediated Ruthenium and Iridium Catalyzed Organic Transformations Org. Process Res. Dev. 2016, 20, 1156-1163; incorporated by reference in their entireties). In some embodiments, the photocatalyst is of the structure of Formula (I):

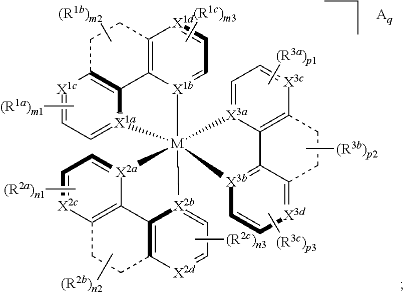

wherein:

each set of dashed lines (------) represents the presence or absence of a fused 6-membered ring;

M is a transition metal;

m1, m2, m3, n1, n2, n3, p1, p2, and p3 are each independently 0, 1, or 2;

R^{1a}, R^{1b}, R^{1C}, R^{2a}, R^{2b}, R^{2c}, R^{3a}, R^{3b}, and R^{3c} are each independently selected from halo, alkyl, haloalkyl, amino, heteroalkyl, and a group-Linker-Q, wherein Q is a capture element;

X^{1a}, X^{1b}, X^{2a}, X^{2b}, X^{3a}, and X^{3b} are each independently selected from N and C, wherein at least one of X^{1a} and X^{1b} is N, at least one of X^{2a} and X^{2b} is N, and at least one of X^{3a} and X^{3b} is N;

X^{1c}, X^{1d}, X^{2c}, X^{2d}, X^{3c}, and X^{3d} are each independently selected from CH and N; A is an anion; and

q is 0, 1, or 2.

In some embodiments, the photocatalyst comprises a transition metal selected from Ru and Ir.

In some embodiments, the photocatalyst is an iridium-based photocatalyst selected from:

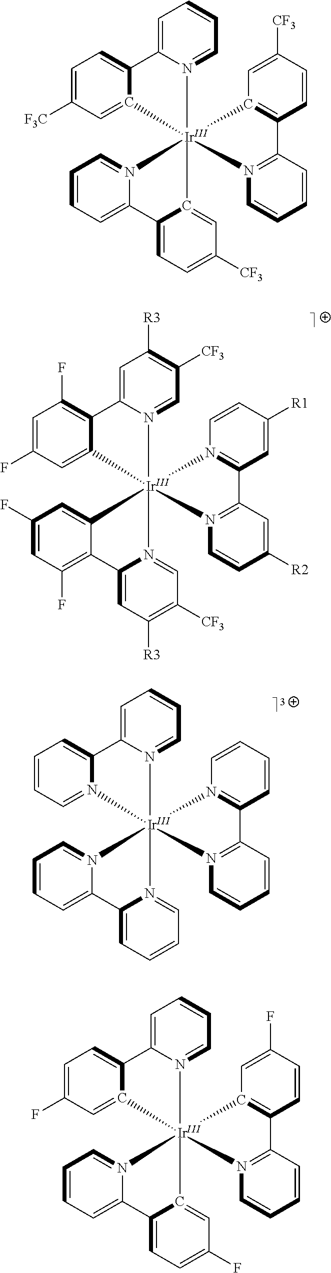

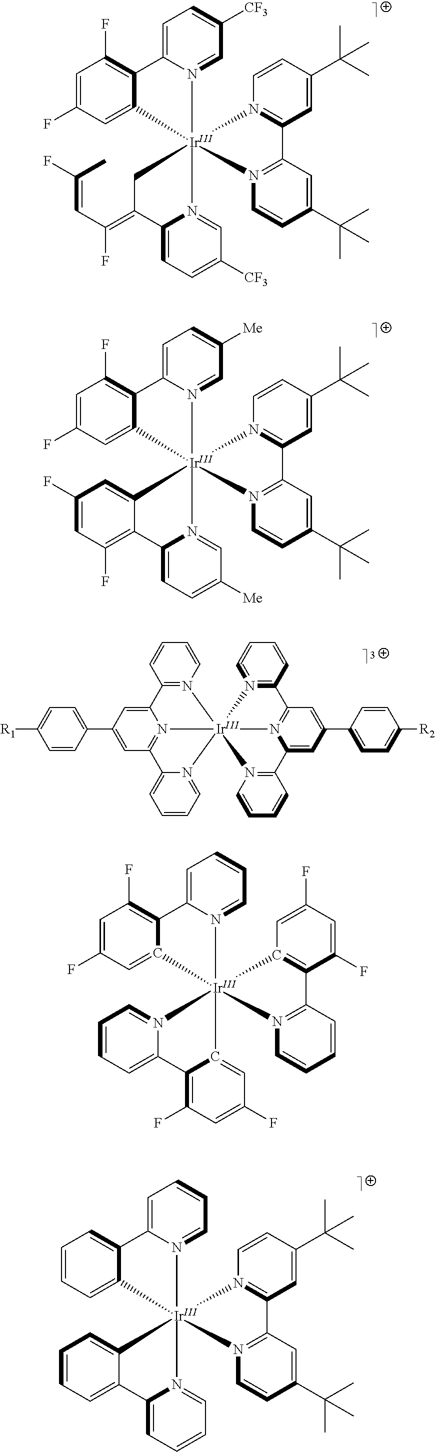

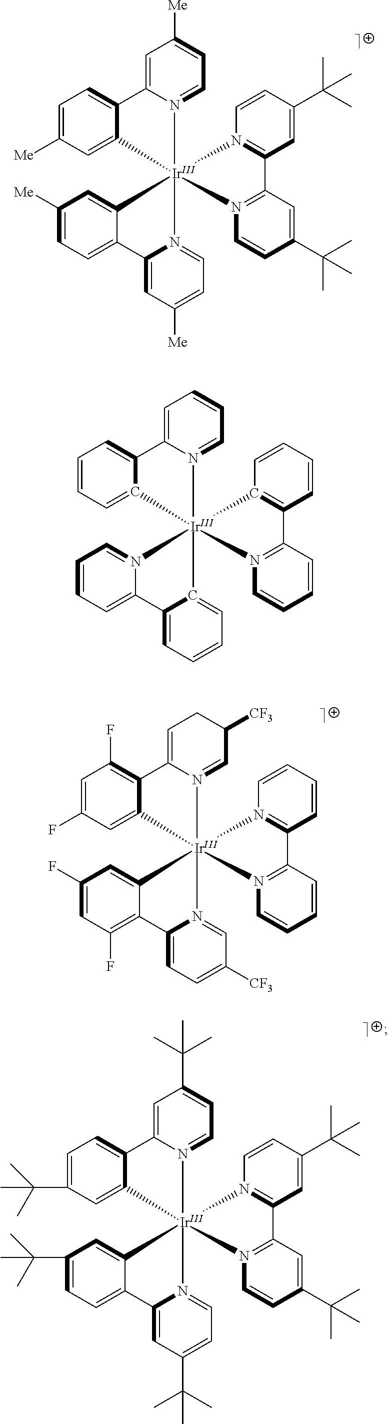

or a derivative thereof in which the compound is functionalized with at least one group-Linker-Q (wherein Q is a capture clement).

In some embodiments, the photocatalyst is a ruthenium-based photocatalyst selected from:

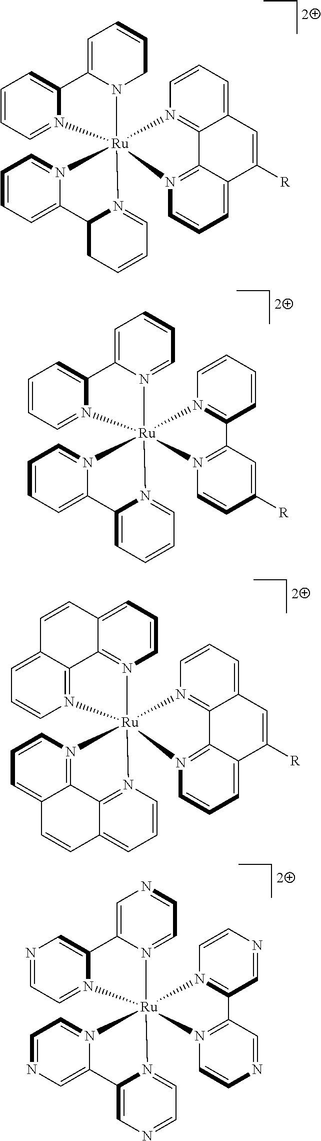

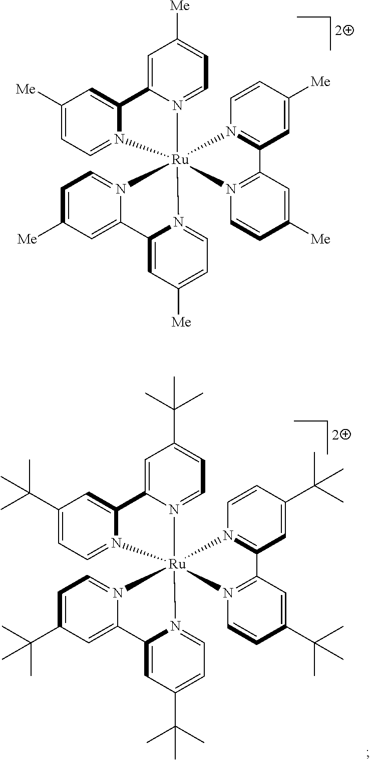

or a derivative thereof in which the compound is functionalized with at least one group-Linker-Q (wherein Q is a capture clement).

In some embodiments, M is Ru. In some embodiments, M is Ir.

In some embodiments, m2, n2, and p2 are each 0 and each set of dashed lines represents the absence of a fused 6-membered ring, i.e., the compound has formula:

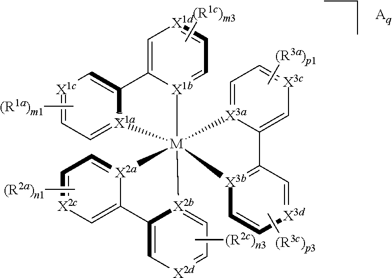

In some embodiments, X^{1a} is N, X^{1b} is C, X^{2a} is N, X^{2b} is C, X^{3a} is C, and X^{3b} is N. In some embodiments, X^{1a} is N, X^{1b} is C, X^{2a} is N, X^{2b} is C, X^{3a} is N, and X^{3b} is N.

In some embodiments, X^{1c}, X^{1d}, X^{2c}, X^{2d}, X^{3c}, and X^{3d} are each CH. In some embodiments, X^{1c}, X^{1d}, X^{2c}, X^{2d}, X^{3c}, and X^{3d} are each N. In some embodiments, R^{1a}, R^{1b}, R^{1c}, R^{2a}, R^{2b}, R^{2c}, R^{3a}, R^{3b}, and R^{3c} are each independently selected from fluoro, methyl, tert-butyl, trifluoromethyl, and a group-Linker-Q. In some embodiments, no more than one of R^{1a}, R^{1b}, R^{1c}, R^{2a}, R^{2b}, R^{2c}, R^{3a}, R^{3b}, and R^{3c} is a group-Linker-Q.

In some embodiments, the compound comprises one group “-Linker-Q,” wherein Q is a capture element. In some embodiments, a capture element is an “affinity molecule,” and the corresponding capture agent is an “acceptor” (e.g., small molecule, protein, antibody, etc.) that selectively interacts with the affinity molecule. Examples of such pairs would include: an antigen as the capture element and an antibody as the capture agent, a small molecule as the capture element and a protein with high affinity for the small molecule as the capture agent (e.g., streptavidin and biotin), and the like.

In some embodiments, Q is a substrate for a dehalogenase, e.g., a haloalkane dehalogenase. Systems comprising mutant hydrolases (e.g., mutant dehalogenases) that covalently bind their substrates (e.g., haloalkyl substrates) are described, for example, in U.S. Pat. Nos. 7,238,842; 7,425,436; 7,429,472; 7,867,726; each of which is herein incorporated by reference in its entirety. For example, HALOTAG is a commercially-available modified dehalogenase enzyme that forms a stable (e.g., covalent) bond (e.g., ester bond) with its haloalkyl substrate, which finds use in embodiments herein.

In some embodiments, Q has a formula —(CH₂)ₙ—Y, wherein n is 2, 3, 4, 5, 6, 7, 8, 9, 10, 11, or 12, and Y is a halogen (i.e., F, Cl, Br, or I). In some embodiments, n is 4, 5, 6, 7, or 8, and Y is Cl. In some embodiments, n is 6 and Y is Cl, such that Q has formula —(CH₂)₆—Cl.

The Linker may include various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo-and poly-ethylene glycol (—(CH₂CH₂O)ₓ—) linkages, and the like. In some embodiments, the linker may include 2 or more atoms (e.g., 2-200 atoms, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 atoms, or any range therebetween (e.g., 2-20, 5-10, 15-35, 25-100, etc.)). In some embodiments, the linker includes a combination of oligoethylene glycol linkages and carbamate linkages. In some embodiments, the linker has a formula —O(CH₂CH₂O)_{z1}—C(O)NH—(CH₂CH₂O)_{z2}—C(O)NH—(CH₂)_{z3}—(OCH₂CH₂)_{z4}O—, wherein z1, z2, z3, and z4 are each independently selected form 0, 1, 2, 3, 4, 5, and 6. For example, in some embodiments, the linker has a formula selected from:

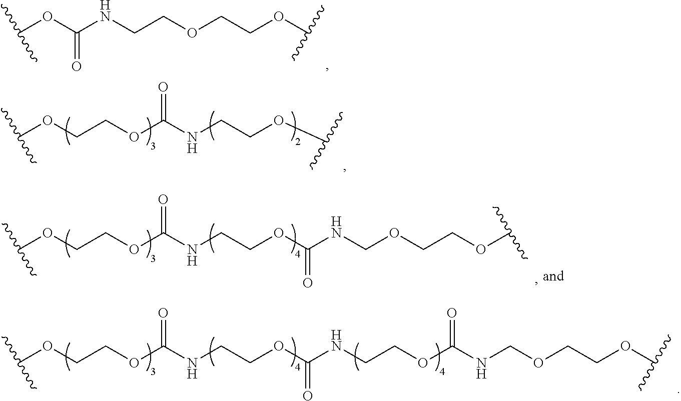

In some embodiments, q is 0, 1, or 2. Those skilled in the art will recognize that the value of q will depend on the selection of other variables and will be selected to balance the overall charge on the rest of the molecule. For example, if the overall charge of the metal-based portion of the molecule is +1, then in some embodiments, q is 1 and A is a monovalent anion (e.g., a halide or hexafluorophosphate). In some embodiments, the overall charge of the metal-based portion of the molecule is +2, then in some embodiments, q is 2 and A is a monovalent anion (e.g., a halide or hexafluorophosphate).

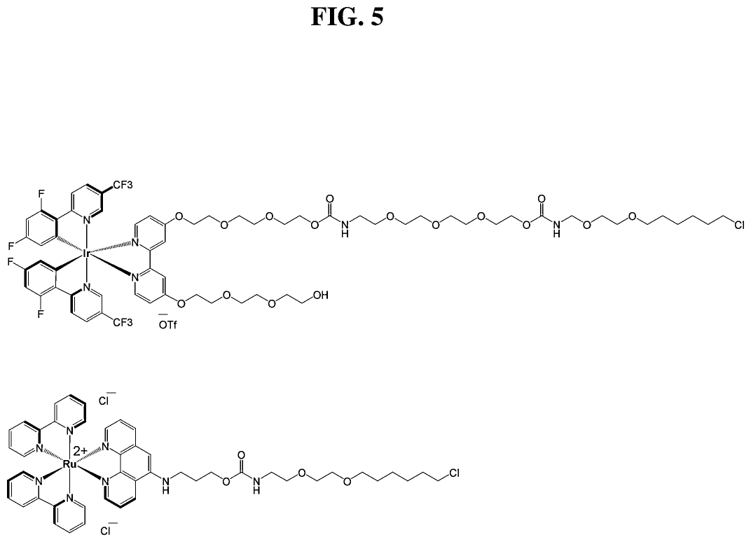

An exemplary photocatalyst linked to a HALOTAG substrate is depicted in FIG. 5. Alternative positions for attachment to the photocatalyst, other photocatalysts, different linkers and linker lengths, etc., will be understood to be within the scope herein.

In some embodiments, the photocatalyst is an organic photoredox catalyst. In some embodiments, the organic photoredox catalyst is selected from a quinone, a pyrylium, an acridinium, and a xanthene.

In some embodiments, the photocatalyst is quinone-based organic photoredox catalyst selected from:

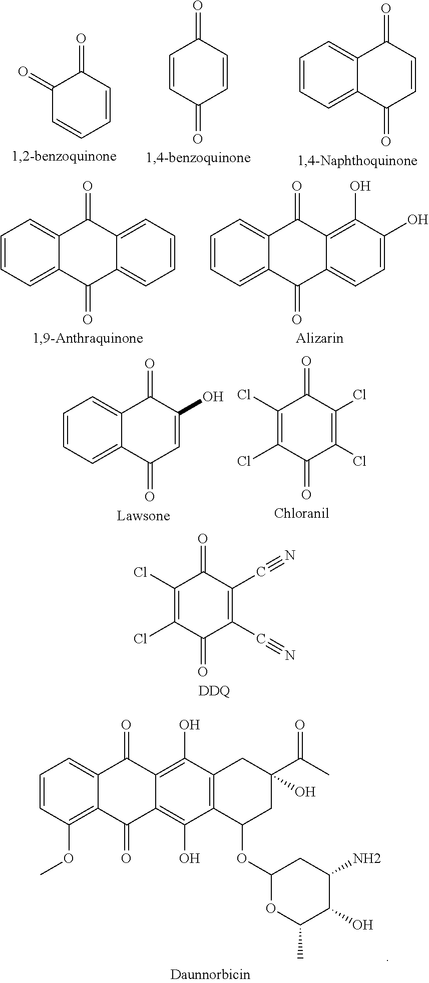

In some embodiments, the photocatalyst is pyrylium-based organic photoredox catalyst selected from:

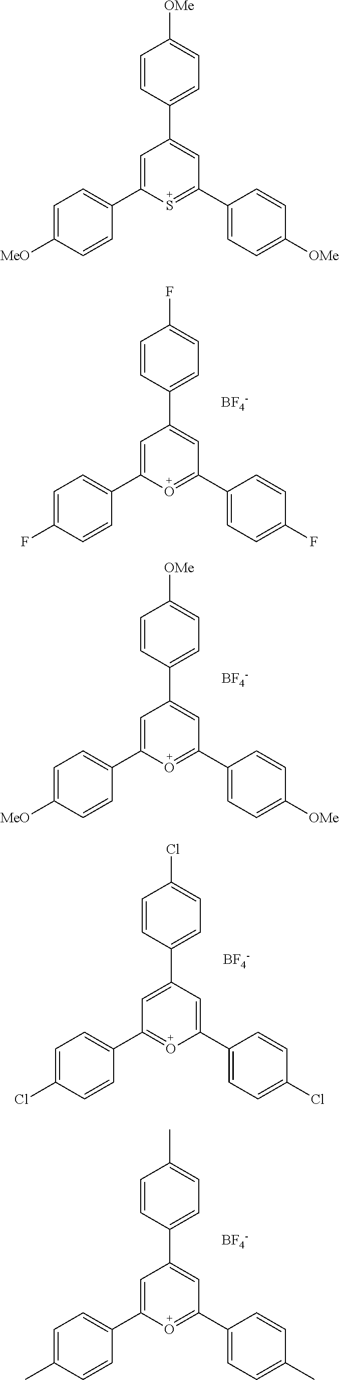

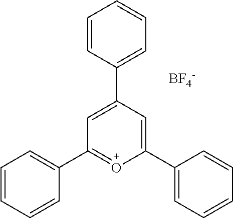

In some embodiments, the photocatalyst is acridinium-based organic photoredox catalyst selected from:

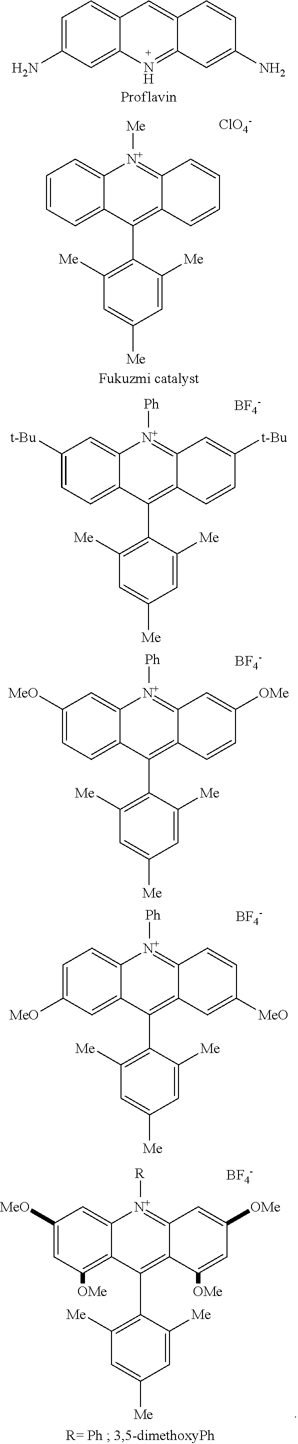

In some embodiments. the photocatalyst is xanthene-based organic photoredox catalyst selected from:

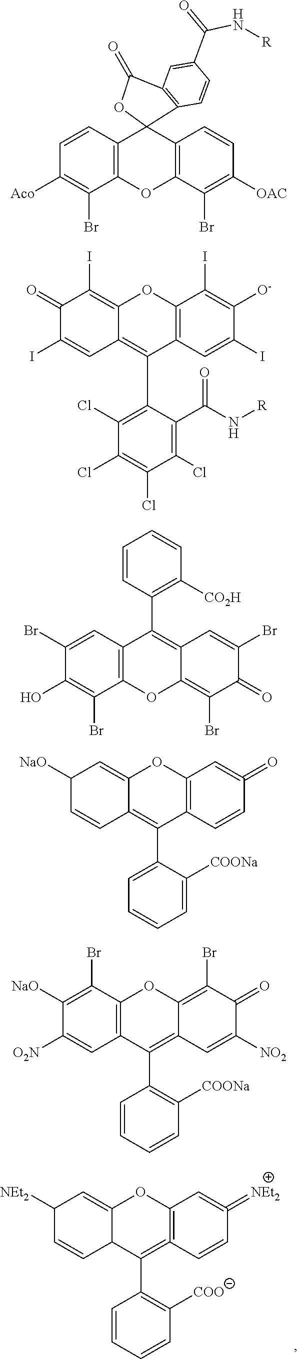

wherein R, when present represents a potential attachment site for -Linker-Q.

In some embodiments, any suitable positions in the above photocatalyst structures may find use as an attachment site for Linker-Q.

In some embodiments, the photocatalyst is thiazine-based organic photoredox catalyst selected from:

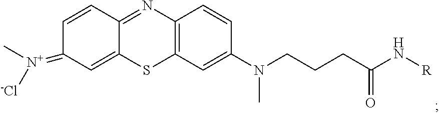

wherein R is an attachment site for Linker-Q. In some embodiments, R is an amine, a carboxyl, tert-butyl, tert-butyl-methoxy, ether, hydroxyl, PEG, etc.

In some embodiments, a photocatalyst (e.g., a quinone-based, pyrylium-based, acridinium-based, xanthene-based, or thiazine-based photoredox catalyst) is conjugated to a Linker-Q. In some embodiments, a linker (e.g., Linker-Q) is attached to the photocatalyst at any suitable position on the photocatalyst structures. In some embodiments, positions suitable for attachment of the photocatalysts are understood in the field.

### Activatable Molecules

In some embodiments, systems and methods herein comprise activatable molecules incorporating light responsive moieties that restrict their activity, which when acted upon by an activated photocatalyst are converted from an activatable molecule to an activated molecule. In some embodiments, the photocatalyst catalyzes bond cleavage on the activatable molecule (releasing the activatable molecule from another entity, releasing a moiety from the activatable molecule, etc.). In some embodiments, the photocatalyst catalyzes oxidation (i.e., abstraction of a hydrogen) of the activatable molecules, which either turn-on its reactivity or releases it from another entity. In some embodiments, the photocatalyst catalyzes a reversible conformational change that turn-on the reactivity of the activatable molecules. In some embodiments, the photocatalyst catalyzes the formation of a bond to the activatable molecule (e.g., attaching the activatable molecule to another entity. Embodiments herein are not limited by the mechanism of chemistry of molecular activation.

In some embodiments, the photocatalyst facilitates energy transfer to the activatable molecule. In some embodiments, the photocatalyst transfers energy to the activatable molecule by Förster Resonance Energy Transfer, Dexter Energy Transfer, Single Electron Transfer, or any other suitable mechanism of energy transfer. In some embodiments, the photocatalyst generates singlet oxygen for direct oxidation of the activatable molecule.

In some embodiments, the activatable molecule is a caged compound. Caged compounds are activatable molecules that have been rendered inert (e.g., chemically inert, biologically inert, undetectable, etc.) by a chemical modification. In some embodiments, conversion of the activatable molecule into the activated molecule comprises uncaging the activatable molecule. In some embodiments, an activated photocatalyst facilitates the uncaging of the activatable molecule, resulting in liberation of the active from of the molecule. Embodiments herein are not limited by the identity of the caged molecule, the caging modification, or the chemistry required for uncaging. For example, the activatable molecule may comprise, a photocaged fluorophore, a photocaged probe, a photocaged drug, a photocaged signaling molecule, a photocaged neurotransmitter, a photocaged crosslinker, a photocaged proteolysis targeting chimera (PROTAC), a photocaged gRNA, a photocaged nucleic acid (RNA or DNA), a photocaged nucleotide, etc. In some embodiments, the caged molecule is a small molecule, a peptide, or a nucleic acid.

Examples for photolabile protecting groups and bond cleavage they undergo to release activated molecules include:

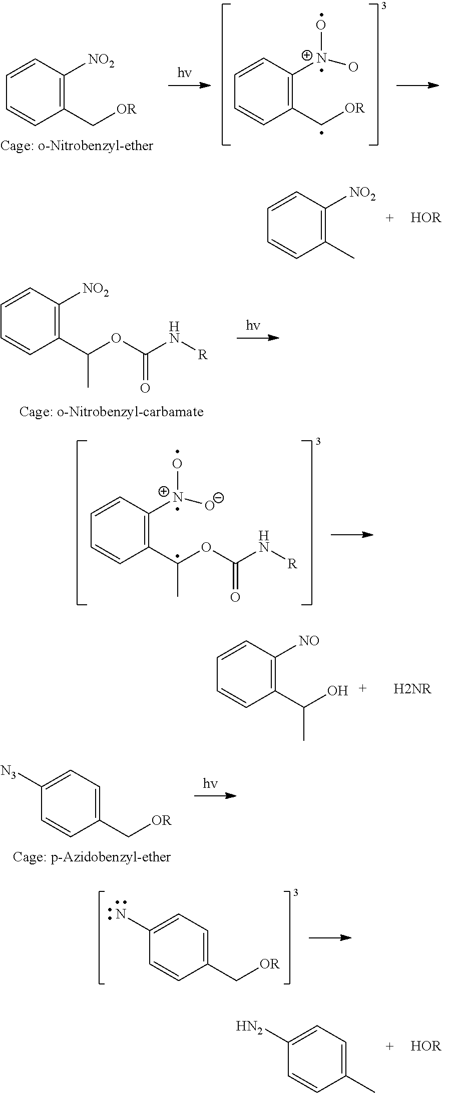

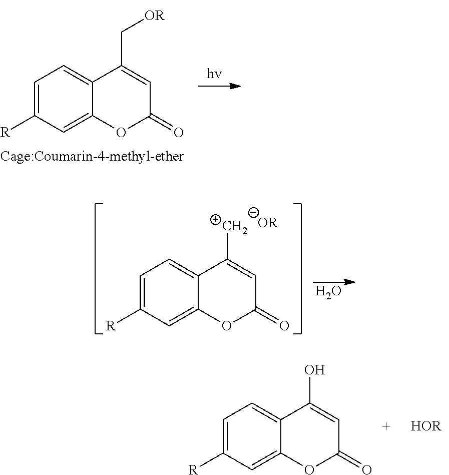

Other photolabile protecting chemistries find use in embodiments herein (Klán et al. Photoremovable Protecting Groups in Chemistry and Biology: Reaction Mechanisms and Efficacy Chem. Rev. 2013, 113, 119-191; incorporated by reference in its entirety).

In some embodiments, the photocatalyst facilitates abstraction of a hydrogen from the activatable molecule. Hydrogen atom abstraction is a chemical reaction in which a hydrogen free radical is abstracted from a substrate (the activatable molecule) and taken on by the photocatalyst. In such reactions, photoactivation of the photocatalyst results in loss of a hydrogen free radical, thereby activating the photocatalyst to abstract a hydrogen from the activable molecule, returning the photocatalyst to an inactive state. Upon abstraction of the hydrogen, the activatable molecule is converted into an activated molecule.

Example for oxidation-driven uncaging chemistry resulting in liberation of the active from of a molecule include:

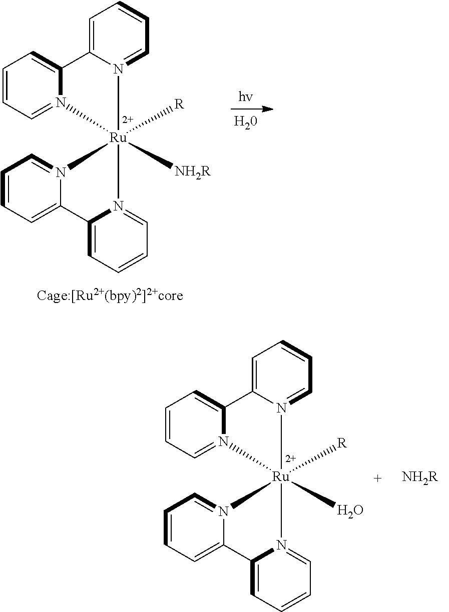

Other oxidation-driven uncaging chemistries find use in embodiments herein.

In some embodiments, conversion of the activatable molecule into the activated molecule comprises catalyzing a redox reaction with the activatable molecule as a substrate for the reaction. In such embodiments, the photocatalyst or photosensitizer absorbs light and is elevated to a redox-active or excited state. The photocatalyst or photosensitizer is then capable of catalyzing a redox reaction to activate the activatable molecule.

In some embodiments, the activatable molecule is a photo-switchable molecule. In some embodiments, a photo-switchable molecule is a molecule that undergoes a reversible conformational change in its structural geometry upon exposure to light energy (e.g., at a specific wavelength), which turn-on its activity (Hüll, K. et. al. In Vivo Photopharmacology Chem. Rev. 2018, 118, 10710-10747; incorporated by reference in its entirety). Examples of activatable photoswitches and the activated molecules they are converted into include:

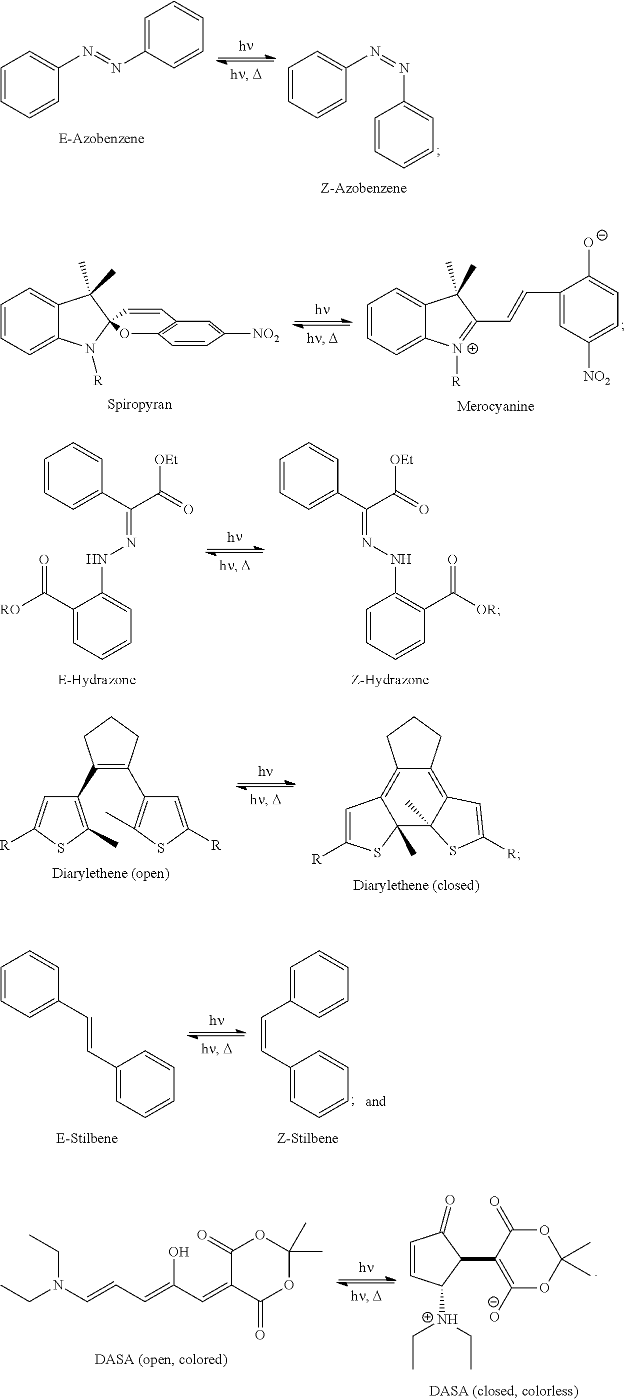

Other activatable/activated photoswitches find use in embodiments herein.

### Localization Elements (HALOTAG)

In some embodiments, two or more components of the systems herein are conjugated (e.g., linked, fused, etc.) to molecular elements that facilitate the localization of the components. In certain embodiments, a bioluminescent protein (or complex) and a photocatalyst are linked together, for example, via molecular localization elements connected to the bioluminescent protein (or complex) and the photocatalyst that bring the bioluminescent protein (or complex) and the photocatalyst into close enough proximity to allow light from a luminophore interacting with the bioluminescent protein (or complex) to activate the photocatalyst.

In some embodiments, the bioluminescent protein or bioluminescent complex is fused to a first molecular entity and the photocatalyst is conjugated to a second molecular entity, wherein interaction of the first and second molecular entities places the bioluminescent protein or bioluminescent complex in sufficient proximity to the photocatalyst such that light emitted by the luminophore upon interaction with the bioluminescent protein or bioluminescent complex activates the photocatalyst. In some embodiments, the first molecular entity is a capture agent (capture protein), and the second molecular entity is a capture element.

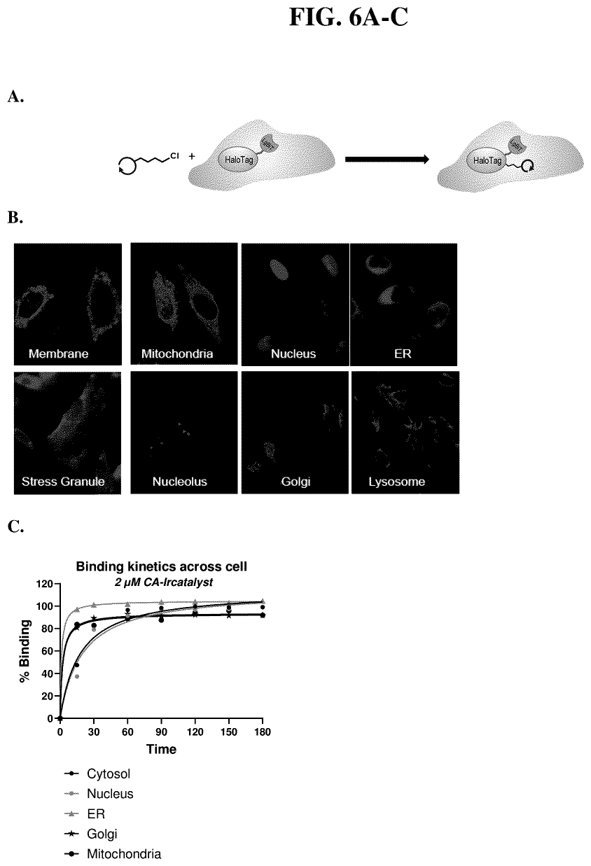

In some embodiments, the bioluminescent protein or bioluminescent complex is fused to a modified dehalogenase capable of forming a covalent bond with its substrate, and wherein the photocatalyst is conjugated to a dehalogenase substrate (See FIG. 6A). In some embodiments, binding of the modified dehalogenase to the dehalogenase substrate places the bioluminescent protein or bioluminescent complex in sufficient proximity to the photocatalyst such that light emitted by the luminophore upon interaction with the bioluminescent protein or bioluminescent complex activates the photocatalyst. In some embodiments, the minimal influence of haloalkane on cell permeability coupled with its highly specific and rapid binding of HaloTag allows for intracellular tethering of a haloalkane conjugate to HALOTAG fused to a component of the system thereby reducing the overall reliance on cellular permeability of components, and allowing localization of the system to a particular cellular compartment (FIG. 6B-C).

In some embodiments, the commercially-available HALOTAG system (Promega Corp.; Madison, WI) is utilized to link or bring together two or more components (e.g., bioluminescent protein or bioluminescent complex and photocatalyst) of the systems and methods described herein. HALOTAG is a 297-residue self-labeling polypeptide (33 kDa) derived from a bacterial hydrolase (dehalogenase) enzyme, which was modified to covalently bind to its ligand, a haloalkane moiety. The HALOTAG ligand can be linked to solid surfaces (e.g., beads) or functional groups (e.g., fluorophores), and the HALOTAG polypeptide can be fused to various proteins of interest, allowing covalent attachment of the protein of interest to the solid surface or functional group.

The HALOTAG polypeptide is a hydrolase with a genetically modified active site, which specifically binds to the haloalkane ligand or chloroalkane linker with an increased rate of ligand binding (Pries et al. The Journal of Biological Chemistry. 270(18): 10405-11: incorporated by reference in its entirety). The reaction that forms the bond between the protein tag and chloroalkane linker is fast and essentially irreversible under physiological conditions (Waugh DS (June 2005). Trends in Biotechnology. 23(6): 316-20; incorporated by reference in its entirety). In the natural hydrolase enzyme, nucleophilic attack of the chloroalkane reactive linker causes displacement of the halogen with an amino acid residue, which results in the formation of a covalent alkyl-enzyme intermediate. This intermediate would then be hydrolyzed by an amino acid residue within the wild-type hydrolase (Chen et al. (February 2005) Current Opinion in Biotechnology. 16(1): 35-40; incorporated by reference in its entirety). This would lead to regeneration of the enzyme following the reaction. However, with HALOTAG, the modified haloalkane dehalogenase, the reaction intermediate cannot proceed through the second reaction because it cannot be hydrolyzed due to a mutation in the enzyme. This causes the intermediate to persist as a stable covalent adduct with which there is no associated back reaction (Marks et al. (August 2006) Nature Methods. 3(8): 591-6; incorporated by reference in its entirety).

HALOTAG fusion proteins can be expressed using standard recombinant protein expression techniques (Adams et al. (May 2002) Journal of the American Chemical Society. 124(21): 6063-76; incorporated by reference in its entirety). Since the HALOTAG polypeptide is a relatively small protein, and the reactions are foreign to mammalian cells, there is no interference by endogenous mammalian metabolic reactions (Naested et al. The Plant Journal. 18(5): 571-6; incorporated by reference in its entirety). Once the fusion protein has been expressed, there is a wide range of potential areas of experimentation including enzymatic assays, cellular imaging, protein arrays, determination of sub-cellular localization, and many additional possibilities (Janssen D B (April 2004). Current Opinion in Chemical Biology. 8(2): 150-9; incorporated by reference in its entirety).

Various HALOTAG ligands, functional groups, fusions, assays, modifications, uses, etc. are described in U.S. Pat. Nos. 8,748,148; 9,593,316; 10,246,690; 8,742,086; 9,873,866; 10,604,745; U.S. Pat. App. 2009/0253131; U.S. Pat. App. 2010/0273186; 20130337539; U.S. Pat. App. 2012/0258470; U.S. Pat. App. 2012/0252048; U.S. Pat. App. 2011/0201024; U.S. 2014/0322794; each of which is incorporated by reference in their entireties.

In some embodiments, a capture protein herein is a circularly permuted modified dehalogenase or split modified dehalogenase.

In some embodiments, a capture protein herein is a modified dehalogenase with an insertion (e.g., bioluminescent protein, component of a bioluminescent complex, circularly permuted bioluminescent protein, circularly permuted component of a bioluminescent complex, extended loop sequence, etc.) within an internal loop.

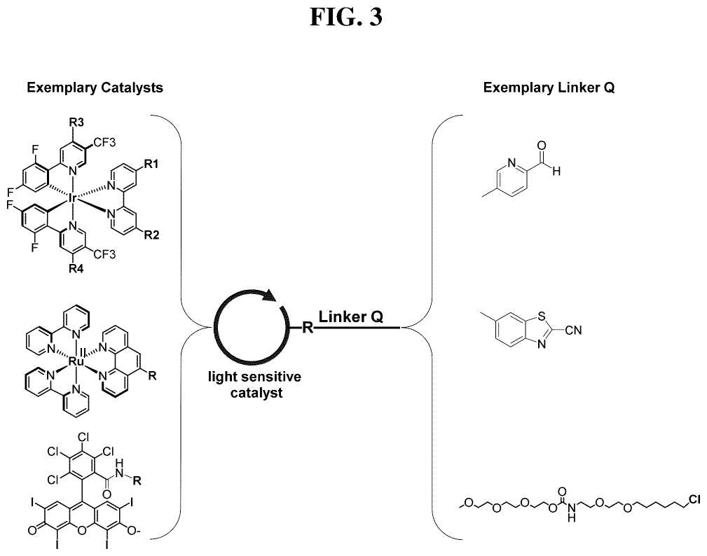

In some embodiments, a first component of the systems herein (e.g., a bioluminescent protein or component of a bioluminescent complex) is fused (e.g., expressed as a fusion) to a modified dehalogenase (e.g., HALOTAG or a variant thereof) or inserted into a surface loop of a modified dehalogenase and a second component of the systems herein (e.g., a photocatalyst) is tethered (e.g., directly or via a linker) to a dehalogenase substrate (e.g., haloalkane). For example, the structure of the photocatalyst tethered to the dehalogenase substrate is P-linker-AX, wherein P is the photocatalyst, wherein A is (CH₂)₂₋₁₂, wherein X is a halogen, and wherein the linker is a linker moiety capable of tethering P to A-X. In some embodiments, the linker is a multiatom straight or branched chain including C, N, S, or O, or a group that comprises one or more rings, e.g., saturated or unsaturated rings, such as one or more aryl rings, heteroaryl rings, or any combination thereof. In some embodiments, the linker comprises a combination of —O(CH₂)₂— —(CH2)O—, —CH₂—, —NHC(O)O—, —OC (O) NH—, NHC(O)—, and —C(O)NH—. In some embodiments, the linker is 5 to 50 (e.g., 5, 10, 15, 20, 25, 30, 35, 40, 45, 50, or ranges therebetween) atoms in length. In some embodiments, the length of the linker for tethering the photocatalyst allows for optimization of proximity and geometry (e.g., for efficient energy transfer). Exemplary linker-A-X groups are depicted in FIG. 3. In some embodiments, a first component of the systems herein (e.g., a bioluminescent protein or component of a bioluminescent complex) is inserted (e.g., expressed as an internal fusion) within a modified dehalogenase (e.g., HALOTAG or a variant thereof) in order to increase proximity or provide a geometry that is favorable of energy transfer to the bound catalyst (See, e.g., U.S. Prov. App. No. 63/338,369; incorporated by reference in its entirety). In some embodiments, the location for insertion within the modified dehalogenase (e.g., HALOTAG or a variant thereof) is selected to provide optimal proximity and geometry for the desired interactions between components, while maintaining the function or activity of the modified dehalogenase (e.g., HALOTAG or a variant thereof) and inserted component.

The scope of embodiments herein is not limited by the types of linkers available. Components and moieties thereof may be linked either directly (e.g., linker consists of a single covalent bond) or linked via a suitable linker. Embodiments are not limited to any particular linker group. A variety of linker groups are contemplated, and suitable linkers could comprise, but are not limited to, alkyl groups, methylene carbon chains, ether, polyether, alkyl amide linker, a peptide linker, a modified peptide linker, a Poly (ethylene glycol) (PEG) linker, a streptavidin-biotin or avidin-biotin linker, polyaminoacids (e.g., polylysine), functionalized PEG, polysaccharides, glycosaminoglycans, dendritic polymers (WO93/06868 and by Tomalia et al. in Angew. Chem. Int. Ed. Engl. 29:138-175 (1990), herein incorporated by reference in their entireties), PEG-chelant polymers (W94/08629, WO94/09056 and WO96/26754, herein incorporated by reference in their entireties), oligonucleotide linker, phospholipid derivatives, alkenyl chains, alkynyl chains, disulfide, or a combination thereof. In some embodiments, the linker is cleavable (e.g., enzymatically (e.g., TEV protease site), chemically, photoinduced, etc.

In some embodiments, a modified dehalogenase (e.g., HALOTAG) and dehalogenase ligand (e.g., haloalkane) are used to tether any two components of the systems and methods described herein (i.e., not limited to tethering of the bioluminescent protein or component of a bioluminescent complex to the photocatalyst). In other embodiments, the bioluminescent protein or component of a bioluminescent complex is tethered to the photocatalyst (or other components described herein) by another mechanism.

In some embodiments, a first component of a system or method herein is linked (e.g., fused) to capture agent (e.g., capture protein) and a second component of the system or method is linked to a capture element. Binding of the capture clement by the capture agent (e.g., capture protein) results in co-localization of the first component and the second component. In some embodiments, the capture agent is a modified dehalogenase, and the capture element is a haloalkane. However, other capture agent/element pairs that may find use in embodiments herein include streptavidin/biotin, antibody (or Ab fragment) and antigen, etc.

In other embodiments, components herein are connected by chemical modification/conjugation, such as by Native chemical ligation, Staudinger ligation, “traceless” Staudinger ligation, amide coupling, methods that employ activated esters, methods to target lysine, tyrosine and cysteine residues, imine bond formation (with and without ortho-boronic acid), boronic acid/diol interactions, disulfide bond formation, copper/copper free azide, diazo, and tetrazine “click” chemistry, UV promoted thiolene conjugation, diazirine photolabeling, Diels-Alder cycloaddition, metathesis reaction, Suzuki cross-coupling, 2-cyanobenzothiazole (CBT) coupling, 2-pyridinecarboxyaldehyde (PCA) coupling etc.

### Target Molecules and Localizing Elements

In some embodiments, an activated molecule interacts with (e.g., binds to) a target molecule (e.g., cellular target, protein, nucleic acid), a chemical moiety, or a cellular compartment.

In some embodiments, the bioluminescent protein or complex is conjugated to a target binding agent, wherein the target binding agent is capable of binding to the target molecule (e.g., protein, nucleic acid, or other biological molecules (e.g., lipid, sugar, etc.)). In some embodiments, the target binding agent is a protein or peptide fused directly or indirectly to the bioluminescent protein or a component of the bioluminescent complex. In some embodiments, the target molecule is a nucleic acid, and the target binding agent is capable of binding specifically or non-specifically to nucleic acids. In some embodiments, the target binding agent is a wildtype or modified Cas protein (e.g., Cas9, dCas9, dCas12, dCas13, etc.) and the target molecule is a nucleic acid that is modified by CRISPR. In some embodiments, systems further comprise a guide RNA (gRNA). In some embodiments, the target molecule is a target peptide or protein, and the target binding agent is capable of binding to the target peptide or protein. In some embodiments, the target binding agent is a small molecule or nucleic acid tethered directly or indirectly to the bioluminescent protein or a component of the bioluminescent complex.

In one set of embodiments, the bioluminescent protein, a component of the bioluminescent complex, the photocatalyst, or the activatable molecule is tethered to a specific ligand, a nucleic acid, or a targeting protein (e.g., Cas9, dCas9, dCas12, dCas13, etc.). Exemplary targeting ligands include small molecule/drug/signaling molecule that bind specifically to the target. In some embodiments, a photocatalyst is tethered to such a small molecule/drug/signaling molecule, thereby allowing localization of the photocatalyst with a protein of interest that is fused to HiBiT or NanoLuc. Other exemplary targeting proteins/ligands include an antibody, antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, oligonucleotide probe, peptide nucleic acid, DARPin, anticalin, nanobody, aptamer, affimer, a purified protein, and analyte binding domain(s) of proteins. Tethering the catalyst to a binding domain that recognize a target protein allows for localization of the catalyst with a protein of interest that is already fused to HiBiT or NanoLuc. In some embodiments, approaches are used to increase proximity with the activatable molecule, for example using a functional moiety that has general affinity to nucleic acids; using a trifunctional molecule comprising a photoreactive moiety; a functional moiety and a recognition moiety that directly bind the target protein; etc.

In this case complementation of HiBiT fused to a protein of interest with LgBiT-HaloTag-catalyst localize the photocatalytic system to a protein of interest. Similarly, LgBiT fused to a protein of interest will localize the protein of interest to a HiBiT-HaloTag-photocatalyst system.

### Systems

In some embodiments, two or more (e.g., 2, 3, 4, or more) of the components of the systems described herein are conjugated together. In some embodiments, one or more pairs of components of the systems described herein are conjugated together. For example, the following pairs of components may be conjugated (e.g., tethered by a linker, genetically fused, etc.): bioluminescent protein and capture protein; photocatalyst and capture ligand; bioluminescent protein and photocatalyst; component of bioluminescent complex and capture protein; component of bioluminescent complex and photocatalyst; bioluminescent protein and target molecule; components of bioluminescent complex and target molecule; bioluminescent protein and target binding agent (e.g., protein, antibody, antibody fragment, antibody-binding agent, nucleic acid, small molecule ligand, etc.); component of bioluminescent complex and target binding agent (e.g., protein, antibody, antibody fragment, antibody-binding agent, nucleic acid, small molecule ligand, etc.); component of bioluminescent complex and photocatalyst; component of bioluminescent complex and capture ligand; activatable molecule and capture ligand; capture protein and target binding agent (e.g., protein, antibody, antibody fragment, antibody-binding agent, nucleic acid, small molecule ligand, etc.); etc.

Components of the systems described herein may be delivered, combined, and/or produced in any suitable manners for a particular application. In embodiments in which the system resides within a cell, components may be expressed within the cell, added exogenously, and allowed to enter the cell (i.e., cell permeable components), or may be delivered to the cell. Delivery of components to the cell may be performed in any suitable delivery vehicle, such as liposomes, micelles, nanoparticles, viruses, etc. In some embodiments, components are tagged to facilitate delivery into cells (e.g., linked to a membrane translocating motif). In some embodiments, components are included to facilitate cellular uptake and/or subsequent endosomal escape. Such additional components may include modified polyethyleneimine polymers and modified poly (amidoamine) dendrimers for use in delivering biomolecules (e.g., delivery of components that cannot passively enter the cell, but cannot be expressed within the cell (e.g., LgBiT/photocatalyst direct conjugates, etc.)) to cells (Sec, U.S. Pub. No. 2020/0399660; incorporated by reference in its entirety).

Exemplary components combinations of systems within the scope herein include:
A dipeptide comprising HIBIT-TRIP9 fused to a protein of interest; fusion of LGTRIP and HALOTAG; photocatalyst tethered to a haloalkyl HALOTAG ligand—HALOTAG binds to the haloalkyl ligand, the high affinity of HIBIT-TRIP9 for LGTRIP forms bioluminescent complex, localize the photocatalytic system to the protein of interest, and bioluminescence triggers the photocatalyst.
A fusion of HIBIT and an antisense oligonucleotide or oligonucleotide probe; LGBIT fused to HALOTAG; photocatalyst tethered to a haloalkyl HALOTAG ligand—HALOTAG binds to the haloalkyl ligand-hybridization of the oligonucleotide to a target nucleic acid sequence localizes the photocatalytic system to a DNA/RNA of interest, and bioluminescence triggers the photocatalyst.
SMBIT fused to an antibody for a target analyte; LGBIT/HALOTAG fused to a general immunoglobulin binding moiety; photocatalyst tethered to a haloalkyl HALOTAG ligand, HALOTAG binds to the haloalkyl ligand—binding of the antibody localizes photocatalytic system to the target analyte, and bioluminescence triggers the photocatalyst.
Fusions of HIBIT and Trip9 to antisense oligonucleotides or oligonucleotide probes that targets the same DNA/RNA of interest; LGTRIP fused to HALOTAG; photocatalyst tethered to a haloalkyl HALOTAG ligand-HALOTAG binds to the haloalkyl ligand, hybridization of the oligonucleotides to a target nucleic acid sequence localizes the photocatalytic system to a DNA/RNA of interest, and bioluminescence triggers the photocatalyst.
HiBiT fused to a protein of interest; fusion of LGBIT and HALOTAG; photocatalyst tethered to a haloalkyl HALOTAG ligand-HALOTAG binds to the haloalkyl ligand, the high affinity of HIBIT for LGBIT forms bioluminescent complex and localize the photocatalytic system to the protein of interest, and bioluminescence triggers the photocatalyst.
dCas9 or dCas12g1 fused to both HaloTag and NanoLuc; photocatalyst tethered to a haloalkyl HALOTAG ligand—HALOTAG binds to the haloalkyl ligand, gRNA targets the photocatalytic system to a DNA/RNA of interest.

### Applications

In some embodiments, the systems and methods herein are utilized to carry-out functional biology analyses reliant on spatiotemporal activation of functional molecules incorporating light-responsive moieties that restrict their activity. A variety of application are made possible by the advantages of the systems and methods herein.

Exemplary applications includes a variety of bioluminescence-triggered photocatalytic activation: 1) Uncaging a photocaged drug for spatiotemporally controlled activation/inhibition of a target protein, activation/inhibition of a population of a target protein localized to a specific cellular compartment, release of a lethal drug and targeted cell death, etc.; 2) Uncaging a photocaged signaling molecule for spatiotemporally controlled activation/inhibition of a signaling pathway; 3) Activation of a photocaged/photoswitched PROTAC for spatiotemporally controlled degradation of a target protein or a population of a target protein localized to a specific compartment; 4) Activation of a photocaged/photoswitched antisense RNA or siRNA for spatiotemporally controlled degradation of RNA or a translation blockade; 5) Uncaging a fluorophore for spatiotemporally controlled detection of RNA and DNA; detection/sorting of HiBiT-edited cells; detecting translocation of a HiBiT-tagged protein to a cellular compartment expressing the LgBiT-HaloTag-catalyst, etc.; and 6) Other applications that make use of the systems, methods, and components herein are within the scope of the technology.

In some embodiments, provided herein are methods of proximity-dependent activation of an activatable molecule within a cell, comprising contacting a cell with a luminophore under conditions in which the luminophore enters the cell, wherein the cell comprises: (a) a fusion of a bioluminescent protein and a capture protein, wherein the bioluminescent protein catalyzes emission of a first wavelength of light from the luminophore upon interaction therewith; (b) a conjugate of (A) a capture ligand and (B) a photocatalyst or photosensitizer, wherein the capture protein forms a covalent bond with the capture ligand upon interaction therewith, and wherein the photocatalyst or photosensitizer is activated by exposure to light of the first wavelength; and (c) an activatable molecule incorporating a light-responsive moiety that restrict its activity, wherein the activatable molecule is converted into an activated molecule when in proximity to the activated photocatalyst or photosensitizer through either cleavage of a photolabile protecting group and liberation of the active molecule, or a transient conformational change that turn on its activity.

In some embodiments, provided herein are methods of inducing a proximity-dependent activation of functional molecules for subsequent interactions with a target molecule, comprising contacting a cell with a luminophore under conditions in which the luminophore enters the cell, wherein the cell comprises: (a) a fusion of a bioluminescent protein and a capture protein, wherein the bioluminescent protein catalyzes emission of a first wavelength of light from the luminophore upon interaction therewith; (b) a conjugate of (A) a capture ligand and (B) a photocatalyst or photosensitizer, wherein the capture protein forms a covalent bond with the capture ligand upon interaction therewith, and wherein the photocatalyst or photosensitizer is activated by exposure to light of the first wavelength; (c) an activatable molecule, wherein the activatable molecule is converted into an activated molecule when in proximity to the activated photocatalyst or photosensitizer; and (d) a target molecule, wherein the activated molecule bind to, interact with the target molecule when in proximity therewith. In some embodiments, the interaction of the activated molecule with the target molecule result in its activation, inhibition, degradation, detection etc.

In some embodiments, provided herein are methods of proximity-dependent activation of an activatable molecule within a cell, comprising: (a) expressing a fusion of a bioluminescent protein and a capture protein within the cell; (b) contacting the cell with a luminophore, under conditions in which the luminophore enters the cell, wherein the bioluminescent protein catalyzes emission of a first wavelength of light from the luminophore upon interaction therewith; (c) contacting the cell with a conjugate of (i) a capture ligand and (ii) a photocatalyst or photosensitizer, under conditions in which the conjugate enters the cell, wherein the capture protein forms a covalent bond with the capture ligand upon interaction therewith, and wherein the photocatalyst or photosensitizer is activated by exposure to light of the first wavelength; and (d) contacting the cell with an activatable molecule, wherein the activatable molecule is converted into an activated molecule when in proximity to the activated photocatalyst or photosensitizer.

In some embodiments, provided herein are methods of inducing a proximity-dependent activation of functional molecules for subsequent interactions with a target molecule, comprising: (a) expressing a fusion of a bioluminescent protein and a capture protein within a cell; (b) contacting the cell with a luminophore, under conditions in which the luminophore enters the cell, wherein the bioluminescent protein catalyzes emission of a first wavelength of light from the luminophore upon interaction therewith; (c) contacting the cell with a conjugate of (i) a capture ligand and (ii) a photocatalyst or photosensitizer, under conditions in which the conjugate enters the cell, wherein the capture protein forms a covalent bond with the capture ligand upon interaction therewith, and wherein the photocatalyst or photosensitizer is activated by exposure to light of the first wavelength; and (d) contacting the cell with an activatable molecule, wherein the activatable molecule is converted into an activated molecule when in proximity to the activated photocatalyst or photosensitizer; wherein the activated molecule interact with the target molecule when in proximity therewith.

In some embodiments, provided herein are methods of proximity-dependent activation of a photocatalyst or photosensitizer within a cell, comprising contacting a cell with a luminophore under conditions in which the luminophore enters the cell, wherein the cell comprises: (a) a fusion of a bioluminescent protein and a capture protein, wherein the bioluminescent protein catalyzes emission of a first wavelength of light from the luminophore upon interaction therewith; (b) a conjugate of (A) a capture ligand and (B) a photocatalyst or photosensitizer, wherein the capture protein forms a covalent bond with the capture ligand upon interaction therewith, and wherein the photocatalyst or photosensitizer is activated by exposure to light of the first wavelength.

In some embodiments, provided herein are methods of proximity-dependent activation of a photocatalyst or photosensitizer within a cell, comprising: (a) expressing a fusion of a bioluminescent protein and a capture protein within the cell; (b) contacting the cell with a luminophore, under conditions in which the luminophore enters the cell, wherein the bioluminescent protein catalyzes emission of a first wavelength of light from the luminophore upon interaction therewith; and (c) contacting the cell with a conjugate of (i) a capture ligand and (ii) a photocatalyst or photosensitizer, under conditions in which the conjugate enters the cell, wherein the capture protein forms a covalent bond with the capture ligand upon interaction therewith, and wherein the photocatalyst or photosensitizer is activated by exposure to light of the first wavelength.

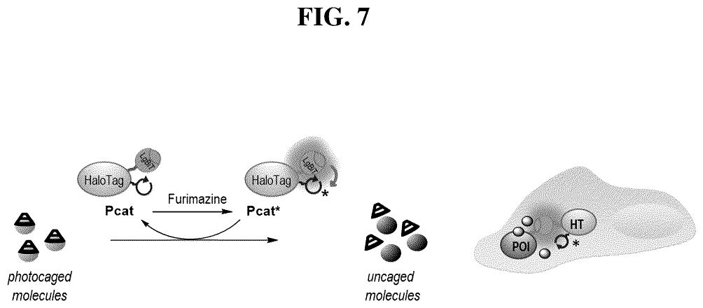

One exemplary general embodiment of the systems and methods herein is depicted in FIG. 7. In this embodiment, a first component of a bioluminescent complex (e.g., LgBiT component of NanoBiT) is conjugated (e.g., fused) to a capture protein (e.g., HALOTAG). In some versions of this embodiment, the first component of the bioluminescent complex and the capture protein are expressed as a fusion within a cell. A photocatalyst is linked to a capture ligand (e.g., comprising a haloalkane). In some embodiments, the photocatalyst linked to the capture ligand is added extracellularly and is capable of entering the cell (e.g., without permeabilizing the cell) and forming a covalent bond with the capture protein. Exposure of the first component of a bioluminescent complex to a second component of the bioluminescent complex (e.g., HiBiT) and a suitable luminophore (e.g., furimazine, fluorofurimazine, etc.) results in formation of an active bioluminescent complex and emission of light. Exposure of the photocatalyst to the light emitted from the bioluminescent complex activates the photocatalyst. The activated photocatalyst subsequently engage in energy transfer events with photocaged molecules within its surrounding vicinity (e.g., molecules incorporating a photolabile protecting groups that restrict their activity) to induce their photocatalytic uncaging via cleavage of protecting groups. The liberated active molecules are then available for interaction with biomacromolecules within their surrounding environment, and/or detection.

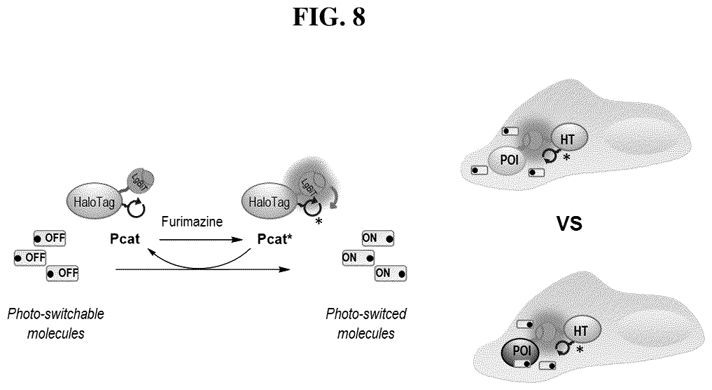

A similar exemplary general embodiment of the systems and methods depicted in FIGS. 7 and 8, except that in FIG. 8 the activatable molecules incorporates a photo switch that restrict their activity. Upon engagement with the activatable molecules, the molecules undergo a reversible conformational change that turn on their activity.

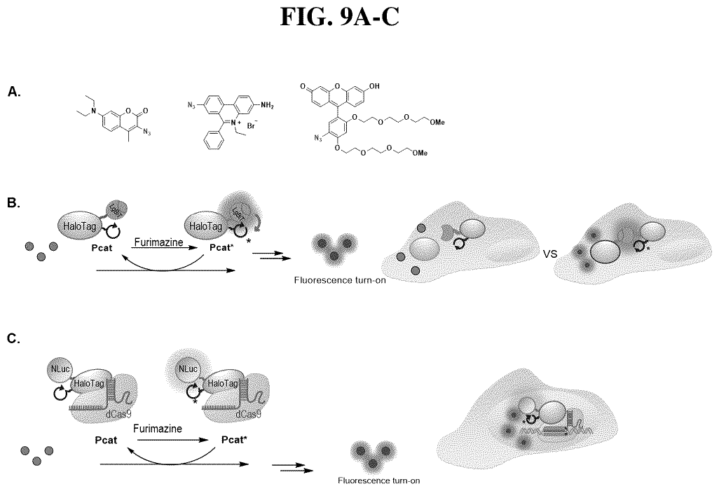

In the embodiment depicted in FIGS. 7 and 8, a bioluminescent complex is utilized as the light source. In other embodiments such as depicted in FIG. 9b, a bioluminescent protein is utilized in place of the bioluminescent complex. The selection of a bioluminescent complex or protein is determined based on the particular application. When applicable, embodiments described for use with one bioluminescent entity here can also find use with other bioluminescent entities herein or as understood in the field.

In some embodiments, the use of complementation to form a bioluminescent complex provides various advantages over other systems (e.g., utilizing a laser or LED as a light source) or systems herein that utilize a bioluminescent protein as the light source. For example, the use of a HiBiT/LgBiT complementation system (or other NanoBiT or NanoTrip-based complementation systems) as the principle light source coupled with a broad toolkit of photocatalyst/activatable molecules offers multiple advantages within living cells or other biological systems. HiBiT is small, minimally perturbing tag, suitable for tagging endogenous target proteins. Tethering LgBiT to a photocatalyst (e.g., via HALOTAG or another capture system) offers a modality agnostic approach to, for example, inducing proximity between the catalyst and protein of interest-tagged with HiBiT, inducing proximity between the catalyst and bioluminescence source (HiBiT/LgBiT), producing greater spatiotemporal resolution through conditional activation (+Furimazine) at a specific site (HiBiT/LgBiT complementation), utilizing chloroalkane chemistry-a convenient approach to tether the catalyst to a HaloTag-LgBiT fusion either biochemically or in cells at a specific compartment expressing the fusion and delivering the photocatalytic system to sites of interest (e.g., intracellularly or extracellularly). The use of a bioluminescent complex (or bioluminescent protein) provides for local delivery of light of an appropriate wavelength (e.g., blue light) for catalyst activation inside intact cells or other complex models. Other embodiments herein utilize a SmBiT/LgBiT complementation system, a HiBiT/Trip9/LgTrip complementation system or another complementation system that requires external complementation (e.g., facilitation) to form a bioluminescent complex. Because for example SmBiT and LgBiT do not form an active bioluminescent complex without facilitation, the use of such components in the systems/methods herein can be used to require an additional localization event (e.g., the binding of an element conjugated (directly or indirectly) to SmBiT to an element conjugated (directly or indirectly) to LgBiT) in order to produce light to activate the photocatalyst.

In other embodiments, the use of a bioluminescent protein provides various advantages over other systems (e.g., utilizing a laser or LED as a light source) or systems herein that utilize a bioluminescent complex as the light source. For example, a bioluminescent protein (e.g., NANOLUC) provides a single-entity light source that can be expressed within cells (e.g., alone or as a fusion with other components of the systems herein). In certain embodiments herein, the enhanced simplicity/efficiency of a single entity light source is preferred over embodiments requiring complementation.

FIGS. 7 and 8 depicts a system that allows for bioluminescence-triggered spatiotemporal photocatalytic activation of activatable molecules in intact cells for interaction with biomacromolecules within their surrounding environment, and/or detection.

Such embodiments may find use in detection, activation, inactivation, and degradation of proximal proteins and nucleic acids as well as probing and altering of biological processes and signaling pathways.

FIG. 9 depicts a system for bioluminescence-triggered spatiotemporal fluorescence turn-on of an azido quench fluorophore. Systems and methods depicted in FIGS. 7 and 8 utilizing complementation between HiBiT genetically fused to a protein of interest and LgBiT genetically fused to HaloTag and tethered to a catalyst are used herein to localize the light source catalyst and site of interest.

Analogous systems depicted in FIG. 9B are useful for targeting the photocatalytic system to a DNA locus of interest. In such embodiments, CRISPR enzymes conjugates (i.e., dCas-NanoLuc-HaloTag-catalyst) are used in combination with gRNA to target a specific DNA/RNA locus. Analogous systems with other protein-or nucleic acid-binding proteins conjugated to the components for the system are within the scope herein. In the CRISPR/dCas system, a guide RNA (gRNA) binds to a specific endogenous target DNA/RNA sequence in a cell. The mutant dCas enzyme binds to the complex of the gRNA and the target sequence. In some embodiments, by linking a bioluminescent protein (NanoLuc) or component of a bioluminescent complex to a component of a CRISPR system, and linking the photocatalyst to the bioluminescent protein (NanoLuc) or component of a bioluminescent complex (e.g., directly or via HaloTag and a HaloTag ligand), the photocatalyst will be activated in proximity of the target DNA/RNA. Although Cas9/dCas9 are the enzyme in CRISPR that are most often used, other Cas and dCas enzymes (e.g., dCas12, dCas13. etc.) can be used in the systems herein to target nucleic acids.

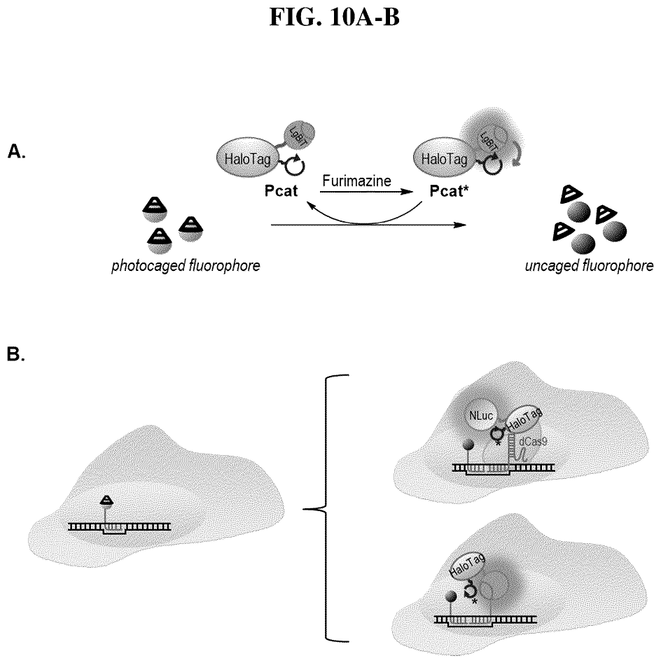

FIG. 10 depict analogous systems for detecting nucleic acids inside cells utilizing a photocaged fluorophore conjugated to an antisense oligo that can further hybridize with a specific nucleic acid sequence. The bioluminescence-triggered photocatalytic system can be localized to a proximal nucleic acid sequence via either a gRNA coupled with a fusion of dCas9-NanoLuc-HaloTag tethered to a catalyst or a complementation between an antisense oligo conjugated to HiBiT and LgBiT genetically fused to HaloTag and tethered to a catalyst. Exposure of the photocatalyst to the light emitted from the bioluminescent enzyme/complex activates the photocatalyst, which subsequently engage in photocatalytic uncaging via cleavage of a protecting group to release the fluorophore.

### EXPERIMENTAL

### Example 1

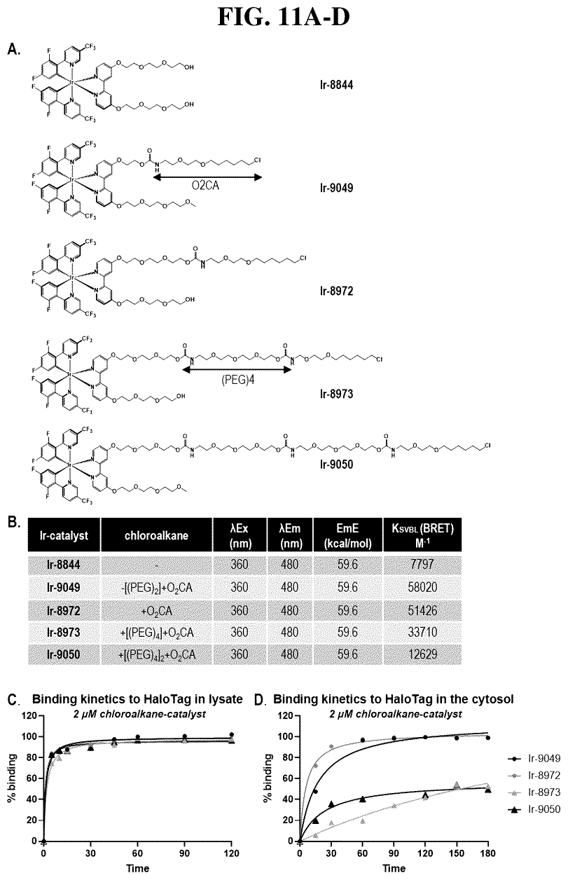

During development of the bioluminescence-triggered photocatalytic activation described herein, experiments were conducted to evaluate the influence of the chloroalkane's length and structure on an iridium catalyst properties including, energy transfer efficiencies (NanoLuc to catalyst), binding kinetic to HaloTag and cellular permeability (FIG. 11). The structures of the modifiable catalyst Ir-8844 and its derivatives further conjugated to chloroalkanes of different lengths are shown in FIG. 11A. The syntheses of these catalysts are included in Example 11.

The influence of the chloroalkanes on the physiochemical properties of the iridium catalysts and their capacity to activated by light emitted from NanoLuc (FIG. 11B) were determined using the following analyses:
a. Excitation and emission profiles: the excitation and emission spectra of 200 μM catalysts in 100% DMSO were monitored on a SPARK multimode plate reader using the following setups: 380 nm excitation for emission scans and either 480 nm, 560 nm, or 600 nm emission for excitation scans.
b. Emission energies (EmE) were calculated from λ_{Em} using the Planck's equation

$$E ( J ) = ( h \times c ) / λ$$

where h (Planck's constant)=6.625×10⁻³⁴ J×sec; c(speed of light)=3×10⁸ m/sec and λ(m) is λ_{Em} Energies in Joules were converted to cal/mol using the conversion of 1 J=0.239 Cal:

$$E ( \frac{cal}{mol} ) = J \times 0.239 \times Avogadro number$$

where Avogadro number=6.02214×10²³
c. Energy transfer from NanoLuc to the catalysts was determined using a modified Stern-Volmer quenching relationship analysis monitoring the capacity of increasing concentrations of catalyst (0-125 μM) to quench the bioluminescence emission of 0.6nM NanoLuc following the addition of 20 μM NanoGlo® Live Cell substrate in TBS+0.01% BSA (Promega Corporation, Cat. No. N205).

$$\frac{{B}^{0} - B}{B} = {k}_{svBL} [ Q ]$$

where the quenching rate k_{SVBL} corresponds to the efficiency of energy transfer and can be determined as the slop for a plot of

$$\frac{{B}^{0} - B}{B}$$

against [Q].

These analyses revealed that the chloroalkane increased the efficiency of energy transfer from NanoLuc to the catalyst (2-7-fold) in a manner that was inversely correlated to the chloroalkane length. Since the chloroalkane had no impact on catalysts' emission energy (EmE), these results indicate that the chloroalkane increased the capacity of the catalyst to absorb light in a manner that was inversely correlated with the chloroalkane length.

The chloroalkane provides the means to induce proximity between the catalyst and bioluminescence light source through covalent binding of a chloroalkane-catalyst conjugate to HaloTag genetically fused to the light source. The influence of the chloroalkane length and structure on binding kinetics to HaloTag (FIG. 11C) was evaluated by treating lysate prepared from cells expressing a HaloTag fusion protein with chloroalkane-catalyst conjugates at a final concentration of 2 μM. After 0-120 minutes incubation time points, a fraction of each reaction (each containing a different chloroalkane-catalyst conjugate) was removed and treated with HaloTag TMR-fluorescent ligand (Promega) at a final concentration of 5 μM. This allowed binding of the fluorescent ligand to any HaloTag fusion protein that remained unbound. The time point fractions were resolved on SDS-PAGE and scanned on a Typhoon fluorescent imager (GE healthcare). Bands were quantified using ImageQuant (GE healthcare), and binding kinetics were determined as the percent binding with time relative to time zero when no chloroalkane-catalyst conjugate was added. All chloroalkane-catalyst conjugates, regardless of the chloroalkane length, exhibited similar binding kinetic to HaloTag indicating that the length of the chloroalkane had very minimal impact on binding kinetic.

The influence of the chloroalkane length and structure on cellular permeability of catalyst conjugates was further evaluated through their binding kinetics to HaloTag inside cells (FIG. 11D). To this end, cells expressing a HaloTag fusion protein were treated with chloroalkane-catalyst conjugates at a final concentration of 2 μM for 0-180 minutes before being treated for additional 15 minutes with HaloTag TMR-fluorescent ligand at a final concentration of 5 μM. This allowed binding of the fluorescent ligand to any HaloTag fusion protein that remained unbound. Cells were then collected, lysed with detergent lysis buffer, and time points analyzed as described above. This analysis revealed that shorter chloroalkanes had minimal impact on cellular permeability allowing for rapid binding kinetics to HaloTag inside cells.

### Example 2

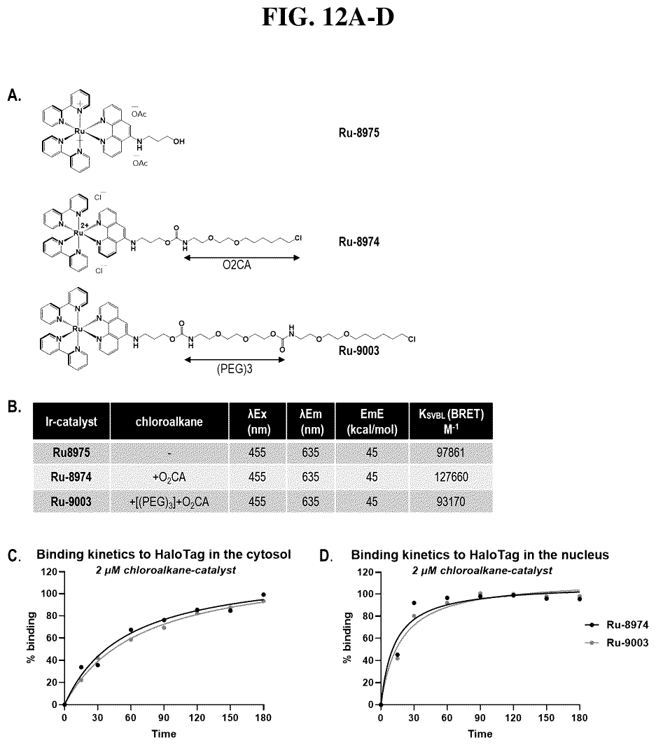

During development of the bioluminescence-triggered photocatalytic activation described herein, experiments were conducted to evaluate the influence of the chloroalkane's length and structure on a ruthenium catalyst properties including, energy transfer efficiencies (NanoLuc to catalyst), binding kinetic to HaloTag and cellular permeability (FIG. 12). The structures of the modifiable catalyst Ru-8975 and its derivatives further conjugated to chloroalkanes of different lengths are shown in FIG. 12A. The syntheses of these catalysts are included in Example 11.

The influence of the chloroalkanes on the physiochemical properties of the ruthenium catalysts and their capacity to activated by light emitted from NanoLuc (FIG. 11B) were determined using the analyses described in Example 1. These analyses revealed that the red shifted emission of the ruthenium catalysts results with a lower Emission Energy (EmE) compared to ruthenium catalysts. At the same time, the red shifted excitation and greater overlap with NanoLuc emission resulted with a significantly higher energy transfer efficiency from NanoLuc to the catalyst and subsequently a significantly smaller impact (1-1.3-fold) of the chloroalkane on the efficiency of energy transfer.

The influence of the chloroalkane length and structure on cellular permeability of catalyst conjugates was further evaluated through their binding kinetics to HaloTag in the cytosol and nucleus of cells as described in example 1 (FIG. 11C and D). This analysis reveal the two relatively short chloroalkanes had similar influence on catalyst binding kinetics and cellular permeability.

### Example 3

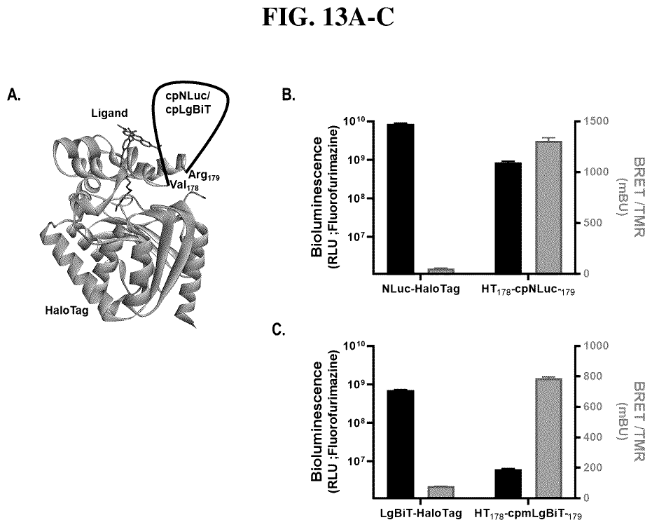

This example describes further optimization of the bioluminescent photocatalytic complex comprising a bioluminescent energy donor, chloroalkane-catalyst conjugate, and HaloTag, which offers the means to induce proximity between the two (FIG. 13). To increase proximity, a chimeric structure was engineered comprising a circularly permuted NanoLuc (e.g., cpNLuc at residues 67/68) or a circularly permuted LgBiT mutant incorporating 4 mutations from LgTrip E4D, Q42M, M106K, T144D (e.g., cpmLgBiT at residues 67/68) that is inserted into a HaloTag's surface loop (between residues 178-179), which is proximal to the ligand interaction site (i.e., HT₁₇₈-cpNLuc-₁₇₉ and HT₁₇₈-cpmLgBIT-₁₇₉) (FIG. 13A). First, NanoLuc-HaloTag and the chimera HT₁₇₈-cpNLuc-₁₇₉ were compared for efficiency of bioluminescence resonance energy transfer (BRET) to a bound HaloTag TMR-fluorescent ligand. To this end, NanoLuc-HaloTag and HT₁₇₈-cpNLuc-₁₇₉ unconjugated or conjugated to a HaloTag TMR-fluorescent ligand were diluted in TBS+0.01% BSA to a final concentration of 6.6 nM and then treated with 10× fluorofurimazine at a final concentration of 20 μM. Following a 3 minute incubation, raw luminescence (Total RLU) or filtered luminesces for donor (e.g., 450 nm/8 nm BP) and acceptor (600 nm LP) emissions, respectively were measured on a GloMax® Discover plate reader (Promega). BRET ratios were further calculated for each sample by dividing the acceptor emission value by its donor emission value. Although HT₁₇₈-cpNLuc-₁₇₉ was 10-fold dimmer, it provided 24-fold greater BRET efficiency (FIG. 13B) indicating that the chimeric structure was able to induce greater proximity between NanoLuc's substrate binding site and the bound fluorescent ligand or adopt a conformation favorable for energy transfer between the two or both. Similar analysis was performed for LgBiT-HaloTag and the chimera HT₁₇₈-cpmLgBiT-₁₇₉. To this end, LgBiT-HaloTag and the chimera HT₁₇₈-cpmLgBiT-₁₇₉ unconjugated or conjugated to a HaloTag TMR-fluorescent ligand were first diluted in TBS+0.01% BSA to a final concentration of 13 nM and allowed to complement with equal volume of 130 nM of HiBiT peptide for 30 min before being treated with 10× fluorofurimazine at a final concentration of 20 μM. Following a 3 minute incubation, raw luminescence (Total RLU) or BRET were measured as described above. Resulted indicated that even 100-fold dimmer HT₁₇₈-cpmLgBiT-₁₇₉. provided 10-fold greater BRET efficiency (FIG. 13B) indicating that the chimeric structure was able to induce greater proximity between HiBiT/LgBiT complex's substrate binding site and the bound fluorescent ligand or adopt a conformation favorable for energy transfer between the two or both.

### Example 4

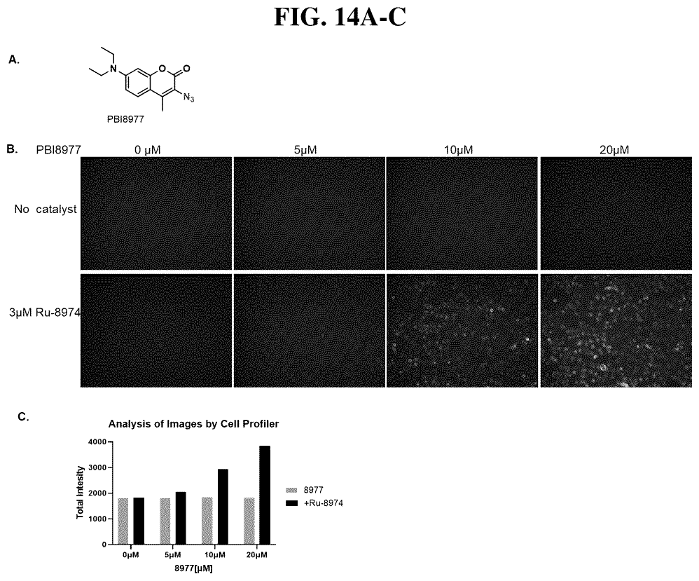

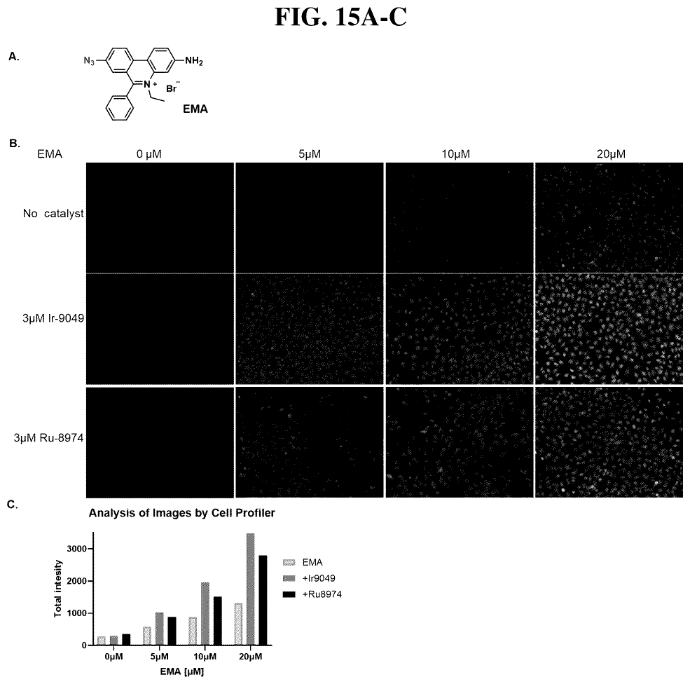

This example demonstrates the feasibility a bioluminescent photocatalytic complex assembled inside cells to drive uncaging of azido-quenched fluorophores (FIGS. 14 and 15). Structure of exemplary azido quenched fluorophores and a cartoon depicting bioluminescent-triggered fluorescence turn-on are included in FIG. 9. Briefly, upon absorbance of light, the excited catalyst engages in energy transfer events with the azido-quenched fluorophore to generate a nitrene through elimination of an N₂ group, which restore the fluorophore's fluorescence. HeLa cells were transfected with a DNA construct encoding HT₁₇₈-cpNLuc-₁₇₉ that was diluted 50-fold into promoterless carrier DNA, plated in flasks at 2×10⁵ cell/mL, and incubated 16-18 hours at 37° C., 5% CO₂. The next day, cells were collated, replated in 24-well plates at 2×10⁵ cell/mL, and incubated overnight at 37° C., 5% CO₂. The next day, plates were treated for 90 minutes with either Ir-9049 catalyst or Ru-8974 catalyst or chloroalkane-biotin (control) at final concentrations of 3 μM to allow assembly of bioluminescent photocatalytic complexes inside cells. Cells were then washed twice, 15 minutes each, in HBSS buffer to remove excess unreacted catalyst or chloroalkane-biotin. After the last wash, the HBSS wash was replaced with Opti-MEM media supplemented with 2% serum and 2-20 μM azido-quenched Coumarin (PBI-8977, synthesis included in Example 11 or 2-20 μM azido-quenched Ethidium Bromide (Ethidium Monoazide Bromide (EMA); Thermo Fisher). Following a 30-minutes incubation, cells were treated with 20 μM fluorofurimazine for 45 minutes. Cells were washed twice before imaging on a BZ-X800 Analyzer (Keyence) and image analysis using cell profiler software. Images of cells treated with either PBI-8977 (FIG. 14B-C) or EMA (FIG. 15B-C) revealed pronounced catalyst dependent increase in fluorescence demonstrating the capacity of the bioluminescent photocatalytic complex to drive uncaging of azido quenched fluorophore inside cells.

### Example 5

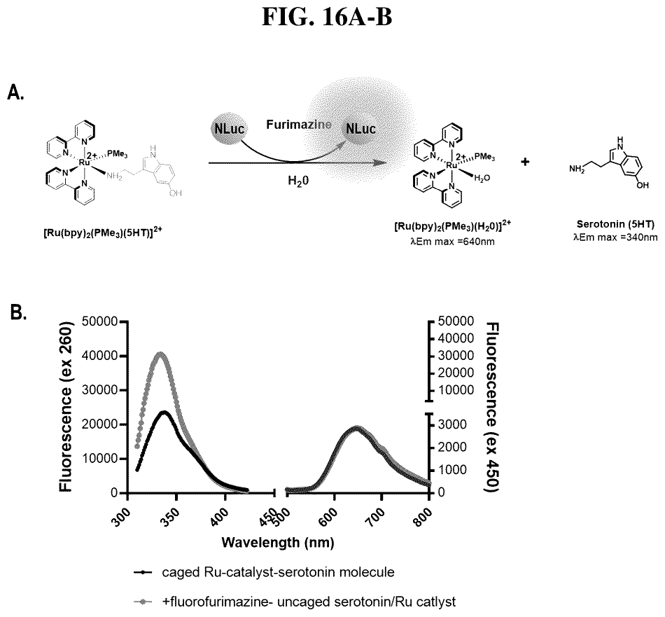

This example demonstrates the feasibility a bioluminescent-triggered release of a signaling molecule, from a caging transition metal complex [Ru²⁺(bpy)₂]² within a biochemical setting. As depicted in FIG. 16A, the amino-signaling molecule serotonin is caged through a coordination reaction with the [Ru²⁺(bpy)₂]₂ core. Upon absorbance of light, the excited ruthenium catalyst induces an oxidation-driven release of serotonin while a water molecule occupies the vacant coordination site.

Here, 6 nM NanoLuc (Nluc) enzyme and 5 μM fluorofurimazine, in the presence and absences of 200 μM RuBi5 (Abcam), were incubated in a white plate for 40 minutes. Reactions were then transferred to a black plate, and 200 μM RuBi5 (caged) added to the control wells. Fluorescence emission scans using 260 nm and 450 nm excitation on the SPARK multimode plate reader (Tecan) revealed bioluminescence-dependent increase in the 340 nm peak emission for serotonin. These results demonstrated that bioluminescence triggered the oxidation driven release of a caged molecule from an organometallic caging moiety.

### Example 6

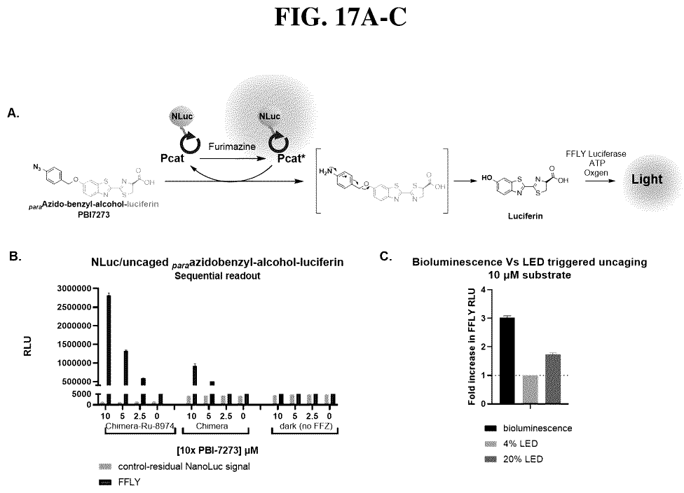

This example demonstrates the feasibility for bioluminescent-triggered uncaging upon Ru-catalyzed p-azidobenzyl reduction in a biochemical setting (FIG. 17). As depicted in FIG. 17A, bioluminescence-triggered ruthenium-catalyzed reduction of p-azidobenzyl-caged luciferin to liberate luciferin, which is then available as substrate for Firefly (FFLY) luciferase. Synthesis of p-azidobenzyl-caged luciferin is provided in Example 11. Briefly, reactions comprising 60 nM HT₁₇₈-cpNLuc-₁₇₉-tethtered and untethered to Ru-8974 in TBS +0.01% BSA+1 mM NaAscorbate and 0-10 μM p-azidobenzyl-luciferin were incubated for 45 minutes in 96 well plates with and without 20 μM fluorofurimazine. Residual NanoLuc bioluminescence signal was quenched by treatment with 10 mM competitive extracellular NanoLuc inhibitor before subsequent 5-fold dilution into FFLY detection plate comprising FFLY luciferase, ATP, and Bright-Glo buffer. Bioluminescence was measured before and after dilution into the FFLY detection plate. Bioluminescence and Ru-dependent increase in FFLY dependent bioluminescence demonstrates the capacity for bioluminescence to drive photocatalytic uncaging of p-azidobenzyl-caged molecules. Furthermore, the higher efficiency for bioluminescence-driven uncaging compared to LED further demonstrates the advantage for proximity-driven catalyst activation via BRET offering highly efficient light delivery to the site of interest.

### Example 7

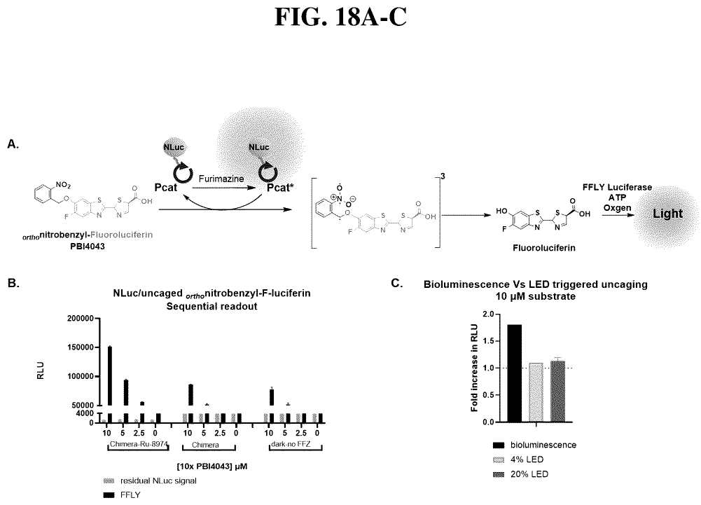

This example demonstrates the feasibility for bioluminescent-triggered uncaging excitation of o-nitrobenzyl and subsequent photolysis a biochemical setting (FIG. 18). As depicted in FIG. 18A, bioluminescence-triggered ruthenium-catalyzed excitation of o-nitrobenzyl-caged F-luciferin to liberate F-luciferin, which is then available as a substrate for FFLY luciferase. Synthesis of o-nitrobenzyl-caged F-luciferin is provided in Example 11. Briefly, reactions comprising 60 nM HT₁₇₈-cpNLuc-₁₇₉-tethtered and untethered to Ru-8974 in TBS+0.01% BSA+1 mM NaAscorbate and 0-10 μM of o-nitrobenzyl-F-luciferin were incubated for 45 minutes in 96 well plates with and without 20 μM fluorofurimazine. Residual NanoLuc bioluminescence signal was quenched by treatment with 10 mM competitive extracellular NanoLuc inhibitor before subsequent 5-fold dilution into FFLY detection plate comprising FFLY luciferase, ATP, and Bright-Glo buffer. Bioluminescence was measured before and after dilution into the FFLY detection plate. Bioluminescence and Ru-dependent increase in FFLY dependent bioluminescence demonstrates the capacity for bioluminescence to drive photocatalytic uncaging of o-nitrobenzyl-caged molecules. Furthermore, the higher efficiency for bioluminescence-driven uncaging compared to LED further demonstrates the advantage for proximity-driven catalyst activation via BRET offering highly efficient light delivery to the site of interest.

### Example 8

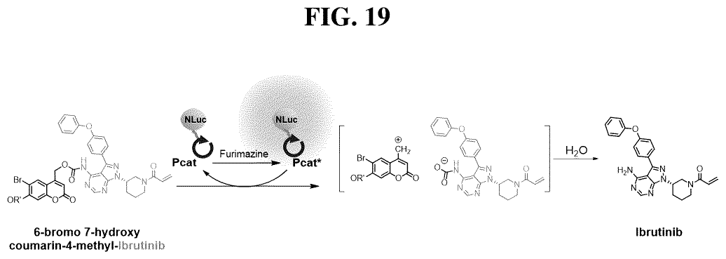

The example depicted in FIG. 19 portrays bioluminescence-triggered photocatalytic uncaging upon excitation of a caging coumarin moiety. Upon treatment of a bioluminescent complex comprising HT₁₇₈-cpNLuc-₁₇₉-tethtered to a catalyst with fluorofurimazine, the excited catalyst engages in energy transfer events with proximal coumarin-4-methyl-caged Ibrutinib. The excited coumarin-4-methyl further undergo photolysis to liberate ibrutinib.

### Example 9

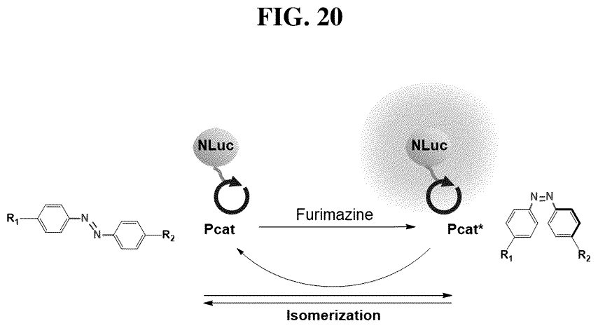

The example depicted in FIG. 20 portrays bioluminescence-triggered transient conformational change to turn-on the reactivity of an effector molecule with a target biomacromolecule. Upon treatment of a bioluminescent complex comprising HT₁₇₈-cpNLuc-₁₇₉-tethtered to a catalyst with fluorofurimazine, the excited catalyst engages in photosensitization of and azobenzene moiety, which upon excitation possess a significantly lower energetic barrier for rotation around the N═N double bond allowing for subsequent conformational change that impact either the distance and/or orientation of R1 and R2 with respect to each other.

### Example 10

This example describes the synthesis of the catalysts and activatable molecules described herein.

| Catalyst | Structure | MS | Ex/nm | Em/nm |
|---|---|---|---|---|
| Ir-8844 | 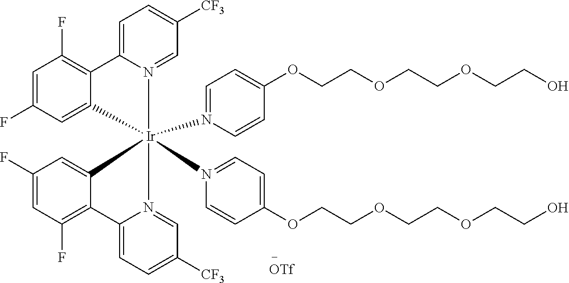 | [M]+ 1161.12 | 380 | 480 |
| Ir-9049 | 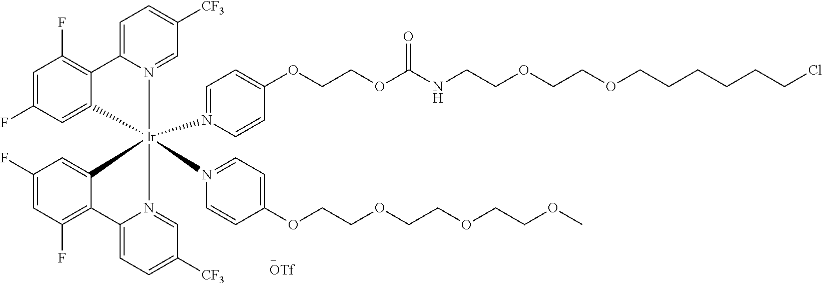 | [M]+ 1336.72 | 380 | 530 |
| Ir-8972 | 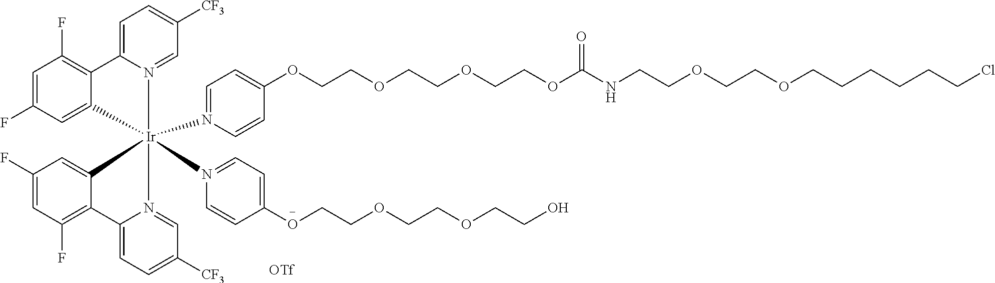 | [M]+ 1410.36 | 380 | 530 |
| Ir-8973 | 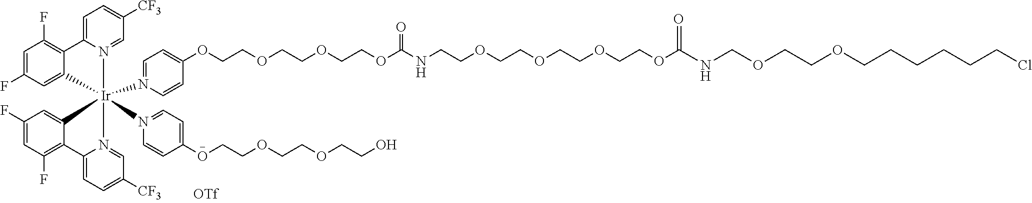 | [M]+ 1410.36 | 380 | 530 |
| Ir-9050 | 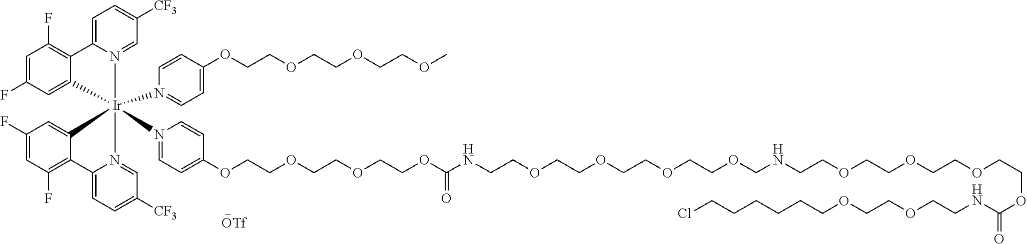 | [M]+ 1863.30 | 380 | 530 |
| Ru-8975 | 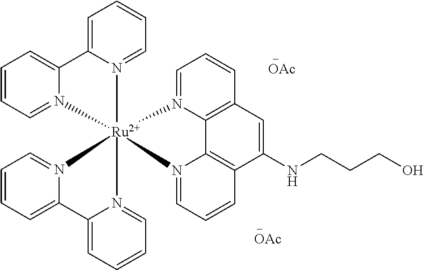 | [M]²⁺/2 333.58 | 450 | 620 |
| Ru-8974 | 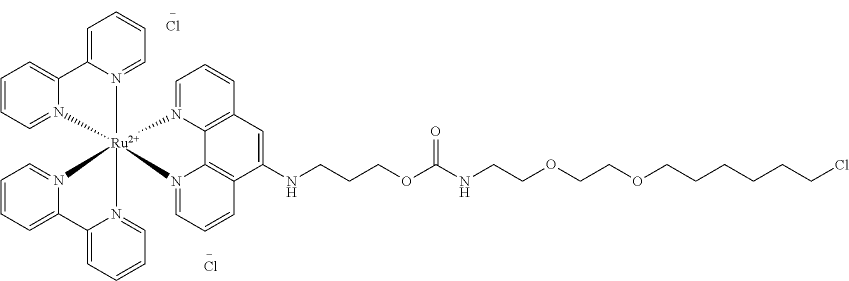 | [M]²⁺/2 458.14 | 450 | 620 |
| Ru-9003 | 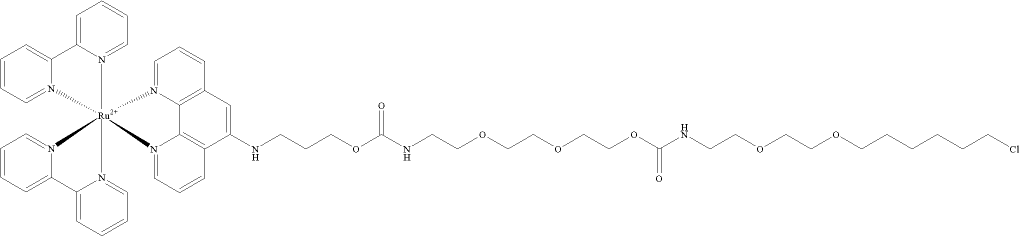 | [M]²⁺/2 545.84 | 450 | 620 |

### Syntheses of Ir catalysts:

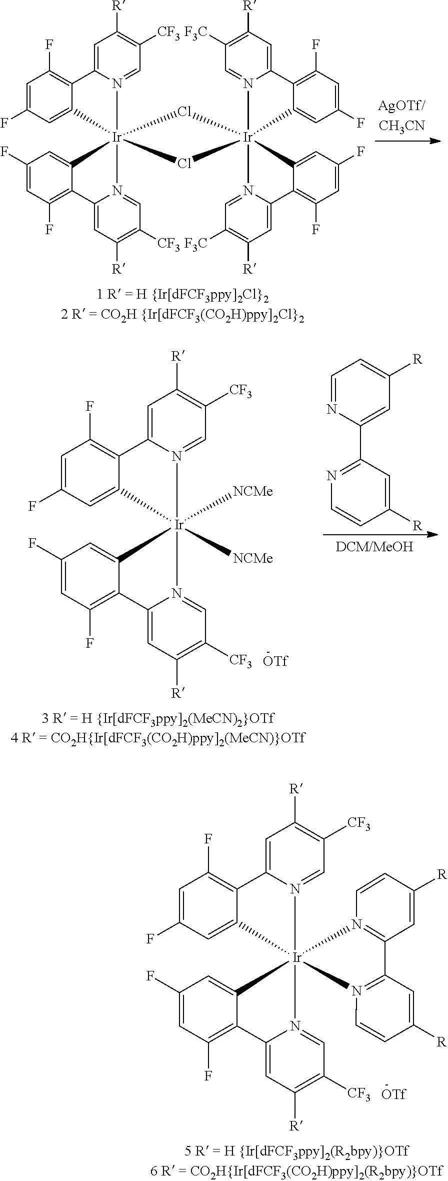

{Ir[dFCF₃ppy]₂Cl}₂ is commercially available from Strem: www.strem.com/catalog/v/77-0468/31/iridium_870987-64-7, and {Ir[dFCF₃(CO₂H)ppy]₂Cl}₂ was synthesized following literature reported procedures: Science 367, 1091-1097 (2020).

GP1: The bi-Ir-Cl complex (0.1 mmol, 1.0 equiv) was combined with AgOTf (53 mg, 0.2 mmol, 2.0 equiv) in CH₃CN (5 mL). This mixture was stirred at RT overnight in the dark. The resulting suspension was then filtered through celite and concentrated. The residue was redissolved DCM/MeOH (1/1, 10 mL), filtered through celite, and concentrated to yield the intermediate 3 or 4 as yellow film that was used without further purification. To a solution of Intermediate 3 or 4 (0.1 mmol, 1.0 equiv) in DCM/MeOH (1/1, 2 mL), bpy was added to the reactants (0.12 mmol, 1.2 equiv). The reaction mixture was then stirred at RT for 16 h. LC-MS indicated full conversion of intermediates 3 or 4. The solution was evaporated onto celite and purified by silica gel chromatography.

Ir-8844: ¹H NMR (400 MHZ, Methylene Chloride-d₂) δ8.48 (d, J=8.7 Hz, 2H), 8.37 (d, J=2.5 Hz, 2H), 8.07 (d, J=8.9 Hz, 2H), 7.70 (d, J=6.2 Hz, 4H), 7.12-6.98 (m, 2H), 6.73-6.57 (m, 2H), 5.73 (dd, J=8.1, 2.2 Hz, 2H), 4.65-4.43 (m, 4H), 3.94 (d, J=4.5 Hz, 4H), 3.79-3.42 (m, 16H). LRMS [M]⁺1161.1.

### Synthesis of bpy-1

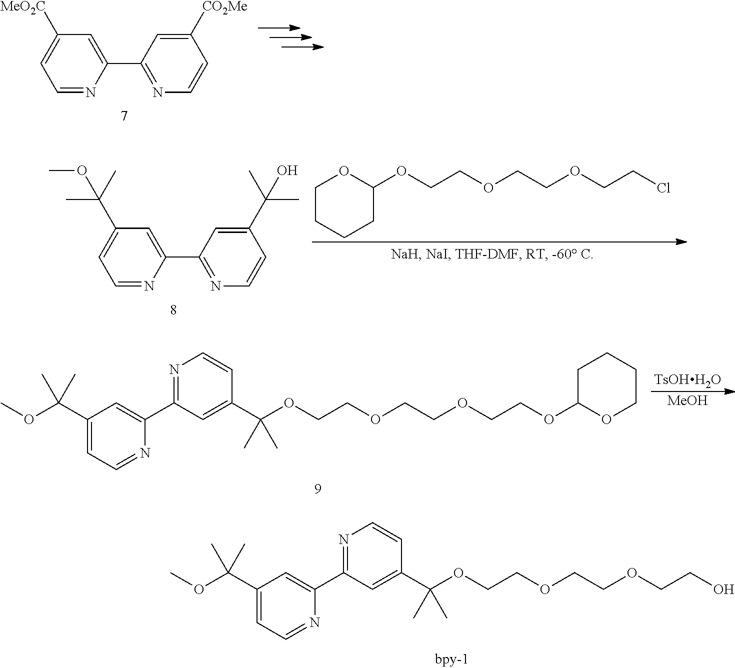

Intermediate 8 was synthesized from commercially available starting material 7 following literature procedure: Science 367, 1091-1097 (2020).

Intermediate 9: To a solution of intermediate 8 (200 mg, 0.7 mmol, 1.0 equiv) in THF (7 mL), NaH (60 wt %, 56 mg, 1.4 mmol, 2.0 equiv) was added. The mixture was stirred at RT for 30 min. To the suspension, NaI (11 mg, 0.07 mmol, 0.1 equiv) and 2-(2-(2-(2-chloroethoxy) ethoxy) ethoxy) tetrahydro-2H-pyran (350 mg, 1.4 mmol, 2.0 equiv) in DMF (3 mL) was added dropwise over 10 min. The mixture was then heated at 60° C. for 48 h. The reaction was cooled down and quenched by addition of saturated aq. NH₄Cl (10 mL). The quenched reaction was then concentrated in vacuo to remove organic solvents and extracted with EtOAc (20×3 mL). The combined organic layers were washed with H₂O (50 mL), brine (50 mL), dried over Na₂SO₄ and concentrated to afford the crude, which was used in the next step without further purification.

bpy-1: Intermediate 9 (50 mg, 0.1 mmol, 1.0 equiv) and TsOH·H₂O (19 mg, 0.1 mmol, 1.0 equiv) were dissolved in MeOH (4 mL). The solution was stirred at RT for 2 h. LC-MS indicated full conversion. The reaction was concentrated onto celite, and the desired product was isolated using silica gel chromatography. 1H NMR (400 MHZ, Chloroform-d) δ8.64 (d, J=5.1 Hz, 2H), 8.38 (d, J=5.5 Hz, 2H), 7.41 (ddd, J=19.4, 5.0, 2.2 Hz, 2H), 3.83-3.53 (m, 10H), 3.38 (t, J=5.2 Hz, 2H), 3.13 (s, 3H), 1.59 (s, 6H), 1.56 (s, 6H). LRMS [M+H]⁺419.5.

### Synthesis of bpy-2

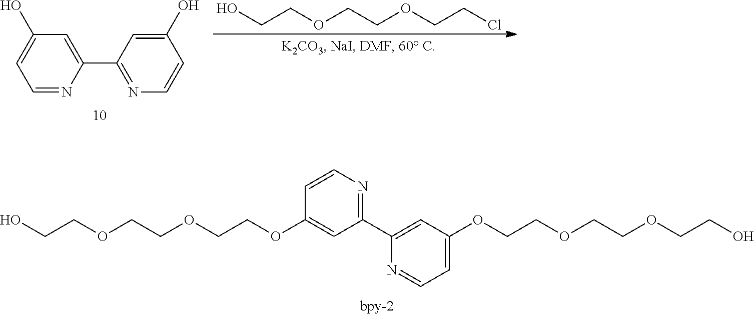 [SMILES: Oc1ccnc(-c2cc(O)ccn2)c1 ; OCCOCCOCCCl ; OCCOCCOCCOc1ccnc(-c2cc(OCCOCCOCCO)ccn2)c1]

bpy-2: To a suspension of bpy 10 (376 mg, 2.0 mmol, 1.0 equiv) and K₂CO₃ (830 mg, 6.0 mmol, 3.0 equiv) in DMF (5 mL), 2-(2-(2-chloroethoxy) ethoxy) ethan-1-ol (1.0 g, 6.0 mmol, 3.0 equiv) was added dropwise over 5 min. The mixture was heated at 60° C. for 20 h and cooled down to RT, diluted with EtOAc (100 mL), and filtrated over celite. The filtrate was concentrated in vacuo to afford the crude. The desired product, bpy-2, was isolated by silica gel chromatography. ¹H NMR (400 MHz, Methanol-d₄) δ8.46 (dd, J=5.8, 1.8 Hz, 2H), 7.86 (d, J=2.2 Hz, 2H), 7.05 (dt, J=5.3, 2.3 Hz, 2H), 4.34 (t, J=4.2 Hz, 4H), 3.92 (p, J=2.2 Hz, 4H), 3.81-3.47 (m, 16H). LRMS [M+H]⁺453.3.

### Synthesis of Ir-8972

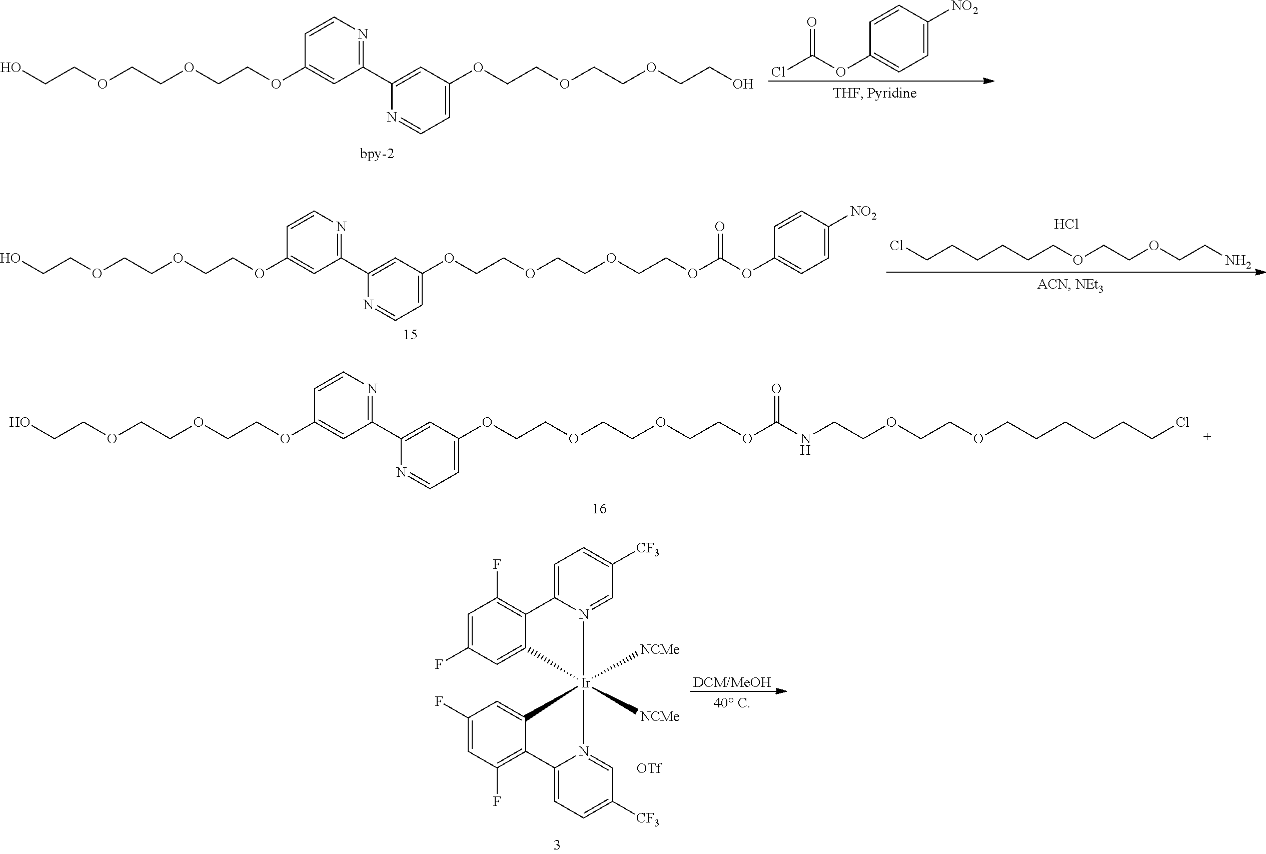

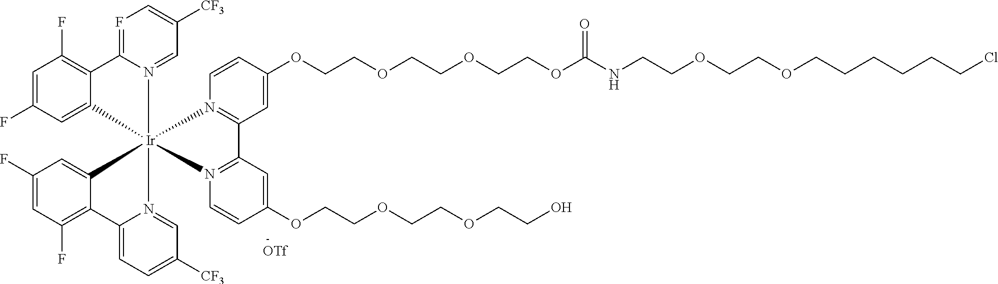

Intermediate 16: To a solution of bpy-2 (25 mg, 0.06 mmol, 1.0 equiv) in THF (4 mL), pyridine (0.5 mL) and p-nitrophenyl chloroformate (15 mg, 0.07 mmol, 1.2 equiv) was added. The solution was stirred at RT overnight. The reaction was diluted with DCM (10 mL), filtered over Celite, and the filtrate was concentrated in vacuo to afford the crude, intermediate 15, which was used in the next step without further purification.

To a solution of the crude 15 (54 mg, 87 μmol, 1.0 equiv) in ACN (2 mL), the chloroalkane amine reactive agent (15 mg, 92 μmol, 1.1 equiv) and NEt₃ (0.2 mL) was added. The solution was stirred at RT overnight and concentrated onto celite. The desired product was isolated using silica gel chromatography. ¹H NMR (400 MHZ, Methanol-d₄) δ8.47 (d, J=5.8 Hz, 2H), 7.88 (d, J=2.6 Hz, 2H), 7.07 (dd, J=5.6, 2.6 Hz, 2H), 4.35 (t, J=4.4 Hz, 4H), 4.17 (d, J=5.0 Hz, 2H), 3.93 (q, J=3.3 Hz, 4H), 3.77-3.64 (m, 12H), 3.61-3.43 (m, 12H), 3.30-3.28 (m, 2H), 1.76 (t, J=7.1 Hz, 2H), 1.66-1.30 (m, 6H). LRMS [M+H]⁺702.3.

Ir-8972: Intermediate 3 (10.6 mg, 11 μmol, 1.0 equiv) and intermediate 16 (8 mg, 12 μmol, 1.1 equiv) was dissolved in DCM/MeOH (1/1, 2 mL). The solution was stirred at RT overnight. The desired product was isolated using prep-HPLC using 0.1% TFA in H₂O and ACN as mobile phases. 1H NMR (400 MHZ, Methanol-d₄) δ8.56 (d, J=9.0 Hz, 2H), 8.42-8.24 (m, 4H), 7.82 (d, J=6.3 Hz, 4H), 7.25 (d, J=6.8 Hz, 2H), 6.79 (t, J=10.9 Hz, 2H), 5.77 (d, J=8.1 Hz, 2H), 4.45 (d, J=4.7 Hz, 4H), 4.07 (s, 2H), 3.90 (d, J=4.7 Hz, 4H), 3.77-3.40 (m, 24H), 3.30-3.23 (d, J=6.1 Hz, 2H), 1.78-1.66 (m, 2H), 1.56 (t, J=7.2 Hz, 2H), 1.43-1.35 (m, 4H). LRMS [M]⁺1410.4.

### Synthesis of Ir-8973

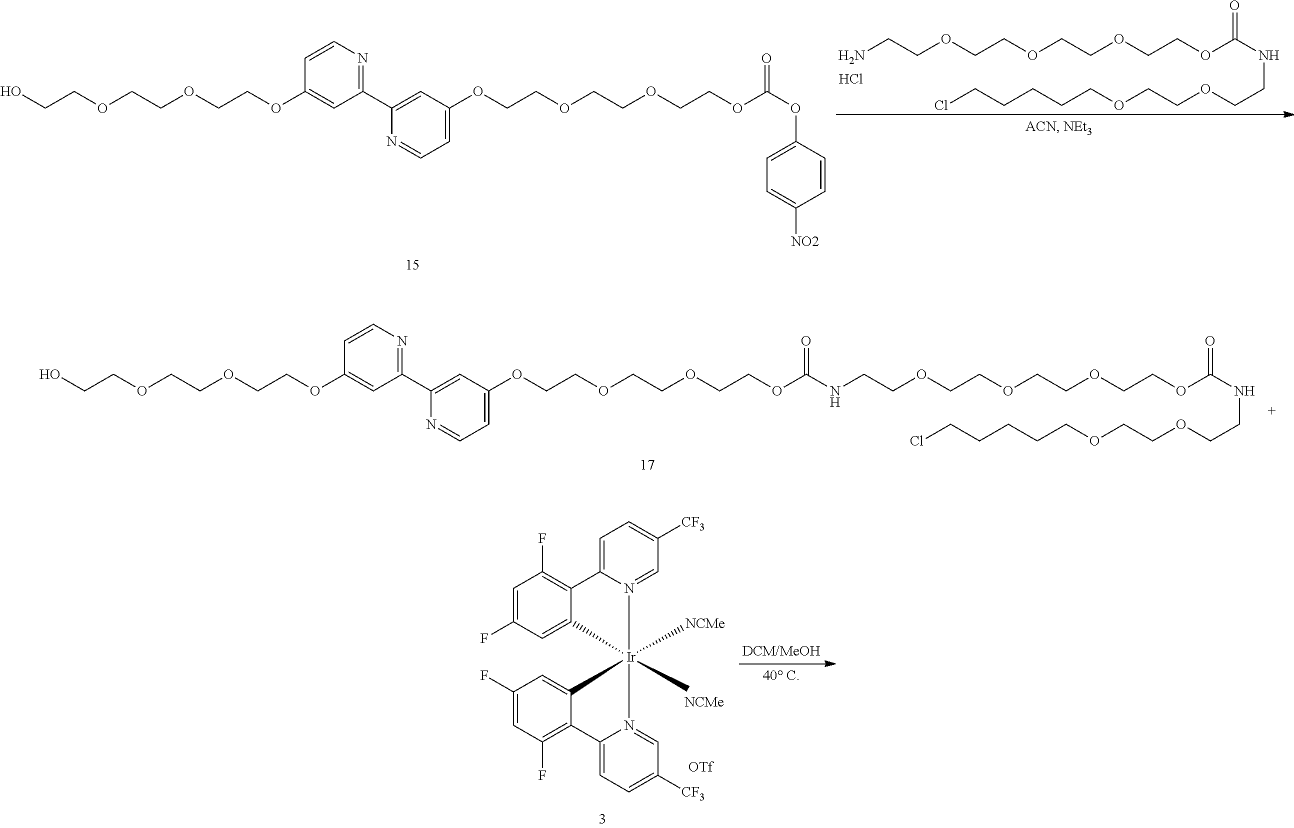

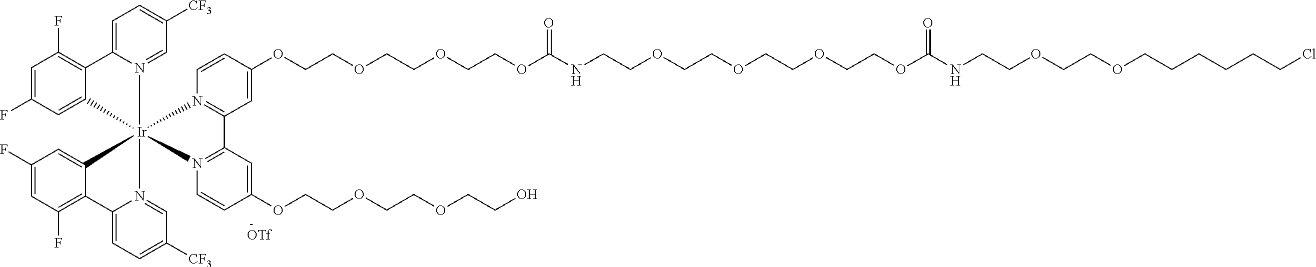

Intermediate 17: To a solution of the crude 15 (30 mg, 49 μmol, 1.0 equiv) in ACN (2 mL), the chloroalkane amine reactive agent (23 mg, 49 μmol, 1.0 equiv) and NEt₃ (0.2 mL) was added. The solution was stirred at RT overnight and concentrated onto celite. The desired product was isolated using silica gel chromatography. ¹H NMR (400 MHZ, Methanol-d₄) δ8.47 (d, J=5.8 Hz, 2H), 7.88 (t, J=2.7 Hz, 2H), 7.06 (d, J=5.7 Hz, 2H), 4.34 (d, J=4.8 Hz, 4H), 4.16 (d, J=4.9 Hz, 4H), 3.93 (h, J=2.9 Hz, 4H), 3.82-3.44 (m, 38H), 3.33-3.25 (m, 2H), 1.77 (t, J=7.3 Hz, 2H), 1.60 (t, J=7.1 Hz, 2H), 1.44 (dq, J=23.7, 7.7 Hz, 4H). LRMS [M+H]⁺921.4.

Ir-8973: Intermediate 3 (4.3 mg, 4.6 umol, 1.0 equiv) and intermediate 17 (4.2 mg, 4.6 μmol, 1.0 equiv) was dissolved in DCM/MeOH (1/1, 2 mL). The solution was stirred at RT overnight. The desired product was isolated by silica column with DCM/MeOH as eluent. ¹H NMR (400 MHz, Methanol-d₄) δ8.56 (d, J=8.9 Hz, 2H), 8.42-8.25 (m, 4H), 7.82 (d, J=4.9 Hz, 4H), 7.25 (d, J=6.4 Hz, 2H), 6.97-6.68 (m, 4H), 5.85-5.68 (m, 2H), 4.49-4.41 (m, 4H), 4.15-4.04 (m, 4H), 3.94-3.85 (m, 4H), 3.73-3.41 (m, 38H), 3.30-3.24 (m, 2H), 1.81-1.68 (m, 2H), 1.63-1.50 (m, 2H), 1.50-1.26 (m, 4H). LRMS [M+H]⁺1410.4.

Synthesis of Ir-9049

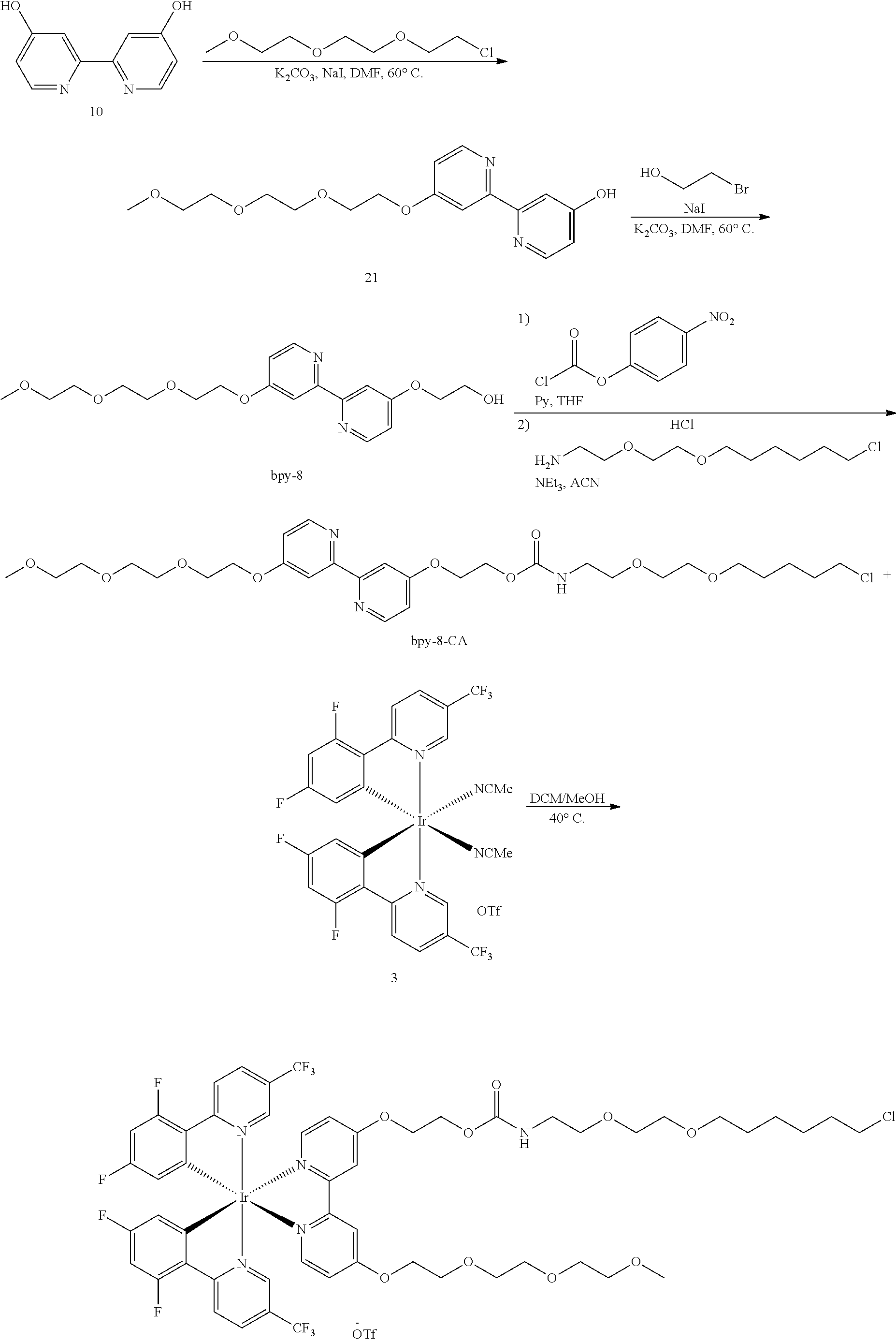

Intermediate 21: To a solution of 10 (94 mg, 0.5 mmol, 1.0 equiv) in DMF (5 mL), NaI (7.5 mg, 0.05 mmol, 0.1 equiv), 1-chloro-2-(2-(2-methoxyethoxy) etho-xy) ethane (110 mg, 0.6 mmol, 1.2 equiv), and K₂CO₃ (207 mg, 1.5 mmol, 3.0 equiv) was added. The suspension was heated at 60° C. overnight. After cooling down, the mixture was diluted with EtOAc (50 mL), filtered over Celite, and the filtrate was concentrated in vacuo to afford the crude. The desired product was isolated by silica gel chromatography. ¹H NMR (400 MHZ, Methanol-d₄) δ8.63 (d, J=5.9 Hz, 1H), 8.46 (dd, J=6.7, 2.1 Hz, 1H), 7.90 (s, 1H), 7.85 (s, 1H), 7.32-7.23 (m, 1H), 7.20 (d, J=6.7 Hz, 1H), 4.43 (t, J=4.6 Hz, 2H), 3.93 (p, J=2.2 Hz, 2H), 3.76-3.47 (m, 8H), 3.35 (s, 3H). LRMS [M+H]⁺335.4.

bpy-8: To a solution of 21 (25 mg, 75 μmol, 1.0 equiv) in DMF (5 mL), bromoethanol (47 mg, 374 μmol, 5.0 equiv), NaI (1.2 mg, 7.5 μmol, 0.1 equiv), and K₂CO₃ (31 mg, 224 μmol, 3.0 equiv) was added. The mixture was stirred at 60° C. overnight. After cooling down, the mixture was diluted with EtOAc (50 mL), filtered over Celite, and the filtrate was concentrated in vacuo to afford the crude. The desired product was isolated by silica gel chromatography. ¹H NMR (400 MHZ, Chloroform-d) δ8.66-8.44 (m, 2H), 8.13-7.75 (m, 2H), 7.00-6.85 (m, 2H), 6.79 (brs, 1H), 4.37-4.20 (m, 4H), 4.04-3.58 (m, 12H), 3.34 (s, 3H). LRMS [M+H]⁺379.4.

bpy-8-CA: To a solution of bpy-8 (16 mg, 0.04 mmol, 1.0 equiv) in THF (4 mL), pyridine (0.5 mL) and p-nitrophenyl chloroformate (10 mg, 0.05 mmol, 1.2 equiv) was added. The solution was stirred at RT overnight. The reaction was diluted with DCM (10 mL), filtered over Celite, and the filtrate was concentrated in vacuo to afford the crude, which was used in the next step without further purification.

To a solution of the crude from previous step in ACN (2 mL), the chloroalkane amine reactive agent (39 mg, 150 μmol, 3 equiv) and NEt₃ (0.2 mL) was added. The solution was stirred at RT overnight and concentrated onto celite. The desired product was isolated using silica gel chromatography. ¹H NMR (400 MHZ, Methanol-d₄) δ8.48-8.46 (m, 2H), 7.95-7.74 (m, 2H), 7.17-6.92 (m, 2H), 4.47-4.25 (m, 6H), 3.95-3.42 (m, 20H), 3.35 (s, 3H), 3.29-3.17 (m, 2H), 1.77-1.50 (m, 2H), 1.63-1.50 (m, 2H), 1.47-1.21 (m, 4H). LRMS [M+H]⁺628.3.

Ir-9049: Intermediate 3 (14 mg, 15 μmol, 1.0 equiv) and bpy-8-CA (9.4 mg, 15 μmol, 1.0 equiv) was dissolved in DCM/MeOH (1/1, 2 mL). The solution was stirred at RT overnight. The desired product was isolated by silica column with DCM/MeOH as eluent. ¹H NMR (400 MHZ, Methanol-d₄) δ8.58 (d, J=8.9 Hz, 2H), 8.39 (s, 2H), 8.33 (d, J=8.9 Hz, 2H), 7.84 (d, J=6.9 Hz, 4H), 7.27 (s, 2H), 6.81 (t, J=10.9 Hz, 2H), 5.79 (d, J=8.0 Hz, 2H), 4.47-4.25 (m, 4H), 3.92-3.78 (m, 2H), 3.75-3.58 (m, 20H), 3.32 (s, 3H), 1.78-1.70 (m, 2H), 1.63-1.50 (m, 2H), 1.47-1.37 (m, 4H). LRMS [M+H]⁺1336.7.

Synthesis of Ir-9050

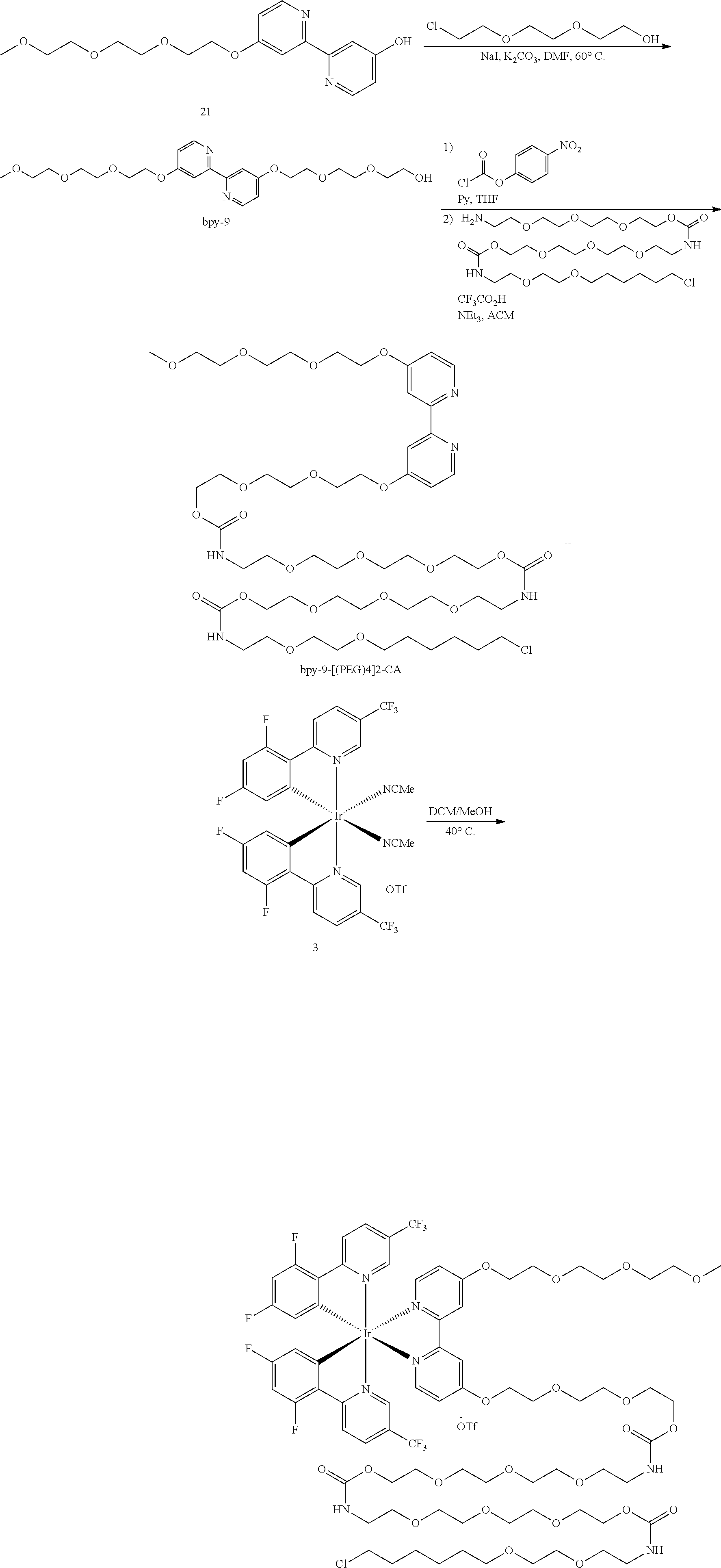

bpy-9: To a solution of 21 (25 mg, 75 μmol, 1.0 equiv) in DMF (5 mL), 2-(2-(2-chloroethoxy) ethoxy) ethan-1-ol (63 mg, 374 μmol, 5.0 equiv), NaI (1.2 mg, 7.5 μmol, 0.1 equiv), and K₂CO₃ (31 mg, 224 μmol, 3.0 equiv) was added. The mixture was stirred at 60° C. overnight. After cooling down, the mixture was diluted with EtOAc (50 mL), filtered over Celite, and the filtrate was concentrated in vacuo to afford the crude. The desired product was isolated by silica gel chromatography. ¹H NMR (400 MHZ, Methanol-d₄) δ8.60-8.35 (m, 2H), 7.99-7.55 (m, 2H), 7.15-6.94 (m, 2H), 4.38-4.27 (m, 4H), 3.90 (d, J=4.7 Hz, 4H), 3.76-3.47 (m, 16H), 3.30 (s, 3H). LRMS [M+H]⁺467.5.

bpy-9-[(PEG) 4]2-CA: To a solution of bpy-9 (16 mg, 0.35 mmol, 1.0 equiv) in THF (4 mL), pyridine (0.5 mL) and p-nitrophenyl chloroformate (8.3 mg, 0.04 mmol, 1.2 equiv) was added. The solution was stirred at RT overnight. The reaction was diluted with DCM (10 mL), filtered over Celite, and the filtrate was concentrated in vacuo to afford the crude, which was used in the next step without further purification.

To a solution of the crude from previous step in ACN (2 mL), the chloroalkane amine reactive agent (54 mg, 150 μmol, 3 equiv) and NEt₃ (0.2 mL) was added. The solution was stirred at RT overnight and concentrated onto celite. The desired product was isolated using silica gel chromatography. LRMS [M+H]⁺1154.6.

Ir-9050: Intermediate 3 (14 mg, 15 umol, 1.0 equiv) and bpy-9-CA (9.4 mg, 15 μmol, 1.0 equiv) was dissolved in DCM/MeOH (1/1, 2 mL). The solution was stirred at RT overnight. The desired product was isolated by silica column with DCM/MeOH as eluent. ¹H NMR (400 MHZ, Methanol-d₄) δ8.58 (d, J=8.9 Hz, 2H), 8.39 (s, 2H), 8.33 (d, J=8.9 Hz, 2H), 7.84 (d, J=6.9 Hz, 4H), 7.27 (s, 2H), 6.81 (t, J=10.9 H

### Syntheses of Ru Catalysts

Ru-8975: The desired product was isolated as di-acetate salt. ¹H NMR (400 MHZ, Methanol-d₄) δ8.90 (d, J=8.5 Hz, 1H), 8.83-8.70 (m, 4H), 8.31 (d, J=8.5 Hz, 1H), 8.25-8.03 (m, 5H), 7.94 (dd, J=12.4, 5.6 Hz, 2H), 7.78 (s, 1H), 7.75-7.62 (m, 3H), 7.60-7.48 (m, 3H), 7.36 (d, J=6.4 Hz, 2H), 7.03 (s, 1H), 3.81 (t, J=6.0 Hz, 2H), 3.59 (t, J=7.1 Hz, 2H), 2.07 (t, J=6.6 Hz, 2H), 1.90 (s, 6H). LRMS [M]²⁺/2 333.6.

### Synthesis of phenanthroline-1

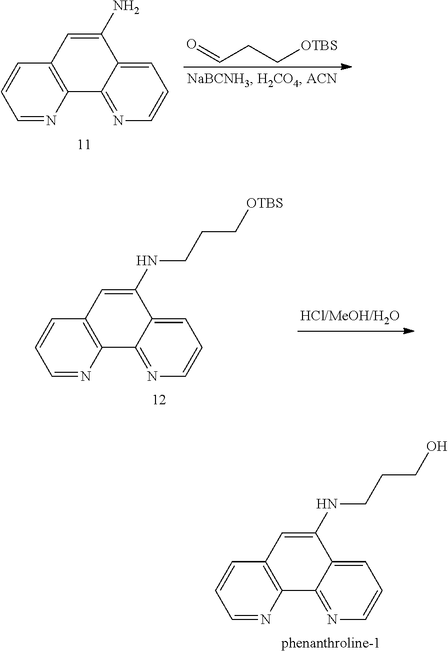

Phenanthroline-1: Phenanthroline 11 (195 mg, 1.0 mmol, 1.0 equiv), 3-((tert-butyldimethylsilyl)oxy)propanal (188 mg, 1.0 mmol, 1.0 equiv) was dissolved in ACN (10 mL) and conc. H₂SO₄ (0.1 mL) was added. The solution was stirred at RT for 30 min before NaCNBH₃ (94 mg, 1.5 mmol, 1.5 equiv) was added in one portion. The reaction mixture was then stirred at RT for additional 3 h before quenched by addition of sat. aqueous NaHCO₃ solution (1 mL). The mixture was then diluted with H₂O (30 mL) and extracted with EtOAc (30×3 mL). The combined organic layers were washed with H₂O (50 mL) and brine (50 mL), dried over Na₂SO₄, and concentrated in vacuo. The desired product 12 was purified by silica gel chromatography. ¹H NMR (400 MHZ, Methanol-d₄) δ9.18-8.92 (m, 1H), 8.80-8.51 (m, 2H), 8.12 (d, J=8.1 Hz, 1H), 7.81-7.45 (m, 2H), 6.75 (s, 1H), 3.89 (t, J=6.0 Hz, 2H), 3.48 (t, J =6.9 Hz, 2H), 2.18-1.88 (m, 2H), 0.92 (s, 9H), 0.09 (s, 6H). LRMS [M+H]⁺368.5.

Phenanthroline intermediate 12 (120 mg, 0.33 mmol, 1.0 equiv) was dissolved in MeOH/6N aq. HCl (1/1, 6 mL). The solution was stirred at RT for 6 h. LC-MS indicated full conversion. The desired product, phenanthroline-1, was isolated by silica gel chromatography. ¹H NMR (400 MHz, Methanol-d₄) δ9.07 (d, J=4.2 Hz, 1H), 8.68 (dd, J=20.8, 6.3 Hz, 2H), 8.15 (d, J=8.1 Hz, 1H), 7.74 (dd, J=9.0, 4.1 Hz, 1H), 7.61-7.46 (m, 1H), 6.80 (s, 1H), 3.91-3.80 (m, 2H), 3.52 (t, J=7.1 Hz, 2H), 2.14-1.99 (m, 2H). LRMS [M+H]⁺254.3.

Synthesis of Ru-8974

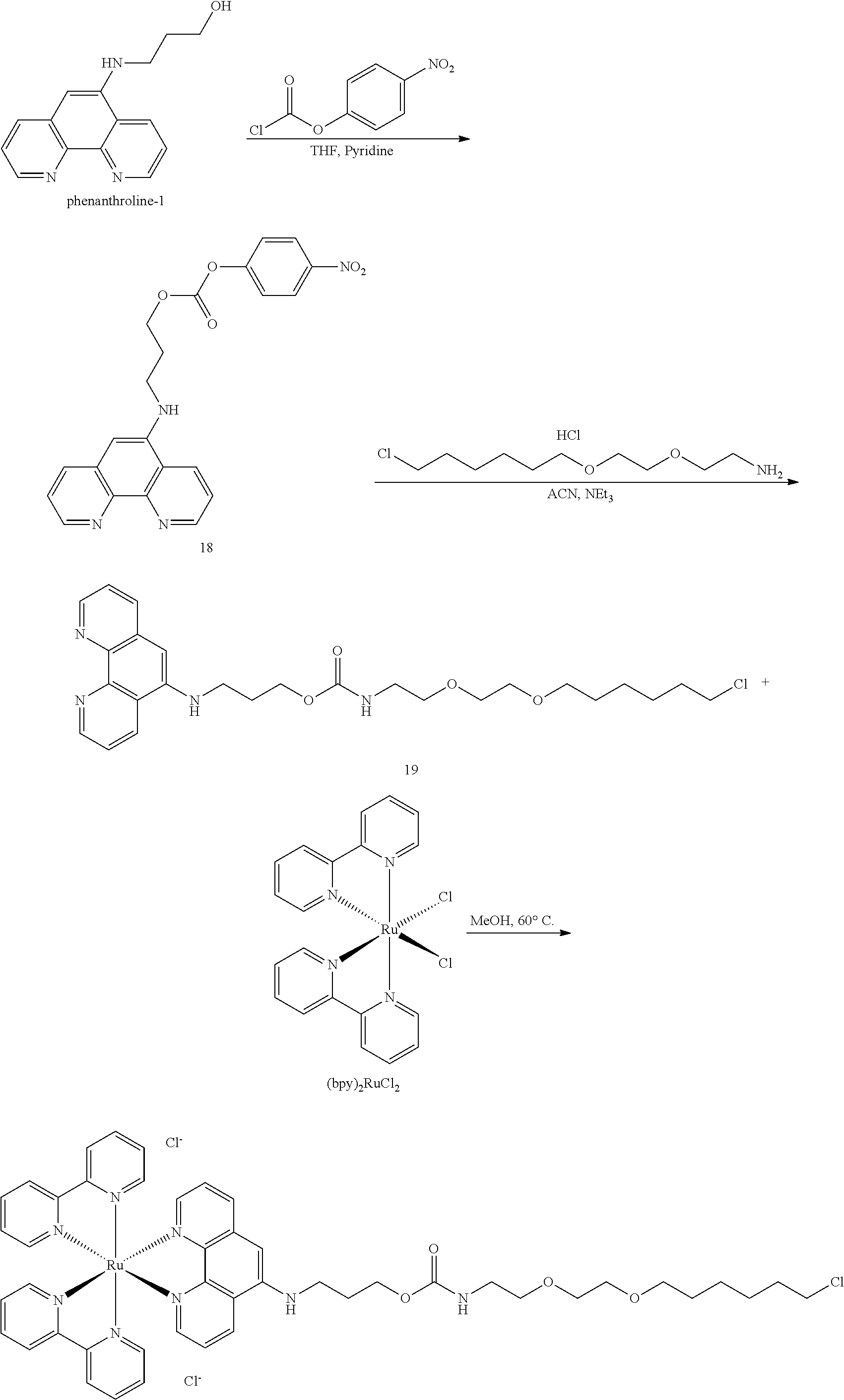

Intermediate 19: To a solution of phenanthroline-1 (16 mg, 0.06 mmol, 1.0 equiv) in THF (4 mL), pyridine (0.5 mL) and p-nitrophenyl chloroformate (13 mg, 0.06 mmol, 1.0 equiv) was added. The solution was stirred at RT overnight. The reaction was diluted with DCM (10 mL), filtered over Celite, and the filtrate was concentrated in vacuo to afford the crude, intermediate 18, which was used in the next step without further purification.

To a solution of the crude 18 (26 mg, 62 μmol, 1.0 equiv) in ACN (2 mL), the chloroalkane amine reactive agent (16 mg, 62 μmol, 1.0 equiv) and NEt₃ (0.2 mL) was added. The solution was stirred at RT overnight and concentrated onto celite. The desired product was isolated using silica gel chromatography. ¹H NMR (400 MHZ, Methanol-d₄) δ9.07 (d, J=4.3 Hz, 1H), 8.71 (t, J=7.6 Hz, 2H), 8.16 (d, J=8.1 Hz, 1H), 7.75 (dd, J=8.8, 4.1 Hz, 1H), 7.68-7.44 (m, 1H), 6.80 (s, 1H), 4.29 (t, J=6.3 Hz, 2H), 3.60-3.25 (m, 14H), 2.25-2.15 (d, J=6.6 Hz, 2H), 1.81-1.68 (m, 2H), 1.63-1.50 (m, 2H), 1.43-1.17 (m, 6H). LRMS [M +H]⁺503.3.

Ru-8974: (bpy)₂RuCl₂ (7.0 mg, 14 μmol, 1.1 equiv) and intermediate 19 (6.6 mg, 13 μmol, 1.0 equiv) was dissolved in MeOH (2 mL). The solution was stirred at 60° C. overnight. The desired product was isolated by silica column with DCM/MeOH as eluent. ¹H NMR (400 MHZ, Methanol-d₄) δ8.92 (d, J=8.6 Hz, 1H), 8.81-8.60 (m, 4H), 8.32 (d, J=8.4 Hz, 1H), 8.12 (dt, J=28.6, 9.3 Hz, 5H), 7.94 (dd, J=12.0, 5.6 Hz, 2H), 7.82-7.62 (m, 4H), 7.55 (p, J=6.6, 5.9 Hz, 3H), 7.36 (d, J=6.6 Hz, 2H), 7.04 (s, 1H), 4.36-4.15 (m, 2H), 3.67-3.41 (m, 14H), 3.30-3.17 (m, 2H), 2.25-2.15 (m, 2H), 1.81-1.68 (m, 2H), 1.63-1.50 (m, 2H), 1.43-1.17 (m, 6H). LRMS [M]²⁺/2 458.1.

Synthesis of Ru-9003

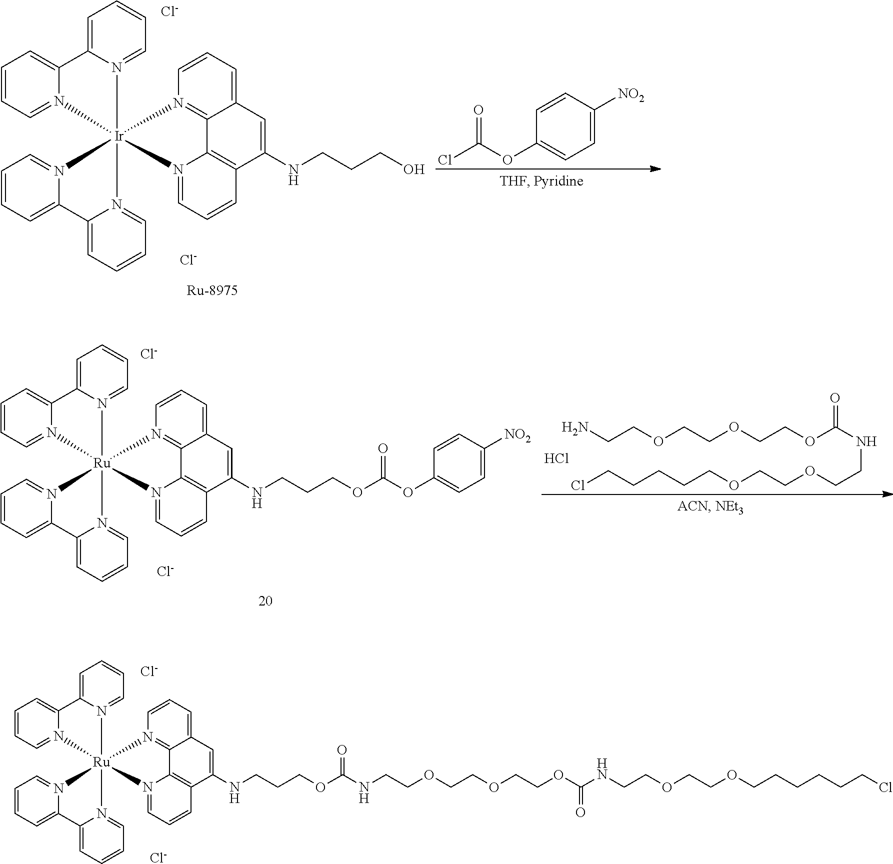

Ru-9003: To a solution of the Ru-8975 (10 mg, 13 μmol, 1.0 equiv) in ACN (2 mL), pyridine (0.5 mL) and p-nitrophenyl chloroformate (7.7 mg, 39 μmol, 3.0 equiv) was added. The solution was stirred at RT overnight. The reaction was diluted with DCM (10 mL), filtered over Celite, and the filtrate was concentrated in vacuo to afford the crude, intermediate 20, which was used in the next step without further purification.

To the solution of crude intermediate 20 was redissolved in ACN (3 mL), the chloroalkane intermediate (32 mg, 39 μmol, 3.0 equiv) and NEt₃ (0.2 mL) was added. The solution was stirred at RT overnight. The desired product was isolated by silica column with DCM/MeOH as eluent. ¹H NMR (400 MHZ, Methanol-d₄) δ8.93 (d, J=8.6 Hz, 1H), 8.81-8.66 (m, 6H), 8.33 (d, J=8.3 Hz, 1H), 8.23-8.05 (m, 7H), 7.95 (dd, J=12.0, 5.6 Hz, 3H), 7.79 (t, J=7.0 Hz, 1H), 7.68 (dd, J=15.9, 9.3 Hz, 4H), 7.63-7.51 (m, 5H), 7.38 (d, J=7.0 Hz, 3H), 7.05 (s, 1H), 4.34-4.22 (m, 2H), 4.19-4.05 (m, 2H), 3.82-3.42 (m, 22H), 3.30-3.17 (m, 2H), 2.25-2.15 (m, 2H), 1.80-1.66 (m, 2H), 1.66-1.52 (m, 2H), 1.45-1.19 (m, 4H). LRMS [M]²⁺/2 545.8. z, 2H), 5.79 (d, J=8.0 Hz, 2H), 4.49-4.41 (m, 4H), 4.15-4.04 (m, 4H), 3.94-3.85 (m, 4H), 3.79-3.47 (m, 57H), 3,24-3.15 (m, 2H), 1.90-1.67 (m, 2H), 1.67-1.47 (m, 2H), 1.46-1.28 (m, 4H). LRMS [M+H]⁺1863.3.

### Syntheses of Activatable Molecules

### Synthesis of PBI-8977 (Azido-quenched coumarin)

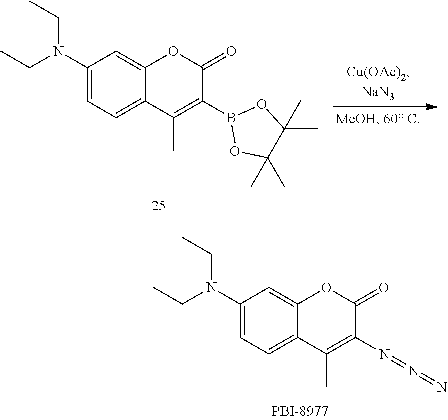

To a solution of intermediate 25 (78 mg, 0.22 mmol, 1.0 equiv) in MeOH (4 mL), Cu (OAc) 2 (4.0 mg, 0.022 mmol, 0.1 equiv) and NaN₃ (14 mg, 0.22 mmol, 1.0 equiv) was added. The reaction was heated at 60° C. for 30 min. LC-MS indicated full conversion. The reaction was diluted with EtOAc (50 mL), then quenched by addition of sat. aq. NH₄Cl (10 mL). The aqueous layer was extracted with EtOAc (10×3 mL). The combined organic layer was washed with H₂O (30 mL) and brine (30 mL), dried over Na₂SO₄, and concentrated in vacuo. The desired product was purified by silica gel chromatography. ¹H NMR (400 MHz, Chloroform-d) δ7.34 (d, J=9.0 Hz, 1H), 6.62 (d, J=8.7 Hz, 1H), 6.50 (d, J=2.7 Hz, 1H), 3.41 (q, J=7.1 Hz, 4H), 2.30 (d, J =1.8 Hz, 3H), 1.20 (t, J=7.1 Hz, 6H). LRMS [M+H]⁺273.13.

### Synthesis of PBI-7273 (p-azidobenzyl-luciferin)

### 6-((4-azidobenzyl) oxy) benzo[d]thiazole-2-carbonitrile

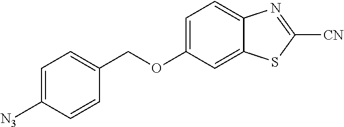 [SMILES: N#Cc1nc2ccc(OCc3ccc(N=[N+]=[N-])cc3)cc2s1]

To a solution of 6-hydroxybenzo[d]thiazole-2-carbonitrile (0.1 g, 0.5 mmol) in acetonitrile (30 ml) was added 1-azido-4-(bromomethyl) benzene (0.12 g, 0.57 mmol), potassium carbonate (0.16 mg, 1.13 mmol) and potassium iodide (0.19 mg, 1.13 mmol). The mixture was refluxed overnight. After cooling down, the solvent was evaporated, and the residue was extracted with ethyl acetate/water. The organic layer was collected, and the solvent was evaporated. The residue was purified by silica gel chromatography to give the product as a yellowish solid, which was used directly in the next step.

### (S)-2-(6- ((4-azidobenzyl) oxy) benzo[d]thiazol-2-yl)-4,5-dihydrothiazole-4-carboxylic acid (PBI-7273)

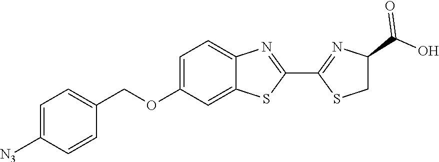 [SMILES: [N-]=[N+]=Nc1ccc(COc2ccc3nc(C4=N[C@@H](C(=O)O)CS4)sc3c2)cc1]

To a solution of 6-((4-azidobenzyl) oxy) benzo[d]0 thiazole-2-carbonitrile (50 mg, 0.153 mmol) in acetonitrile was added a solution of D-cysteine (31 mg, 0.195 mmol) in water. Triethylamine was added to adjust the pH of the reaction to 9. The resulting mixture was stirred for 20 min and directly purified by prep HPLC (0.1% TFA aq and acetonitrile as elute) to give the product of a white powder. MS: calculated: m/z=412.05 [M+H]⁺; measured (ESI): m/z=412.0 [M+H]⁺. 1H NMR (400 MHZ, DMSO-d6) 8:13.20 (s, 1H), 8.07 (d, J=8.0 Hz, 1H), 7.86 (d, J=4.0 Hz, 1H), 7.54 (d, 2H), 7.28 (dd, J=8.0 Hz, 4.0 Hz, 1H), 7.17 (d, 2H), 5.43 (t, 1H), 5.21 (s, 2H), 3.74 (m, 2H).

### Synthesis of PBI-4043 (o-nitrobenzyl-fluoro-luciferin) 5-fluoro-6-((4-nitrobenzyl) oxy) benzo[d]thiazole-2-carbonitrile

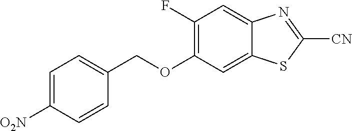 [SMILES: N#Cc1nc2cc(F)c(OCc3ccc([N+](=O)[O-])cc3)cc2s1]

This compound was prepared in a similar method to 6-((4-azidobenzyl) oxy) benzo[d]thiazole-2-carbonitrile and was used directly in the next step.

### (S)-2-(5-fluoro-6- ((4-nitrobenzyl) oxy) benzo[d] thiazol-2-yl)-4,5-dihydrothiazole-4-carboxylic acid (PBI-4043)

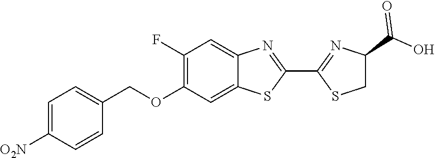 [SMILES: O=C(O)[C@H]1CSC(c2nc3cc(F)c(OCc4ccc([N+](=O)[O-])cc4)cc3s2)=N1]

This compound was prepared in a similar method to PBI-7273. MS: calculated: m/z=472.52 [M+H]⁺; measured (ESI): m/z=472.10 [M+H]⁺. 1H NMR (400 MHZ, DMSO-d6) δ:7.3-8.2 (m, 6H), 5.60 (s, 2H), 4.82 (m, 1H), 3.4-3.8 (m, 2H).

SEQUENCES

SEQ ID NO: 1-NanoLuc-
MKHHHHHHAIAMVFTLEDFVGDWRQTAGYNLDQVLEQGGVSSLFQNLGVSVTPIQRIV
LSGENGLKIDIHVIIPYEGLSGDQMGQIEKIFKVVYPVDDHHFKVILHYGTLVIDGVTPNM
IDYFGRPYEGIAVFDGKKITVTGTLWNGNKIIDERLINPDGSLLFRVTINGVTGWRLCERI
LAV
SEQ ID NO: 2-full-length NanoBIT-
MVFTLEDFVGDWEQTAAYNLDQVLEQGGVSSLLQNLAVSVTPIQRIVRSGENALKIDIH
VIIPYEGLSADQMAQIEEVFKVVYPVDDHHFKVILPYGTLVIDGVTPNMLNYFGRPYEGI
AVFDGKKITVTGTLWNGNKIIDERLITPDGSMLFRVTINSVTGYRLFEEIL
SEQ ID NO: 3-HiBIT-VSGWRLFKKIS
SEQ ID NO: 4-SmBIT-VTGYRLFEEIL
SEQ ID NO: 5-LgBIT-
MVFTLEDFVGDWEQTAAYNLDQVLEQGGVSSLLQNLAVSVTPIQRIVRSGENALKIDIH
VIIPYEGLSADQMAQIEEVFKVVYPVDDHHFKVILPYGTLVIDGVTPNMLNYFGRPYEGI
AVFDGKKITVTGTLWNGNKIIDERLITPDGSMLFRVTINSHHHHHH
SEQ ID NO: 6-LgTrip-
MVFTLEDFVGDWEQTAAYNLDQVLEQGGVSSLLQNLAVSVTPIQRIVRSGENALKIDIH
VIIPYEGLSADQMAQIEEVFKVVYPVDDHHFKVILPYGTLVIDGVTPNMLNYFGRPYEGI
AVFDGKKITVTGTLWNGNKIIDERLITPD
SEQ ID NO: 7-SmTrip9-GSMLFRVTINS
SEQ ID NO: 8-HaloTag-
MAEIGTGFPFDPHYVEVLGERMHYVDVGPRDGTPVLFLHGNPTSSYVWRNIIPHVAPTH
RCIAPDLIGMGKSDKPDLGYFFDDHVRFMDAFIEALGLEEVVLVIHDWGSALGFHWAK
RNPERVKGIAFMEFIRPIPTWDEWPEFARETFQAFRTTDVGRKLIIDQNVFIEGTLPMGVV
RPLTEVEMDHYREPFLNPVDREPLWRFPNELPIAGEPANIVALVEEYMDWLHQSPVPKL
LFWGTPGVLIPPAEAARLAKSLPNCKAVDIGPGLNLLQEDNPDLIGSEIARWLSTLEISG

1. A system comprising:
(a) a bioluminescent protein or structurally-complementary components of a bioluminescent complex;
(b) a luminophore, wherein the bioluminescent protein or complex catalyzes emission of a first wavelength of light from the luminophore upon interaction therewith; and
(c) a photocatalyst, wherein the photocatalyst is activated by exposure to light of the first wavelength;

2. [A] The system of claim 1, further comprising:
(d) an activatable molecule, wherein the activatable molecule is converted into an activated molecule when in proximity to the activated photocatalyst.

3-4. (canceled)

5. The system of claim 1, wherein the bioluminescent protein is a luciferase with 100% sequence identity with SEQ ID NO: 1.

6. The system of claim 1, wherein the luciferase is a circularly permuted variant of an Oplophorous-derived polypeptide.

7. (canceled)

8. The system of claim 1, wherein the first segment comprises 100% sequence identity with the first portion of SEQ ID NO: 1 and the second segment comprises 100% sequence identity with the second portion of SEQ ID NO: 1.

9-11. (canceled)

12. The system of claim 1, wherein the structurally-complementary components collectively comprise at least 70% sequence identity with SEQ ID NO: 2.

13. (canceled).

14. The system of claim 13, wherein the structurally-complementary components comprise (1) a peptide with 100% sequence identity with SEQ ID NO: 3 or 4 and a polypeptide with 100% sequence identity with SEQ ID NO: 5, or (2) peptide with 100% sequence identity with SEQ ID NO: 3 or 4, a polypeptide with 100% sequence identity with SEQ ID NO: 6, and a peptide with 100% sequence identity with SEQ ID NO: 7.

15-16. (canceled)

17. The system of claim 1, wherein the polypeptide component is circularly permuted.

18. The system of claim 1, wherein the luminophore is a luciferin or a coelenterazine molecule.

19. (canceled)

20. The system of claim 18, wherein the coelenterazine molecule is furimazine or fluorofurimazine.

21. The system of claim 1, wherein the photocatalyst is an iridium-based or ruthenium-based photocatalyst.

22. The system of claim 21, wherein the photocatalyst is of the structure of Formula (I): 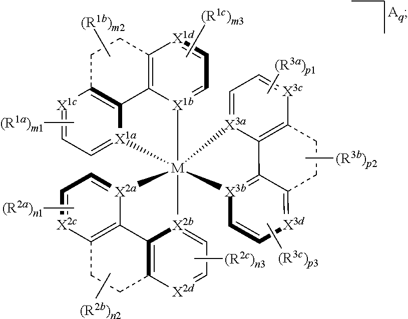
wherein:
each set of dashed lines (------) represents the presence or absence of a fused 6-membered ring;
M is a transition metal;
m1, m2, m3, n1, n2, n3, p1, p2, and p3 are each independently 0, 1, or 2;
R^{1a}, R^{1b}, R^{1c}, R^{2a}, R^{2b}, R^{2c}, R^{3a}, R^{3b}, and R^{3c} are each independently selected from halo, alkyl, haloalkyl, amino, heteroalkyl, and a group-Linker-Q, wherein Q is a capture element;
X^{1a}, X^{1b}, X^{2a}, X^{2b}, X^{3a}, and X^{3b} are each independently selected from N and C, wherein at least one of X^{1a} and X^{1b} is N, at least one of X^{2a} and X^{2b} is N, and at least one of X^{3a} and X^{3b} is N;
X^{1c}, X^{1d}, X^{2c}, X^{2d}, X^{3c}, and X^{3d} are each independently selected from CH and N; A is an anion; and
q is 0, 1, or 2.

23. The system of claim 22, wherein the transition metal is selected from Ru and Ir.

24. The system of claim 23, wherein the photocatalyst is an iridium-based photocatalyst selected from: 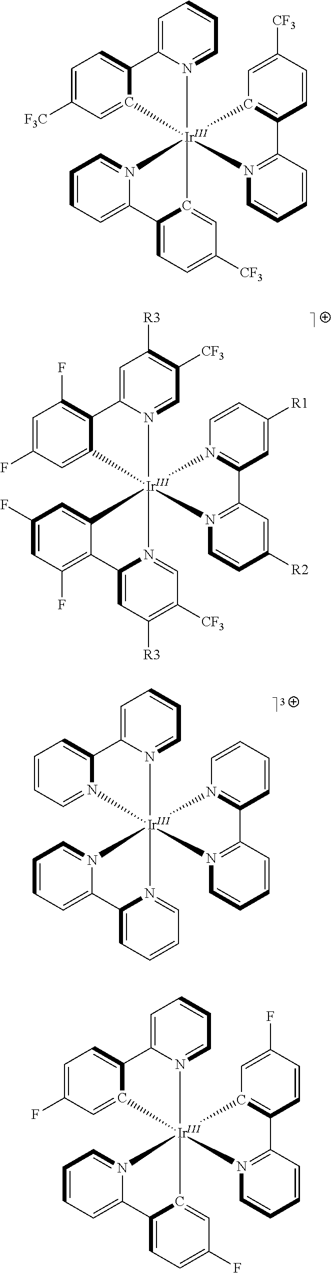 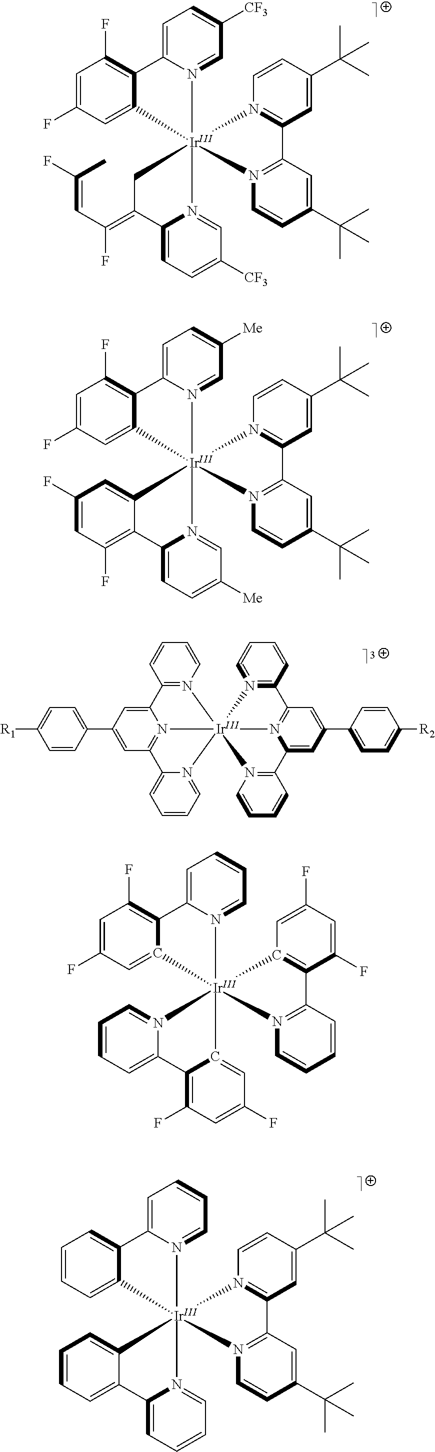 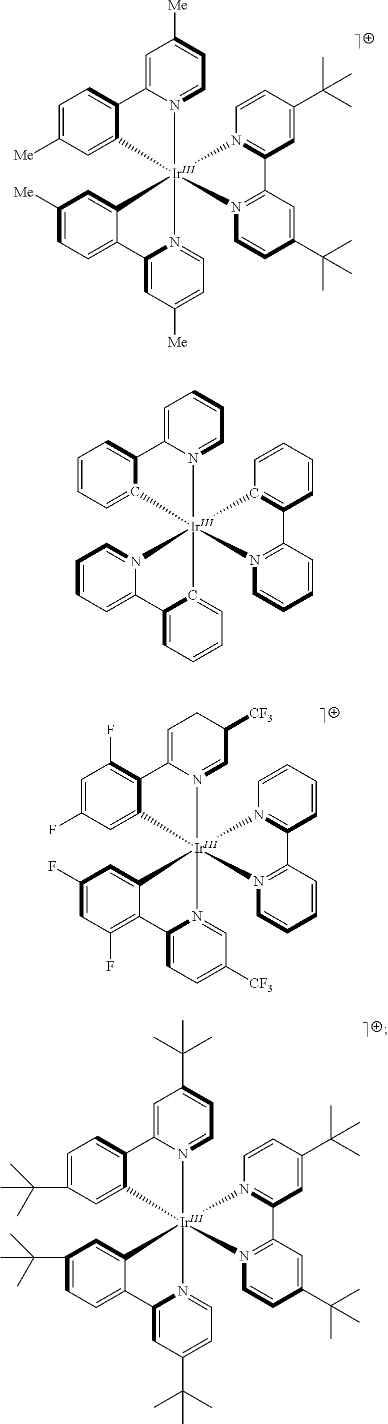
or a derivative thereof in which the compound is functionalized with at least one group-Linker-Q, wherein Q is a capture element.

25. The system of claim 23, wherein the photocatalyst is a ruthenium-based photocatalyst selected from: 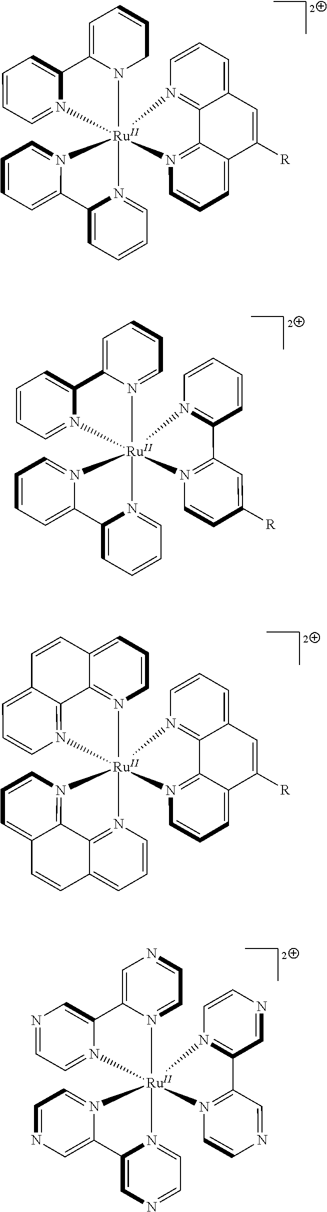 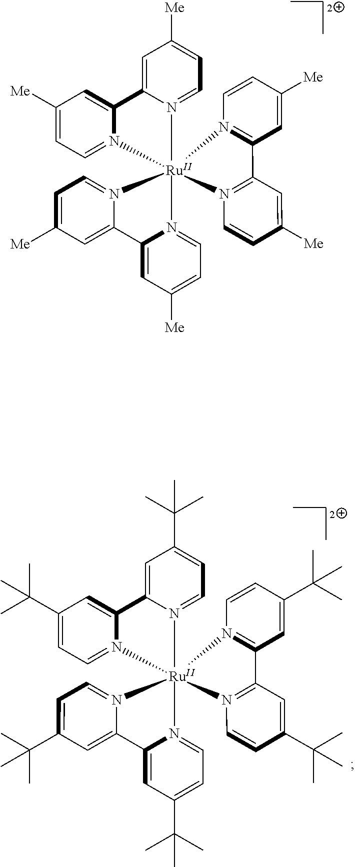
or a derivative thereof in which the compound is functionalized with at least one group-Linker-Q, wherein Q is a capture element.

26. The system of claim 22. wherein the photocatalyst is of the formula:

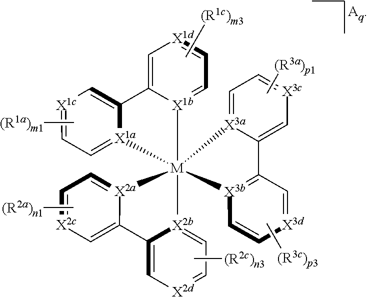

27. The system of claim 22, wherein one of R^{1a}, R^{1b}, R^{1c}, R^{2a}, R^{2b}, R^{2c}, R^{3a}, R^{3b}, and R^{3c} is a group-Linker-Q.

28. The system of claim 27, wherein Q is a capture element.

29. The system of claim 28, wherein Q is a haloalkane.

30. The system of claim 27, wherein the Linker comprises ester (—C(O)O—), amide (—C(O)NH—), carbamate (—NHC(O)O—), urea (—NHC(O)NH—), phenylene (e.g., 1,4-phenylene), straight or branched chain alkylene, oligo-or poly-ethylene glycol (—(CH₂CH₂O)ₓ—), or combinations thereof.

31. The system of claim 30, wherein the Linker comprises —O(CH₂CH₂O)_{z1}—C(O)NH—(CH₂CH₂O)_{z2}—C(O)NH—(CH₂)_{z3}—(OCH₂CH₂)_{z4}O—, wherein z1, z2, z3, and z4 are each independently selected form 0, 1, 2, 3, 4, 5, and 6.

32. The system of claim 31, wherein the Linker is selected from:

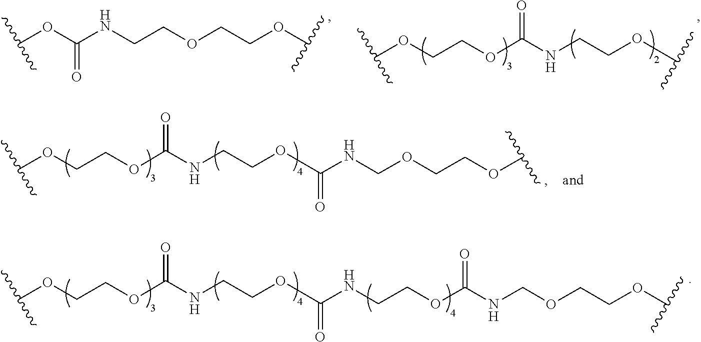

33. The system of claim 1, wherein the photocatalyst is an organic photoredox catalyst.

34. (canceled)

35. The system of claim 33, wherein the organic photoredox catalyst is a quinone selected from:

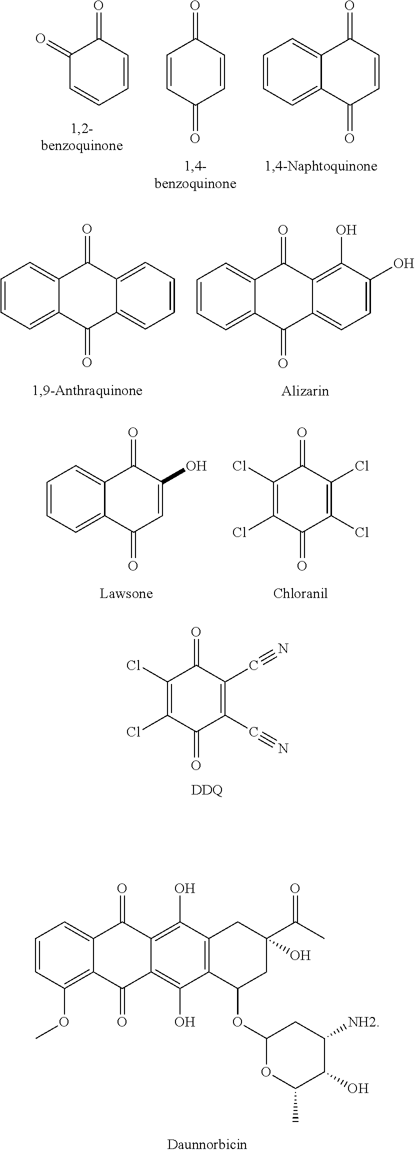

36. The system of claim 33, wherein the organic photoredox catalyst is a pyrylium selected from:

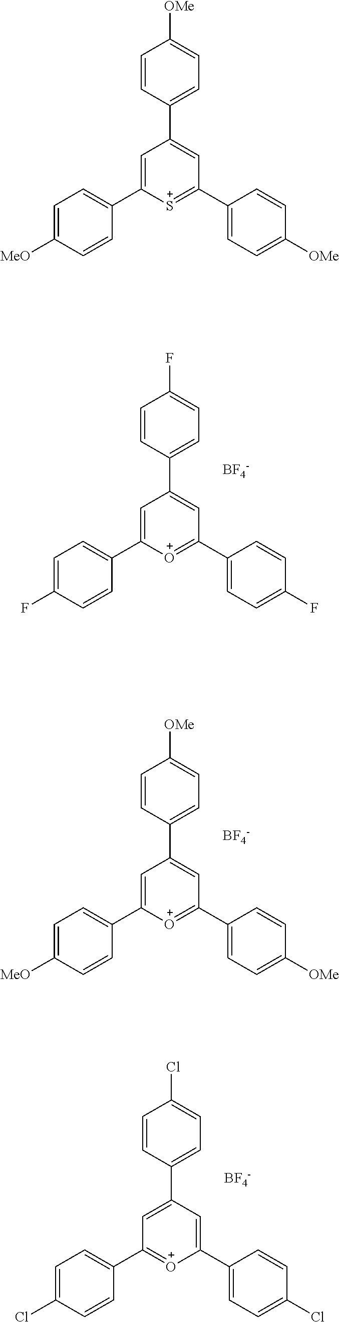

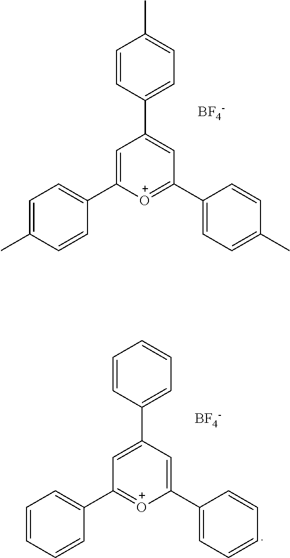

37. The system of claim 33, wherein the organic photoredox catalyst is an acridinium selected from:

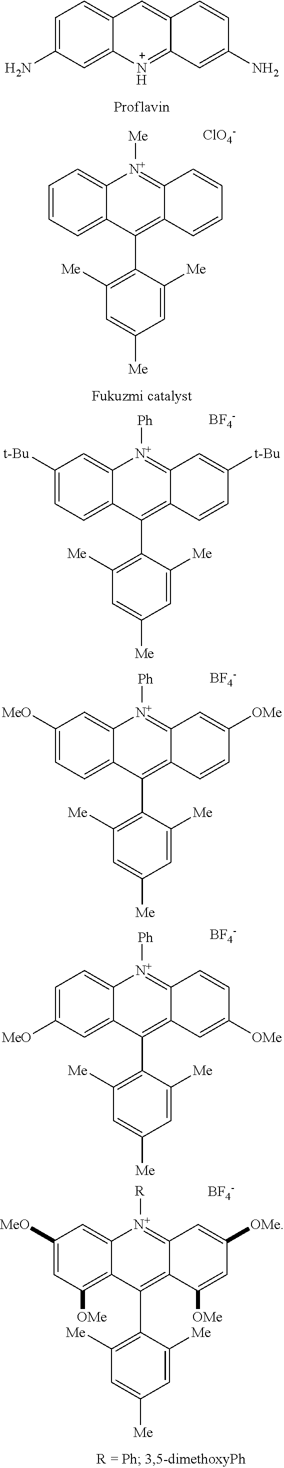

38. The system of claim 33, wherein the organic photoredox catalyst is a xanthene selected from:

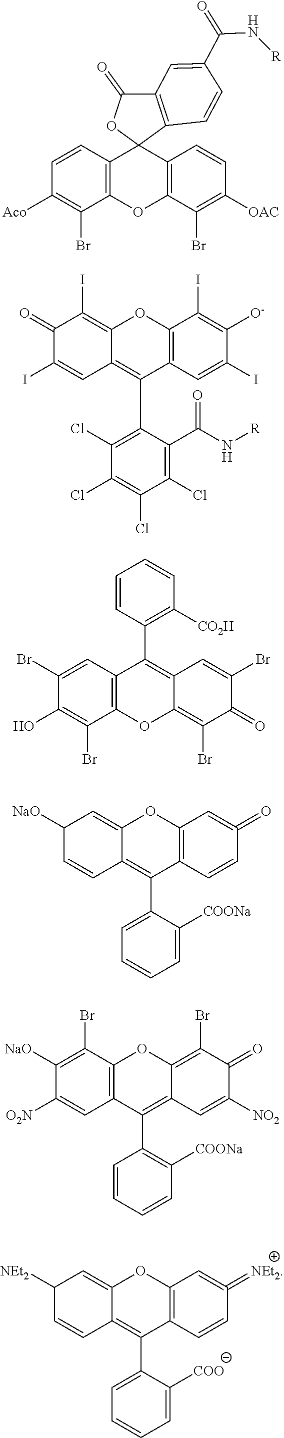

39. The system of claim 33, wherein the organic photoredox catalyst is a thiazine-based organic photoredox catalyst.

40. (canceled)

41. The system of claim 1, wherein the first wavelength of light is between 400 and 500 nm.

42. The system of claim 1, wherein the photocatalyst facilitates (i) energy transfer to the activatable molecule, (ii) abstraction of a hydrogen from the activatable molecule, or (iii) catalysis of a photoredox reaction.

43. The system of claim 1, wherein the photocatalyst transfers energy to the activatable molecule by Förster Resonance Energy Transfer, Dexter Energy Transfer, Single Electron Transfer, Singlet oxygen, or photocatalyst-driven conformational change.

44. The system of claim 1, wherein the activatable molecule is a caged molecule or a photoswitchable molecule.

45. The system of claim 44, wherein the activatable molecule is a caged molecule comprising a functional moiety and a blocking moiety.

46. The system of claim 45, wherein the photocatalyst facilitates cleavage of the blocking moiety from the functional moiety, thereby activating the activatable molecule.

47. The system of claim 44, wherein the blocking moiety is selected from:

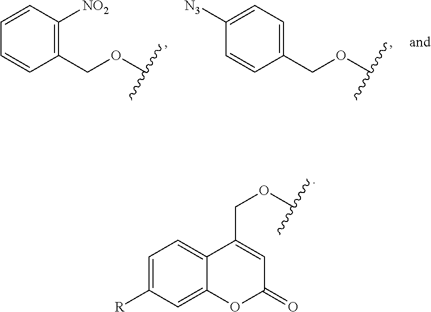

48. The system of claim 44, wherein the blocking moiety prevents binding of the functional moiety to a target and/or detection.

49. (canceled)

50. The system of claim 44, wherein the activatable molecule is a photoswitchable molecule is an inactive conformation, and wherein the photocatalyst facilitates conversion of the photoswitchable molecule from an inactive conformation to an active conformation, thereby activating the activatable molecule.

51. (canceled)

52. The system of claim 50, wherein the photoswitchable molecule comprises first and second functional moieties linked to a photoswitch moiety, wherein when the photoswitchable molecule is in the inactive conformation the first and second functional moieties are not in proximity and/or in a proper orientation to interact, and wherein when the photoswitchable molecule is in the active conformation the first and second functional moieties are in proximity and/or in a proper orientation to interact.

53. The system of claim 52, wherein the photoswitch moiety comprises: 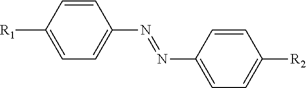
in an inactive conformation and 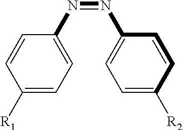
in an active conformation; wherein R₁ and R₂ are the first and second functional moieties.

54. The system of claim 52, wherein the first and second functional moieties are small molecule moieties.

55. The system of claim 1, wherein the bioluminescent protein or component of the bioluminescent complex is fused to a first molecular entity and the photocatalyst or photosensitizer is tethered to a second molecular entity, wherein interaction of the first and second molecular entities places the bioluminescent protein or bioluminescent complex in sufficient proximity to the photocatalyst or photosensitizer such that light emitted by the luminophore upon interaction with the bioluminescent protein or bioluminescent complex activates the photocatalyst or photosensitizer.

56. The system of claim 55, wherein the first molecular entity is a capture agent, and the second molecular entity is a capture element.

57. The system of claim 56, wherein the bioluminescent protein or component of a bioluminescent complex is fused to the N-terminus, the C-terminus, or at an internal site within the capture agent.

58. The system of claim 55, wherein the bioluminescent protein or component of the bioluminescent complex is fused to or inserted into a modified dehalogenase capable of forming a covalent bond with its substrate, and wherein the photocatalyst or photosensitizer is tethered to a dehalogenase substrate.

59. The system of claim 58, wherein binding of the modified dehalogenase to the dehalogenase substrate places the bioluminescent protein or bioluminescent complex in sufficient proximity to the photocatalyst or photosensitizer such that light emitted by the luminophore upon interaction with the bioluminescent protein or bioluminescent complex activates the photocatalyst or photosensitizer.

60. (canceled)

61. The system of claim 1, wherein the modified dehalogenase comprises 100% sequence identity with SEQ ID NO: 8.

62. (canceled)

63. The system of claim 58, wherein the structure of the photocatalyst tethered to the dehalogenase substrate is P-linker-AX, wherein P is the photocatalyst, wherein A is (CH₂)₂₋₁₂, wherein X is a halogen, and wherein the linker is a linker moiety capable of tethering P to A-X.

64. The system of claim 63, wherein the linker is a multiatom straight or branched chain including C, N, S, or O, or a group that comprises one or more aryl rings, heteroaryl rings, or any combination thereof.

65. The system of claim 64, wherein the linker comprises a combination of —O(CH₂)₂— —(CH2)O—, —CH₂—, —NHC(O)O—, —OC(O)NH—, NHC(O)—, and —C(O)NH—.

66. The system of claim 64, wherein the linker is 5 to 50 atoms in length.

67. A cell comprising the system of claim 1.

68. A method of proximity-dependent activation of a molecule within a cell, comprising contacting a cell with a luminophore under conditions in which the luminophore enters the cell, wherein the cell comprises:
(a) a fusion of a bioluminescent protein and a capture protein, wherein the bioluminescent protein catalyzes emission of a first wavelength of light from the luminophore upon interaction therewith;
(b) a conjugate of (A) a capture ligand and (B) a photocatalyst or photosensitizer, wherein the capture protein forms a covalent bond with the capture ligand upon interaction therewith, and wherein the photocatalyst or photosensitizer is activated by exposure to light of the first wavelength; and
(c) an activatable molecule, wherein the activatable molecule is converted into an activated molecule when in proximity to the activated photocatalyst or photosensitizer.

69. A method of proximity-dependent activation of an activatable molecule within a cell, comprising:
(a) expressing a fusion of a bioluminescent protein and a capture protein within the cell;
(b) contacting the cell with a luminophore, under conditions in which the luminophore enters the cell, wherein the bioluminescent protein catalyzes emission of a first wavelength of light from the luminophore upon interaction therewith;
(c) contacting the cell with a conjugate of (i) a capture ligand and (ii) a photocatalyst or photosensitizer, under conditions in which the conjugate enters the cell, wherein the capture protein forms a covalent bond with the capture ligand upon interaction therewith, and wherein the photocatalyst or photosensitizer is activated by exposure to light of the first wavelength; and
(d) contacting the cell with an activatable molecule, wherein the activatable molecule is converted into an activated molecule when in proximity to the activated photocatalyst or photosensitizer.

70. A method of proximity-dependent activation of a photocatalyst or photosensitizer within a cell, comprising contacting a cell with a luminophore under conditions in which the luminophore enters the cell, wherein the cell comprises:
(a) a fusion of a bioluminescent protein and a capture protein, wherein the bioluminescent protein catalyzes emission of a first wavelength of light from the luminophore upon interaction therewith;
(b) a conjugate of (A) a capture ligand and (B) a photocatalyst or photosensitizer, wherein the capture protein forms a covalent bond with the capture ligand upon interaction therewith, and wherein the photocatalyst or photosensitizer is activated by exposure to light of the first wavelength.

71. A method of proximity-dependent activation of a photocatalyst or photosensitizer within a cell, comprising:
(a) expressing a fusion of a bioluminescent protein and a capture protein within the cell;
(b) contacting the cell with a luminophore, under conditions in which the luminophore enters the cell, wherein the bioluminescent protein catalyzes emission of a first wavelength of light from the luminophore upon interaction therewith; and
(c) contacting the cell with a conjugate of (i) a capture ligand and (ii) a photocatalyst or photosensitizer, under conditions in which the conjugate enters the cell, wherein the capture protein forms a covalent bond with the capture ligand upon interaction therewith, and wherein the photocatalyst or photosensitizer is activated by exposure to light of the first wavelength.


SEQUENCE LISTING (8 sequences)

SEQ ID NO: 1 - protein, 183 aa, Synthetic construct
MKHHHHHHAIAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAV

SEQ ID NO: 2 - protein, 170 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSVTGYRLFEEIL

SEQ ID NO: 3 - protein, 11 aa, Synthetic construct
VSGWRLFKKIS

SEQ ID NO: 4 - protein, 11 aa, Synthetic construct
VTGYRLFEEIL

SEQ ID NO: 5 - protein, 165 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 6 - protein, 148 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPD

SEQ ID NO: 7 - protein, 11 aa, Synthetic construct
GSMLFRVTINS

SEQ ID NO: 8 - protein, 297 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG